(19) United States
(12) Patent Application Publication
(10) Pub. No.: US 2013/0065257 A1
(43) Pub. Date: Mar. 14, 2013

(54) ENZYME-LOGIC BIOSENSING

(71) Applicants: Joseph Wang, San Diego, CA (US); Joshua Ray Windmiller, Del Mar, CA (US); Padmanabhan Santhosh, San Diego, CA (US); Min-Chieh Chuang, San Diego, CA (US); Evgeny Katz, Potsdam, NY (US); Jan Halámek, Potsdam, NY (US); Vera Bocharova, Potsdam, NY (US); Marcos Pita, Madrid (ES); Vladimir Privman, Potsdam, NY (US)

(72) Inventors: Joseph Wang, San Diego, CA (US); Joshua Ray Windmiller, Del Mar, CA (US); Padmanabhan Santhosh, San Diego, CA (US); Min-Chieh Chuang, San Diego, CA (US); Evgeny Katz, Potsdam, NY (US); Jan Halámek, Potsdam, NY (US); Vera Bocharova, Potsdam, NY (US); Marcos Pita, Madrid (ES); Vladimir Privman, Potsdam, NY (US)

(21) Appl. No.: 13/635,396

(22) PCT Filed: Mar. 16, 2011
(86) PCT No.: PCT/US2011/028747

§ 371 Date: Nov. 28, 2012

Related U.S. Application Data
(60) Provisional application No. 61/314,494, filed on Mar. 16, 2010.
(60) Provisional application No. 61/329,512, filed on Apr. 29, 2010.

(51) Int. Cl. C12M 1/40; C12Q 1/32; C12Q 1/54; G01N 33/53

(52) U.S. Cl. 435/7.92; 435/287.1; 435/288.7; 435/287.2; 435/26; 435/14

(57) ABSTRACT

Techniques, apparatus and systems are disclosed for implementing enzyme-logic based diagnosis that uses patterns of multiple markers and biochemical processing of the signal information for reliably identifying cardiac abnormalities and providing a final digital binary answer. In one aspect, a biochemical logic sensing system includes a network of enzyme-biocatalyzed logic gates adapted to receive biomarker input signals and perform an enzyme-biocatalyzed reaction resembling a Boolean logic operation using the received biomarker input signals to generate an output signal of the enzyme-biocatalyzed reaction. A signal processing unit is connected to the network of enzyme-biocatalyzed logic gates. The signal processing unit processes the generated output signal of the enzyme-biocatalyzed reaction and generates a digital binary output having a value of zero or one. The generated digital binary output indicates a type of an injury.

### CROSS REFERENCE TO RELATED APPLICATIONS

This international patent application claims the benefit of U.S. Provisional Patent Application No. 61/314,494, filed Mar. 16, 2010, and U.S. Provisional Patent Application No. 61/329,512, filed Apr. 29, 2010. The entire contents of the before-mentioned patent applications are incorporated by reference as part of the disclosure of this application.

### BACKGROUND

This application relates to devices and techniques that use biosensors to perform various medical tests.

Various types of diagnostic tools are available to detect different types of injuries or medical conditions, such as soft tissue injuries (STI), cardiovascular events, etc. For example, sophisticated diagnostic equipment for detection of different medical conditions can include magnetic resonance imaging and electromyography. Also, immunoassays or non-specific enzyme based assays can be used. Recently, chemical computing or biocomputing techniques have been described. Chemical computing is a subarea of unconventional computing that describes processing of information by chemical means. Biocomputing is based on the application of biomolecular systems for processing chemical information.

### SUMMARY

Techniques and structures and apparatus are disclosed for implementing logic based biosensors.

In one aspect, a biochemical logic sensing system includes a network of enzyme-biocatalyzed logic gates adapted to receive biomarker inputs and perform an enzyme-biocatalyzed reaction resembling a Boolean logic operation using the received biomarker inputs to generate an output of the enzyme-biocatalyzed reaction. A signal processing is unit connected to the network of enzyme-biocatalyzed logic gates. The signal processing unit processes the generated output of the enzyme-biocatalyzed reaction and generates a digital binary output having a value of zero or one. The generated digital binary output indicates an injury.

Implementations can optionally include one or more of the following features. The biochemical logic sensing system can include a drug delivery device connected to the signal processing unit. The drug delivery device can identify a dosage of a drug to deliver based on the processed digital binary output. The signal processing unit can be adapted to generate the digital binary output having a value of 0 when the network of enzyme-biocatalyzed logic gates detects a physiologically normal concentration of the biomarker inputs. The signal processing unit can be adapted to generate the digital binary output having a value of 1 when the network of enzyme-biocatalyzed logic gates detects an abnormal concentration of the biomarker input. The biomarker inputs can include glucose, lactate, norepinephrine (NE) and oxygen. The signal processing unit can be adapted to generate the digital binary output having a value of 1 when the network of enzyme-biocatalyzed logic gates detects an abnormal increase in glucose concentration associated with hemorrhagic shock (HS), a higher than normal physiological concentration of lactate associated with HS and/or trauma brain injury (TBI), a high concentration of NE associated with a traumatic injury, a lack of oxygen associated with ischemic state and/or heart attack, or a combination of these.

Implementations can optionally include one or more of the following features. The network of enzyme-biocatalyzed logic gates can include an AND gate, an XOR gate, an OR gate, a NAND gate, a NOR gate, an IDENTITY gate, or a combination of any of these logic gates. The network of enzyme-biocatalyzed logic gates can be adapted to form a cascade arrangement. The network of enzyme-biocatalyzed logic gates can be adapted to form parallel-operating enzyme-catalyzed pathways producing outputs of two or more enzyme-biocatalyzed reactions in parallel. The signal processing unit can be adapted to generate two or more digital binary output in parallel based on the outputs of two or more enzyme-biocatalyzed reactions performed in parallel. The signal processing unit can be adapted to identify a type of an injury based on at least one of the generated digital binary output signals. The biochemical logic sensing system can include a monitoring device connected to the signal processing unit, the monitoring device adapted to monitor the output of the enzyme-biocatalyzed reaction. The monitoring device can include an optical monitor, an electrochemical monitor or a combination of optical and electrochemical monitors.

Implementations can optionally include one or more of the following features. The biomarker inputs can include lactate, norepinephrine and glucose. The network of enzyme-biocatalyzed logic gates can include an AND gate and an IDENTITY gate adapted to operate in concert using lactate oxidase, horseradish peroxidase and glucose dehydrogenase enzymes. The network of enzyme-biocatalyzed logic gates can be adapted to process different patterns of the biomarker inputs to generate the output of the enzyme-biocatalyzed reaction comprising norepiquinone and reduced dipotassium salt (NADH). The network of enzyme-biocatalyzed logic gates can be adapted to process the biomarker inputs to provide diagnostic information on multiple conditions at a point-of-care. The network of enzyme-biocatalyzed logic gates can be adapted to process the biomarker inputs to provide diagnostic information on a cardiovascular event. The network of enzyme-biocatalyzed logic gates can be adapted to process the biomarker inputs comprising myeloperoxidase (MPO), lactate dehydrogenase (LDH) and creatine kinase (CK), and generate the output signals comprising reduced dipotassium salt (NADH) and oxidized redox mediator (Mox). The signal processing unit can be adapted to process the output of the enzyme-biocatalyzed reaction comprising reduced dipotassium salt (NADH) and oxidized redox mediator (Mox) to generate the digital binary output that indicates at least one of four possible symptoms comprising a healthy heart (0,0), unhealthy body tissue (1,0), a heart related illness different from Acute myocardial infarction (AMI) (0,1) and AMI (1,1).

Implementations can optionally include one or more of the following features. The network of enzyme-biocatalyzed logic gates can be adapted to analyze the biomarker inputs that are characteristic of at least one of liver injury (LI), soft tissue injury (STI) and abdominal trauma (ABT). The signal processing unit can be adapted to use the generated the digital binary output to produce an alert-type optical output signal in the form of YES-NO separated by a threshold value. The network of enzyme-biocatalyzed logic gates can include an AND gate, an OR gate, a NAND gate, a NOR gate, an XOR gates, or a combination of these logic gates. The network of enzyme-biocatalyzed logic gates can be adapted to process the biomarker inputs comprising alanine transaminase (ALT) and lactate dehydrogenase (LDH) associated with liver injury. The network of enzyme-biocatalyzed logic gates can be adapted to process the biomarker inputs comprising CK and LDH associated with soft tissue injury. The network of enzyme-biocatalyzed logic gates can be adapted to process the biomarker inputs comprising enzyme LDH and its substrate Lac at elevated concentrations associated with abdominal trauma (ABT).

Implementations can optionally include one or more of the following features. The network of enzyme-biocatalyzed logic gates can be adapted to process simultaneously different combinations of five biomarker inputs characteristic of traumatic brain injury (TBI) and soft tissue injury (STI). Three of the biomarker inputs can include creatine kinase (CK), lactate dehydrogenase (LDH) and lactate (Lac) associated with physiological conditions characteristic of STI. Two of the biomarker inputs can include enolase (EN) and glutamate (Glu) associated with the TBI diagnosis. The network of enzyme-biocatalyzed logic gates can be adapted to switch between an STI detection mode and a TBI detection mode. The network of enzyme-biocatalyzed logic gates can be adapted to process simultaneous presence of elevated levels of CK and LDH to trigger a positive diagnosis of STI.

Implementations can optionally include one or more of the following features. The biochemical logic sensing system can include a disposable electrode to obtain a blood sample. The disposable electrode can include a flexible carbon screen-printed electrode (SPE). The network of enzyme-biocatalyzed logic gates can be adapted to assess the biomarker inputs including two or more selected from creatine kinase, lactate dehydrogenase, norepinephrine, glutamate, alanine transaminase, lactate, glucose, glutathione disulfide, and glutathione reductase to assess associated one of soft-tissue injury, traumatic brain injury, liver injury, abdominal trauma, hemorrhagic shock, and oxidative stress. The signal processing unit can be adapted to generate a digitally-encoded multi-bit binary word that represents an injury code. The network of enzyme-biocatalyzed logic gates can be arranged in parallel and multiplexed. The network of enzyme-biocatalyzed logic gates can be adapted to form different arrangements with each enzyme-biocatalyzed logic gate receiving two of the biomarker inputs. The network of enzyme-biocatalyzed logic gates are adapted to process the biomarker inputs that includes Glutathione disulfide (GSSG) and glucocorticoid receptor (GR) associated with oxidative stress. The network of enzyme-biocatalyzed logic gates can be adapted to process the biomarker inputs comprising alanine transaminase (ALT) and lactate dehydrogenase (LDH) to generate NAD⁺ output signal. The biochemical logic sensing system can include a switchable polymer-modified electrode connected to the signal processing unit. The switchable polymer-modified electrode can be adapted convert the generated NAD⁺ output signal to a change in pH.

Implementations can optionally include one or more of the following features. The network of enzyme-biocatalyzed logic gates can include a first AND logic gate activated by lactate dehydrogenase (LDH) and lactate—jointly representing biomarker inputs for abdominal trauma (ABT). A second AND logic gate can be activated by glutathione reductase (GR) and glutathione disulfide (GSSG) representative of oxidative stress when their concentrations are elevated. The network of enzyme-biocatalyzed logic gates can be adapted to process the biomarker inputs comprising α-amylase (Amy) and lactate dehydrogenase (LDH) to analyze radiation-caused tissue damage. The network of enzyme-biocatalyzed logic gates can be adapted to process the biomarker inputs to detect the presence of at least one of explosive compounds and nerve agents.

Implementations can optionally include one or more of the following features. The network of enzyme-biocatalyzed logic gates can be adapted to process the biomarker inputs that comprises 2,4,6-trinitrotoluene (TNT) and paraoxon (PAX) to detection 2,4-dinitrotoluene (DNT) explosive and methyl-parathion (MPT) nerve agent. The network of enzyme-biocatalyzed logic gates can be adapted to utilize enzymes comprising zanitroreductase (NRd), horseradish peroxidase (HRP), acetylcholinesterase (AChE), and choline oxidase (ChOx).

Implementations can optionally include one or more of the following features. The network of enzyme-biocatalyzed logic gates is adapted to simultaneously process biochemically coupled diabetes biomarker inputs comprising glucose and hemoglobin A1c (HbA1c) to generate the output of the enzyme-biocatalyzed reaction; and the signal processing unit can be adapted to process the generated output of the enzyme-biocatalyzed reaction to generate the digital binary output indicative of diabetes. The network of enzyme-biocatalyzed logic gates can be disposed on a single use test strip. The single use test strip can include a reporter anti-HbA1c polyclonal antibody labeled with a horseradish peroxidase (HRP), which is deposited in a sampling zone patch. The single use test strip can also include a capture anti-HbA1c analyte-specific polyclonal antibody immobilized on a surface of a detection zone together with glucose oxidase (GOx) and dried redox dye. The single use test strip can be adapted to receive a liquid sample comprising glucose onto the sample zone to solubilize the labeled reporter antibody, which binds to the HbA1c to for an analyte-antibody complex. When the analyte-antibody complex flows with the liquid sample laterally along the surface of the test strip over the detection zone, the analyte-antibody complex binds to the capture antibody to accumulate at the capture zone of the test strip. The immobilized GOx converts glucose present in the sample to hydrogen peroxide, which serves as a substrate for the HRP-labeled reporter antibody present on the HbA1c molecule.

In another aspect, described is a method of detecting a medical condition using a biochemical sensing system. The method can include operating a network of enzyme-biocatalyzed logic gates in a biochemical sensing system to perform an enzyme-biocatalyzed reaction resembling a Boolean logic operation on biomarker inputs to generate an output of the enzyme-biocatalyzed reaction. The method can include operating a signal processing unit in the biochemical sensing system to generate a digital binary output based on the generated output of the enzyme-biocatalyzed reaction, wherein the digital binary output indicates presence or absence of a medical condition.

Implementations can optionally include one or more of the following features. The method can include operating a drug deliver device to determine a dosage of a drug to deliver based on the digital binary output from the signal processing unit. The method can include operating a monitoring device to analyze the output of the enzyme-biocatalyzed reaction. The method can include operating the network of enzyme-biocatalyzed logic gates to process the biomarker inputs that includes one or a combination of multiple biomarkers associated with a particular type of a medical condition. The method can include operating the network of enzyme-biocatalyzed logic gates to perform enzyme-biocatalyzed reactions in parallel and generate outputs of the enzyme-biocatalyzed reactions in parallel. The method can include operating the signal processing unit to generate digital binary outputs in parallel in response to the parallelly generated outputs of the enzyme-biocatalyzed reactions to detect multiple medical conditions. The method can include operating the signal processing unit to generate an injury code comprising a multi-bit digital word. The method can include operating the network of enzyme-biocatalyzed logic gates to process multiple biomarker inputs associated with a given medical condition. The method can include operating the network of enzyme-biocatalyzed logic gates to switch between multiple modes of operation.

The subject matter described in this specification potentially can provide one or more of the following advantages. For example, the described techniques, apparatus and systems can utilize logic assessment of the cardiovascular problems and disorders. Use of enzyme-logic based diagnosis uses patterns of multiple markers and biochemical processing of the signal information for reliably identifying cardiac abnormalities and providing a final digital answer. Using enzyme-logic based diagnosis can result in a high-fidelity rapid point-of-care diagnosis of acute cardiac events that can lead to greatly increased survival rates. Such accurate diagnosis can also reduce unnecessary hospital admissions or discharges and reduce related health care costs.

In addition, the described techniques, apparatus and systems can be used to provide immediate and reliable diagnosis regarding unpredicted cardiovascular events and to identify those patients experiencing such events, along with the level of risk involved. Implementations of the described techniques, apparatus and systems can provide timely and cost effective diagnose of patients who arrive at the hospital with complaints of chest pain or related warning signs. Rapid and reliable diagnosis of physiological conditions for injured civilians or soldiers on an accident spot or in the battlefield can allow immediate medical intervention prior to hospital evaluation and treatment, potentially leading to decreased mortality and severity of the post-traumatic conditions. The described high fidelity real-time diagnostics can potentially provide reliable decision-making and optimal treatment. The described reliable diagnosis of the type of injury can be coupled to a drug-delivering feedback-loop, leading to a timely therapeutic intervention and hence to a dramatic improvement in the injury outcome.

Also, the described techniques, apparatus and systems can be used to optimize the biocatalytic cascades, and minimize the signal-noise amplification by implementing digital biosensors with built-in logic. In addition, the multi-component biomolecular systems can be transitioned from an in vitro test-tube analysis where they operate in a clean model solution to real-life biomedical samples with many different interferants present.

The described biocatalytic cascades system can be used to establish a decision threshold and Boolean-format digital processing of the inputs that can discard the noise arising from the presence of undesired biomarkers and extracting the signal of interest. Moreover, the described techniques can be used to reduce or eliminate misdiagnosis. Also, the high dynamic range of the logic gate can allow the establishment of an unambiguous decision threshold and digital manipulation of the biomarker signals, thereby alleviating extraneous physiological effects, which allowed the system an enhanced ability to detect injury when compared with traditional biosensing concepts.

### BRIEF DESCRIPTION OF THE DRAWINGS

FIG. 1 shows an exemplary biochemical system for logically analyzing multiple chemical signals.

FIG. 2A shows an analysis of the concentration levels of NE and oxygen performing AND logic operation on two biochemical signals using tyrosinase (10 units mL⁻¹) as the biocatalyst.

FIG. 2B shows formation of NQ detected by optical means using the characteristic absorbance band that was found to be centered at the wavelength 465 nm.

FIG. 2C shows exemplary optical measurements of the NQ formation performed upon application of four different combinations of the input signals (NE and O2): 0, 0; 0, 1; 1, 0 and 1, 1; only the last combination 1, 1 resulted in the formation of the spectrum characteristic of NQ.

FIG. 2D shows the difference between 0 and 1 output signals being even more pronounced upon recording the kinetics of the spectral changes.

FIG. 3A displays amperometric signals corresponding to the in situ production of NQ obtained for different combinations of the input signals.

FIG. 3B shows that the features of the system mimic an AND logic gate, where the output signal is defined as 1 when the current is greater than the threshold value of 12 nA; otherwise, the output signal is considered as a logic 0 value.

FIG. 4A shows a second enzyme logic pathway based on the concerted operation of glucose oxidase (GOx, 0.02 units mL⁻¹), microperoxidase-11 (Mp-11, 0.25 μM) and lactate dehydrogenase (LDH, 20 units mL⁻¹) mimicking an XOR (eXclusive OR) logic gate.

FIG. 4B shows that the output signal produced by the XOR logic gate is defined as the change of the NADH absorbance measured at λ=340 nm [24], reflecting changes of the NADH/NAD+ ratio upon the enzymatic reactions.

FIG. 4C show that the difference between 0, +1 and −1 output signals is even more obvious when the kinetics of the spectral changes is recorded.

FIGS. 5A and 5B show current signals and bar diagram corresponding to a condition when the background current reaches a steady-state value, the measurement proceeded by adding different levels of the glucose and lactate inputs to the enzyme gate solution.

FIGS. 6A and 6B are tables that show the physiological conditions in the model system based on the comparison of two output signals generated by the AND and XOR gates.

FIG. 7A shows exemplary enzyme logic circuitry analyzing three chemical input signals (glucose, lactate and NE) in two parallel-functioning biocatalytic pathways using AND/IDENTITY logic gates.

FIG. 7B shows a scheme where an AND logic gate includes LOx and HRP activated by the lactate and NE input signals and produced norepi-quinone (NQ) as an output signal.

FIG. 7C shows a scheme where the IDENTITY gate is based on GDH which translated the glucose input signal into a NADH output signal.

FIG. 8A shows a formation of NQ was detected by optical absorbance measurements at λ=465 nm.

FIG. 8B shows electrochemical current measurements at the potential of −0.25V.

FIGS. 9A and 9B shows the input of glucose applied at two different levels of 4 mM and 26 mM being considered as logic 0 and 1 values, respectively.

FIGS. 10A and 10B show the optical and electrochemical detections for the output signal produced by the AND gate in the form of NQ.

FIG. 10C summarizes the output optical and electrochemical signals for all combinations of the input signals.

FIGS. 11A and 11B show the optical and electrochemical detection for the output signal produced by the IDENTITY gate in the form of NADH.

FIG. 11C summarizes the output signals obtained by both methods for all combinations of the input signals.

FIG. 12 is a table showing the physiological conditions in an exemplary system based on the comparison of the two output signals generated by the AND/IDENTITY gates.

FIG. 13 shows an example of a enzyme-logic based cardiac sensor system (Cardio-Logic) for reliable and rapid point-of-care diagnostics of cardiac events.

FIG. 14 shows an exemplary circuit diagram of the cardio-logic system.

FIG. 15 shows an example of a biocomputing network diagram showing the built-in logic operations performed by the concatenated enzyme-based gates of the biochemical pathway of FIG. 13.

FIG. 16 is a table that shows a truth table for the logic processing of the biochemical signals of FIG. 14: (OK—healthy heart; U—unhealthy body tissue; H—heart-related condition different from AMI; AMI—acute myocardial infarction).

FIG. 17 shows a general concept of the “Sense & Treat” approach based on enzyme-logic gates processing of the biochemical information regarding the nature and severity of injury and initiating automated treatment by activating a drug-releasing chemical system.

FIG. 18 shows an exemplary ‘Sense-and-Act’ system for the analysis of battlefield injury conditions followed by an automatic medical treatment.

FIG. 19 is a table showing physiological (logic input 0) and pathological (logic input 1) levels of clinically-relevant biomarkers for each logic gate with the output compound indicated.

FIG. 20A shows an exemplary logic analysis of the liver injury (LI) conditions with the biocatalytic cascade applied for the realization of the NAND logic gate activated by ALT (Input 1—corresponding to severe LI) and LDH (Input 2).

FIG. 20B shows an exemplary logic analysis of the liver injury (LI) conditions that shows absorbance changes corresponding to the consumption of NADH upon operation of the logic system in the presence of different combinations of the input signals.

FIG. 20C shows an exemplary logic analysis of the liver injury (LI) conditions with a bar chart showing the NADH absorbance at 340 nm after 20 sec of the biocatalytic reaction activated with different combinations of the biomarker-input signals.

FIG. 21A shows an exemplary logic analysis of the liver injury (LI) conditions with the biocatalytic cascade applied for the realization of the NAND logic gate activated by ALT (Input 1—corresponding to mild LI) and LDH (Input 2).

FIG. 21B shows an exemplary logic analysis of the LI conditions with absorbance changes corresponding to the consumption of NADH upon operation of the logic system in the presence of different combinations of the input signals.

FIG. 21C shows an exemplary logic analysis of the LI conditions with a bar chart showing the NADH absorbance after 70 sec of the biocatalytic reaction activated with different combinations of the biomarker-input signals.

FIG. 22A shows an exemplary logic analysis of the soft tissue injury (STI) conditions with the biocatalytic cascade applied for the realization of the NAND logic gate activated by CK (Input 1) and LDH (Input 2).

FIG. 22B shows an exemplary logic analysis of the STI with absorbance changes corresponding to the consumption of NADH upon operation of the logic system in the presence of different combinations of the input signals.

FIG. 22C shows an exemplary logic analysis of the STI with a bar chart showing the NADH absorbance after 350 sec of the biocatalytic reaction activated with different combinations of the biomarker-input signals.

FIG. 23A shows an exemplary logic analysis of the abdominal trauma (ABT) conditions with the biocatalytic reaction applied for the realization of the AND logic gate activated by Lac (Input 1) and LDH (Input 2).

FIG. 23B shows an exemplary logic analysis of the ABT conditions with absorbance changes corresponding to the formation of NADH upon operation of the logic system in the presence of different combinations of the input signals.

FIG. 23C shows an exemplary logic analysis of the ABT conditions with a bar chart showing the NADH absorbance after 300 sec of the biocatalytic reaction activated with different combinations of the biomarker-input signals.

FIG. 24 shows an exemplary multi-enzyme biocatalytic cascade for the analysis of STI and TBI.

FIG. 25A shows an equivalent logic scheme for exemplary concatenated logic gates operating in STI mode corresponding to the biocatalytic cascade shown in FIG. 24.

FIG. 25B shows an equivalent logic scheme for exemplary concatenated logic gates operating in TBI analysis mode.

FIG. 26 shows a table of concentrations of the inputs activating the biocatalytic cascade for the logic analysis of STI and TBI.

FIG. 27A shows optical detection of the output signal (NADH) generated by the logic system operating for the STI analysis obtained upon different combinations of the injury biomarker input signals (CK, Lac, LDH).

FIG. 27B is a bar chart for the output signals generated by the enzyme logic system for the analysis of STI at 800 seconds.

FIG. 28A shows an exemplary optical detection of the output signal (TMBₒₓ) generated by the logic system operating for the TBI analysis obtained upon different combinations of the biomarker input signals (EN, Glu).

FIG. 28B shows a bar chart for the output signals generated by the enzyme logic system for the analysis of TBI at 800 seconds.

FIG. 29A shows an exemplary biocatalytic cascade instigated by CK and LDH with the level of NADH as an indicator of NAND operation.

FIG. 29B shows an equivalent logic system for the exemplary biocatalytic cascade of FIG. 29A.

FIG. 29C shows an exemplary corresponding truth table with biomedical conclusions drawn from the combinations of the input signals.

FIG. 30A shows exemplary time-dependent optical signals corresponding to the consumption of NADH upon the application of various combinations of the CK and LDH input signals at pH 7.40.

FIG. 30B shows exemplary time-dependent optical signals corresponding to the consumption of NADH upon the application of various combinations of the CK and LDH input signals at pH 7.95.

FIG. 30C exemplary time-dependent optical signals corresponding to the consumption of NADH upon the application of various combinations of the CK and LDH input signals at pH 8.50.

FIG. 31A shows exemplary chronoamperometric curves generated by the NAND gate upon application of various combinations of the CK and LDH input signals at pH 7.95.

FIG. 31B shows an exemplary histogram featuring the NAND logic operation for the corresponding combinations of input signals.

FIG. 32A shows exemplary chronoamperometric curves generated by the NAND gate upon application of various combinations of the input signals with physiological levels of 6 mM LAC, 40 μM PYR, 150 μM PCr, 130 nM ADP, and 30 μM NAD⁺ at pH 7.95.

FIG. 32B shows a histogram featuring the NAND logic operation for the corresponding combinations of input signals.

FIG. 33 is a table of enzyme cascades, equivalent logic gates, and truth tables corresponding to six unique injuries: STI, TBI, LI, ABT, HS, and OS.

FIG. 34A shows optical (left) and electrochemical (right) responses generated by an STI NAND gate upon various combinations of the input biomarkers (0,0), (0,1), (1,0), and (1,1).

FIG. 34B shows optical (left) and electrochemical (right) responses generated by a TBI AND gate upon various combinations of the input biomarkers (0,0), (0,1), (1,0), and (1,1).

FIG. 34C shows optical (left) and electrochemical (right) responses generated by an LI NAND gate upon various combinations of the input biomarkers (0,0), (0,1), (1,0), and (1,1).

FIG. 34D shows optical (left) and electrochemical (right) response generated by an ABT AND gate upon various combinations of the input biomarkers (0,0), (0,1), (1,0), and (1,1).

FIG. 34E shows optical (left) and electrochemical (right) responses generated by an HS AND gate upon various combinations of the input biomarkers (0,0), (0,1), (1,0), and (1,1).

FIG. 34F shows optical (left) and electrochemical (right) responses generated by an OS AND logic gate upon various combinations of the input biomarkers (0,0), (0,1), (1,0), and (1,1).

FIG. 35A shows optical (left) and electrochemical (right) bar charts obtained by sampling the output of an STI NAND logic gates upon various combinations of the input biomarkers (0,0), (0,1), (1,0), and (1,1).

FIG. 35B shows optical (left) and electrochemical (right) bar charts obtained by sampling the output of a TBI AND logic gates upon various combinations of the input biomarkers (0,0), (0,1), (1,0), and (1,1).

FIG. 35C shows optical (left) and electrochemical (right) bar charts obtained by sampling the output of an LI NAND logic gates upon various combinations of the input biomarkers (0,0), (0,1), (1,0), and (1,1).

FIG. 35D shows optical (left) and electrochemical (right) bar charts obtained by sampling the output of an ABT AND logic gates upon various combinations of the input biomarkers (0,0), (0,1), (1,0), and (1,1).

FIG. 35E shows optical (left) and electrochemical (right) bar charts obtained by sampling the output of an HS AND logic gates upon various combinations of the input biomarkers (0,0), (0,1), (1,0), and (1,1).

FIG. 35F shows optical (left) and electrochemical (right) bar charts obtained by sampling the output of an OS AND logic gates upon various combinations of the input biomarkers (0,0), (0,1), (1,0), and (1,1).

FIG. 36 is a truth table and corresponding injury codes for all 64 possible combinations of STI, TBI, LI, ABT, HS, and OS.

FIG. 37 is an exemplary table of physiological and pathological levels of clinically-relevant biomarkers for each logic gate with the output compound indicated.

FIG. 38 is an exemplary biocatalytic cascade used for the logic processing of the chemical input signals, resulting in situ pH changes and activation of the electrode interface.

FIG. 39 shows variation of the anodic peak current measured by cyclic voltammetry upon titration of the sensing system to different pH values. Cyclic voltammograms were recorded after allowing 10 min for the working electrode to equilibrate at each pH. The system composition is specified in the Experimental Section.

FIG. 40 shows pH Changes generated in situ by the biocatalytic cascade activated with various combinations of the two chemical input signals, ALT, LDH: a) 0,0; b) 0,1; c) 1,0; d) 1,1. The dotted line corresponds to the pKₐ value of the P4VP-brush obtained from the titration curve in FIG. 1. The system composition is specified in the Experimental Section.

FIG. 41 shows exemplary cyclic voltammograms obtained for the ITO electrode modified with the P4VP-polymer brush in: a) the initial OFF state at t=1 min after the initiation of the biocatalytic cascade, pH 6.3, and b) the ON state enabled by the ALT, LDH input combination 1,1, at t=80 min, pH 4.75. The system composition is specified in the Experimental Section.

FIG. 42 shows a sample impedance spectra in the form of Nyquist plots for: a) the OFF state of the electrode, with the initial pH 6.6, electron transfer resistance, Rₑₜ, ca. 40 kΩ, and b) the ON state enabled by the input combination 1,1, with the pH 4.76 generated in situ, Rₑₜ, ca. 60.3Ω. (Note different scales for curves a and b). The system composition is specified in the Experimental Section.

FIG. 43 shows an exemplary lactate/LDH biocatalytic cascade and its AND logic gate equivalent. MGₒₓ and MG_{red} are the oxidized and reduced forms of MG, respectively.

FIG. 44 shows an exemplary GSSG/GR biocatalytic cascade and its AND logic gate equivalent. CoPCₒₓ and CoPC_{red} are the oxidized and reduced forms of CoPC, respectively.

FIG. 45A shows exemplary optical absorbance (λ=340 nm) of the NADH output generated in situ by the lactate/LDH biocatalytic cascade upon different combinations of the input signals: a) 0,0; b) 0,1; c) 1,0 and d) 1,1.

FIG. 45B shows exemplary electrochemical amperometric (0.1 V) detection of the NADH output generated in situ by the lactate/LDH biocatalytic cascade upon different combinations of the input signals: a) 0,0; b) 0,1; c) 1,0 and d) 1,1.

FIG. 45C shows an exemplary truth table corresponding to the AND logic function of the system.

FIGS. 46A, 46B and 46C show experimental data and their analysis for exemplary optical measurements.

FIGS. 46D, 46E and 46F show ex electrochemical measurements.

FIG. 47 shows normalized output logic values z for four logic inputs, for the different measurement techniques for the LDH-based AND gate. The logic gate composition and the input concentrations corresponding to the logic 0 and 1 values are specified in the Experimental Section.

FIGS. 48A and 48B show noise propagation properties of the LDH-based AND gate, as calculated from fits of exemplary optical data.

FIGS. 48C and 48D show noise propagation properties of the LDH-based AND gate, as calculated from fits of exemplary electrochemical data.

FIG. 49A shows exemplary optical absorbance (λ=412 nm) of the GSH output generated in situ by the GSSG/GR biocatalytic cascade upon different combinations of the input signals: a) 0,0; b) 0,1; c) 1,0 and d) 1,1.

FIG. 49B shows exemplary electrochemical chronoamperometric (0.5 V) detection of the GSH output generated in situ by the GSSG/GR biocatalytic cascade upon different combinations of the input signals: a) 0,0; b) 0,1; c) 1,0 and d) 1,1.

FIG. 49C is an exemplary truth table corresponding to the AND logic operation of the system.

FIG. 50A shows exemplary optical measurements and numerical fitting of the response surface for the GR-based gate: experimental response surface.

FIG. 50B shows exemplary optical measurements and numerical fitting of the response surface for the GR-based gate: numerical fit of the surface in 50A, according to Equation (3), yielding estimates α=1.14·10⁻² (μm)⁻¹ and β=0.00 U⁻¹ L.

FIG. 50C shows exemplary optical measurements and numerical fitting of the response surface for the GR-based gate: logic surface z(x, y).

FIG. 51 shows normalized output logic values z for four logic inputs, for the GR-based gate. The logic gate composition and the input concentrations corresponding to the logic 0 and 1 values are specified in the Experimental Section.

FIGS. 52A and 52B show noise propagation properties of the GR-based AND gate, as calculated from fit of the optical data.

FIG. 53 shows an exemplary multi-enzyme biocatalytic cascade for the analysis of irradiation damage. Biomarkers Amy and LDH are applied as the input signals; NADH is the output signal. Other products of the biocatalytic cascade are the following: glucose (Glc), glucose-6-phosphate (G6P), pyruvate (Pyr) and lactate (Lac). Note that for simplicity the scheme does not include some reacting cofactors, promoters and byproducts—for the full composition of the system refer to the Experimental Section. Schematic representation of the NAND logic gate and the corresponding truth table are shown at the bottom.

FIG. 54 shows an exemplary optical detection of the output signal generated by the logic system obtained upon different combinations of the biomarker input signals (Amy, LDH). Inset: Bar chart for the optical output signals generated by the enzyme logic system obtained after 350 seconds of the biocatalytic reaction. The radiation damage diagnosis corresponds to the output signal below the decision threshold (dashed line). The logic system composition and the input signal concentrations are specified in the Experimental Section.

FIG. 55A shows an exemplary biocatalytic cascade used to perform NOR logic operation in connection to trinitrotoluene (TNT) and paraoxon (PAX) inputs.

FIG. 55B shows an equivalent logic system of FIG. 55A.

FIG. 55C shows a truth table corresponding to FIGS. 55A and 55B with assessment drawn from the combinations of the input signals.

FIG. 56 shows exemplary gate mapping.

FIG. 57A shows exemplary chronoamperograms of the NOR enzyme logic gate corresponding to the [TNT (μg mL−1), PAX (μM)]=(a) [0,0], (b) [0,10], (c) [10,0], and (d) [01,10] inputs for three independent trials.

FIG. 57B is a bar chart comparing the magnitude of the response for the four combinations of the inputs.

FIG. 57C shows a bar chart of the NOR gate operating at its limit of detection for the four logic levels (TNT, PAX)=(0,0), (0,1), (1,0), and (1,1) for three independent trials.

FIG. 58A is a process flow diagram outlining an exemplary equivalent functional behavior of the microelectronic sensing system.

FIG. 58B is an exemplary circuit-level schematic of the supporting electronics designed for the analysis of abdominal trauma. In order to realize correct logic operation, the CMOS AND logic gate in the figure is replaced with a CMOS NAND logic gate for the readout of the soft tissue injury system.

FIG. 59A is an image of an exemplary microelectronic system (US 1¢ and screen printed three-electrode strip shown for size comparison).

FIG. 59B is an image of an obverse detail of an exemplary microelectronic system indicating the locations of the constituent components on the PCB.

FIG. 59C is an image of a reverse detail of an exemplary microelectronic system indicating the locations of the constituent components on the PCB.

FIG. 60A shows an exemplary biocatalytic cascade instigated by creatine kinase (CK) and lactate dehydrogenase (LDH) emulating NAND operation.

FIG. 60B is a block diagram showing an exemplary equivalent logic system.

FIG. 60C is a corresponding truth table with biomedical conclusions, drawn from the combinations of the input signals.

FIG. 61A is a bar chart featuring the NAND logic operation for the corresponding combinations of input signals. Electrochemical measurements were performed at E=0.0 V vs. Ag/AgCl. Dashed lines indicate the decision threshold for the realization of NAND gate operation.

FIG. 61B is images of an exemplary microelectronic system under the application of various combinations of the input biomarkers CK and LDH. Only the pathological scenario involving high levels of both CK and LDH corresponding to the (1,1) logic level rendered an output logic 0, resulting in the illumination of an LED.

FIG. 62A shows an exemplary biocatalytic cascade instigated by lactate (LAC) and lactate dehydrogenase (LDH) emulating AND operation.

FIG. 62B shows an exemplary equivalent logic system.

FIG. 62C shows a corresponding truth table with biomedical conclusions, drawn from the combinations of the input signals.

FIG. 63A is a bar chart featuring an exemplary AND logic operation for the corresponding combinations of input signals. Electrochemical measurements were performed at E=0.0 V vs. Ag/AgCl. Dashed lines indicate the decision threshold for the realization of AND gate operation.

FIG. 63B shows images of an exemplary microelectronic system under the application of various combinations of the input biomarkers LAC and LDH. Only the pathological scenario involving high levels of both LAC and LDH corresponding to the (1,1) logic level rendered an output logic 1, resulting in the illumination of an LED.

FIG. 64 shows a schematic performance of an exemplary two-marker strip based on the immuno-recognition followed by biocatalytic process.

FIGS. 65A, 65B, 65C, 65D, 65E, 65F and 65G are process flow diagrams of an exemplary process for implementing enzyme-logic based diagnosis.

Like reference symbols and designations in the various drawings indicate like elements.

### DETAILED DESCRIPTION

The techniques, systems and apparatus described in this application can be used to implement biochemical logic systems to evaluate and diagnose patients. The described biochemical logic system can include an enzyme logic sensing system for a reliable assessment of the overall physiological condition associated with an injury or illness. The biochemical logic system described in this document can be implemented to include various networks of enzyme-biocatalyzed logic gates arranged in different arrangements to process one or a combination of biomarker inputs associated with one or more medical conditions or injuries. The various networks or enzyme-biocatalyzed logic gates can include different combinations of logic gates selected from an AND gate, an OR gate, an XOR gate, a NAND gate, an IDENTITY gate, etc. These logic gates can be mixed and matched to provide various combinations that process multiple biomarkers. Some implementations can allow parallel processing of different biomarker inputs.

Enzyme Logic Gates for Assessing Physiological Conditions During an Injury: Towards Digital Sensors and Actuators

While important for accident-related civilian injuries, immediate assessment of physiological conditions followed by a timely optimal therapeutic treatment is particularly needed for injured soldiers, when rapid evacuation to a hospital is not always possible. Since the majority of battlefield, criminal or traffic deaths occur within the first 30 min after injury, rapid diagnosis and treatment are crucial for enhancing the survival rate. Various injury scenarios (brain injury, trauma, shock, fatigue, etc.) can call for different therapeutic interventions.

In one aspect, an enzyme logic sensing system is described for a reliable assessment of the overall physiological condition during an injury. An exemplary enzyme logic sensing system can include AND and XOR logic gates based on the concerted operation of tyrosinase, glucose oxidase, lactate dehydrogenase and microperoxidase-11. The enzyme logic system can be activated by physiologically relevant concentrations of the input signals of biomarkers including norepinephrine, oxygen, glucose and lactate mimicking in vitro different injuries, including trauma brain injury and hemorrhagic shock. Such biochemical logic gates can facilitate decision-making towards the initiation of an optimal timely therapeutic intervention, leading to improved survival of injured soldiers and civilians. The biochemical logic system analyzing biomarker signals represents the first step towards digitally operating sensors and actuators with chemical information processing steps.

Results are described of an enzyme logic sensing system aimed at distinguishing different types of injuries. Formulation of enzyme-based logic gates and their networks can be provided based on the concept of biochemical information processing (biocomputing), a sub-area of unconventional chemical computing. Chemical logic gates can mimic Boolean logic operations and can include chemical systems, where the input and output signals are represented by concentrations of reactants and products, respectively. Specifically, enzyme-based logic gates can perform enzyme-biocatalyzed reactions resembling properties of Boolean logic systems. Enzyme logic networks can include several concatenated logic gates operating in a concerted way. These systems are capable of processing biochemical information received in the form of various combinations of chemical signals and perform logic operations on them. Biochemical logic systems, such as those described here, can be used to assess the states of vital injury biomarkers and create high fidelity diagnosis for a greatly improved decision-making. By using various injury biomarkers as inputs for the enzyme logic gates and automatically processing of the physiological information, such biochemical logic systems could offer reliable assessment of the overall physiological condition during an injury and would eventually initiate optimal timely therapeutic intervention. Biochemical signals logically processed by enzymes could be coupled with electronic transducers operating as multi-signal-responsive biosensors or with nano-structured signal-responsive materials operating as chemically triggered actuators. Such integrated systems composed of the signal-processing units, electronic transducers and chemical actuators, can allow autonomous timely and reliable analysis of pathophysiological changes originating from an injury, identification of the type of injury, signaling about the medical conditions and performing primary drug-delivery according to the medical needs. Such application of enzyme logic systems (biocomputing units) coupled with signal-responsive materials could allow the analysis, decision-making and therapeutic intervention performed by pure chemical means without sophisticated electronic equipment, leading to improved survival of injured civilians and soldiers.

Logic Gates Processing

To illustrate exemplary implementations of biochemical signal processing and autonomous decision-making, glucose, lactate, norepinephrine (NE) and oxygen were selected as physiological markers signaling on different kinds of injuries. None of them analyzed separately can be considered as a medically meaningful sign of specific injuries; however, all of them measured together can provide enough information to distinguish various types of injuries. Indeed, abnormal increase in glucose concentration might originate from hemorrhagic shock (HS), higher than normal physiological concentration of lactate could be caused also by HS or/and trauma brain injury (TBI), while high concentration of NE signals about any traumatic injury. Lack of oxygen in blood could mean ischemic state or/and heart attack. Logic processing of different combinations of the chemical inputs can reveal specific signal pattern characteristics of various normal/abnormal physiological conditions, thus identifying the specific injury. In the experiments conducted using the enzyme logic gates, Glucose, lactate, NE and oxygen were applied as chemical input signals activating enzyme logic operations. The digitized signals were considered as logic 0 when the inputs were applied at physiologically normal concentrations: 4 mM glucose, 2 mM lactate and 2.2 nMNE. Deoxygenated solutions (achieved by bubbling argon) were considered as 0 input for O₂. Abnormal high concentrations of glucose, 30 mM, characteristic of HS, lactate, 13 mM, observed in case of both TBI and HS, and NE, 3.5 μM, typical for any traumatic injury were considered as logic 1 values for the input signals. Similar to the digitalization of signals used in electronics, the intermediate values of the concentrations of the chemical inputs were considered as undefined signals.

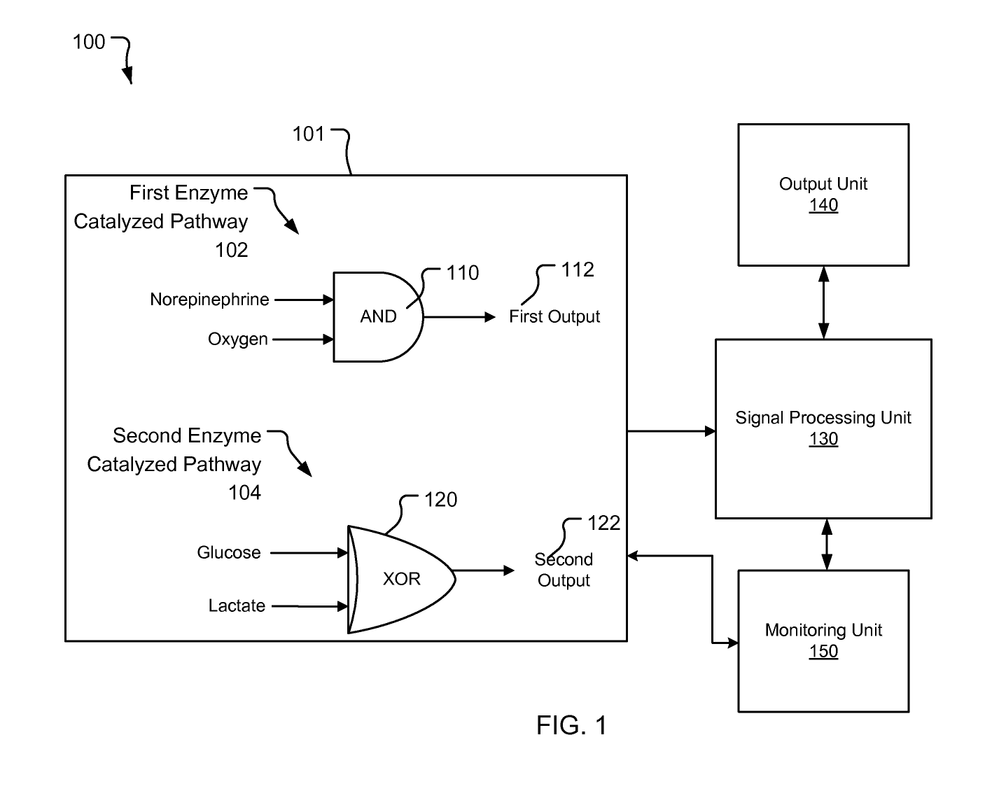

FIG. 1 shows an exemplary biochemical system 100 for logically analyzing multiple chemical signals. A network of enzyme-biocatalyzed logic gates 101 is connected to a signal processing unit 130. The network of enzyme-biocatalyzed logic gates 101 can include various types of logic gates such as an AND gate, an OR gate, an XOR gate, a NAND gate, an IDENTITY gate, etc. connected to form different arrangements, including cascades, concatenations, parallel signal pathways, etc. The signal processing unit 130 processes the outputs (e.g., 112, 122) from the network of enzyme-biocatalyzed logic gates to generate a digital binary output having a value of ‘0’ or ‘1’ to indicate an absence or presence of a medical condition in a simple ‘YES or No’ type of an output. The signal processing unit can be connected to an output unit that provides visual indication of the digital binary output generated by the signal processing unit 130. The output unit 140 can include light emitting diodes (LEDS) arranged to visualize the digital binary output; a display unit, such as an LCD display device or the like. Also, the output device 140 can be a non-electronic device such as a color changing paper, etc.

Also a monitoring unit 150 can be connected to the signal processing unit to optically or electrochemically confirm the presence of the output signals (e.g., 112, 114) as described below. For example, an optical monitoring unit 150 can detect a product of an enzyme-biocatalyzed reaction based on an absorbance measurement. An electrochemical monitor can detect a product of an enzyme-biocatalyzed reaction based on a current measured. The monitoring unit 150 can be connected directly to the network of enzyme-biocatlyzed logic gates directly.

In some implementations, the biochemical system 100 can be implemented on a single device. In some implementation, some of the components of the biochemical system 100 can be implemented as separate sensing unit, processing unit and display unit in communication with each other either wired or wirelessly.

Moreover, for the various aspects and implementations described herein, the various networks of enzyme-biocatalyzed logic gates can be implemented as a part of a biochemical system similar to the one described with respect to FIG. 1.

In the example shown in FIG. 1, the system 100 includes an AND gate 110 and an XOR gate for analyzing four chemical signals in two parallel-operating enzyme-catalyzed pathways 102 and 104. In the first enzyme-catalyzed pathway 102, the AND gate 110 analyzes NE and oxygen to provide a first output 112. In the second enzyme-catalyzed pathway 104, the XOR gate analyzes glucose and lactate to provide a second output 122. In addition, the biochemical system 100 can include mechanisms for optical and electrochemical monitoring of the chemical outputs 112.

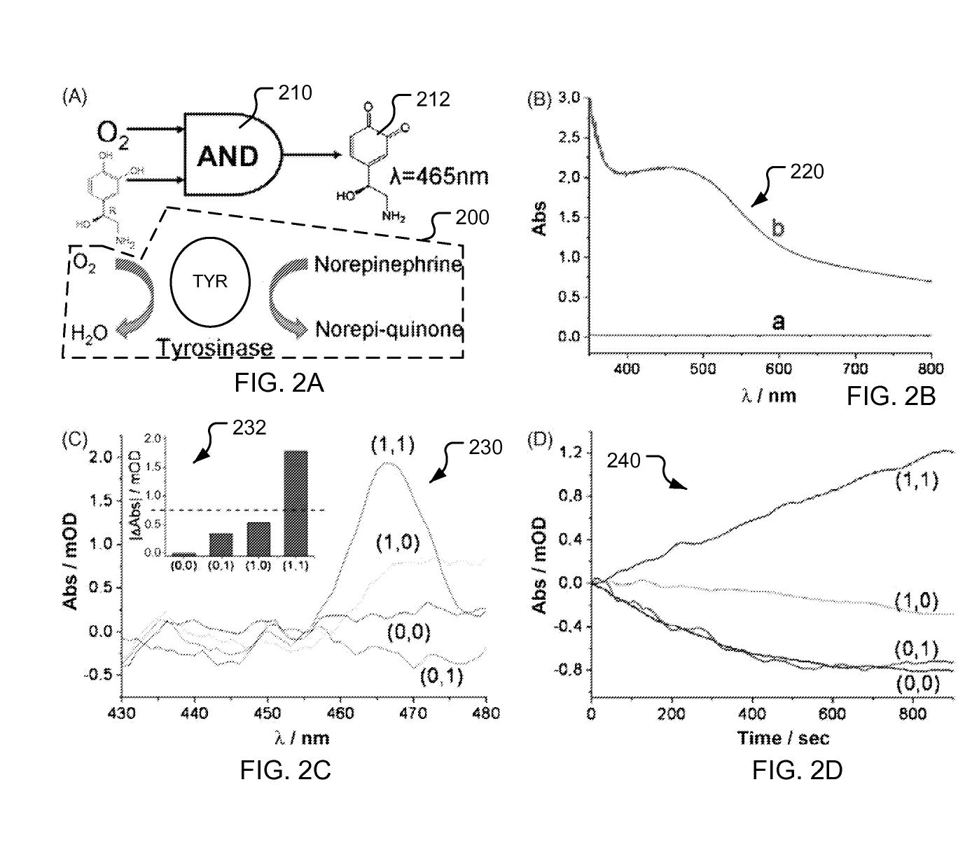

FIG. 2A shows an exemplary biochemical reaction in a first enzyme-catalyzed pathway 200 aimed to analyze concentration levels of NE and oxygen by performing AND logic operation using an AND logic 210 on the two biochemical signals (NE and O₂) using tyrosinase (10 e.g., units mL⁻¹) as the biocatalyst to obtain a first output 212. The first enzyme-catalyzed pathway 200 can be implemented substantially similar to the first enzyme-catalyzed pathway 102 in FIG. 1. Also, the AND gate 210 can be implemented to be substantially similar to the AND logic gate 110 in FIG. 1. Moreover, the output 212 can be substantially similar to the output 112 in FIG. 1.

The biocatalytic oxidation of NE yielding norepi-quinone (NQ) proceeds only in the presence of both substrates: NE and O₂, while lack of any of them results in the inhibition of the biocatalytic reaction. The formation of NQ was detected by optical means using the characteristic absorbance band 220 that was found to be centered at the wavelength 465 nm as shown in FIG. 2B. The optical measurements of the NQ formation (230) were performed upon application of four different combinations of the input signals (NE and O₂): (0, 0); (0, 1); (1, 0) and 1(,1); only the last combination (1, 1) resulted in the formation of the spectrum characteristic of NQ as shown in FIG. 2C. The built up of the absorbance at λ=465 nm above the threshold value of 0.75 mOD was considered as the output 1, signaling the formation of NQ, while the smaller absorbance changes were accepted as 0 output signal. While the spectral measurements were performed at very low absorbance values (note mOD units) originating from the used low physiological concentrations of NE, a clear distinction between 0 and 1 output signals was still observed as shown in the inset 232 of FIG. 2C. The features of this biocatalytic process resemble Boolean AND logic operation. The difference between 0 and 1 output signals is even more pronounced upon recording the kinetics of the spectral changes (240) as shown in FIG. 2D. The time-interval of ca. 300 s is enough to see clearly the formation of the NQ absorbance produced upon application of 1, 1 input signals, compared with the background noise obtained for all other combinations of the inputs.

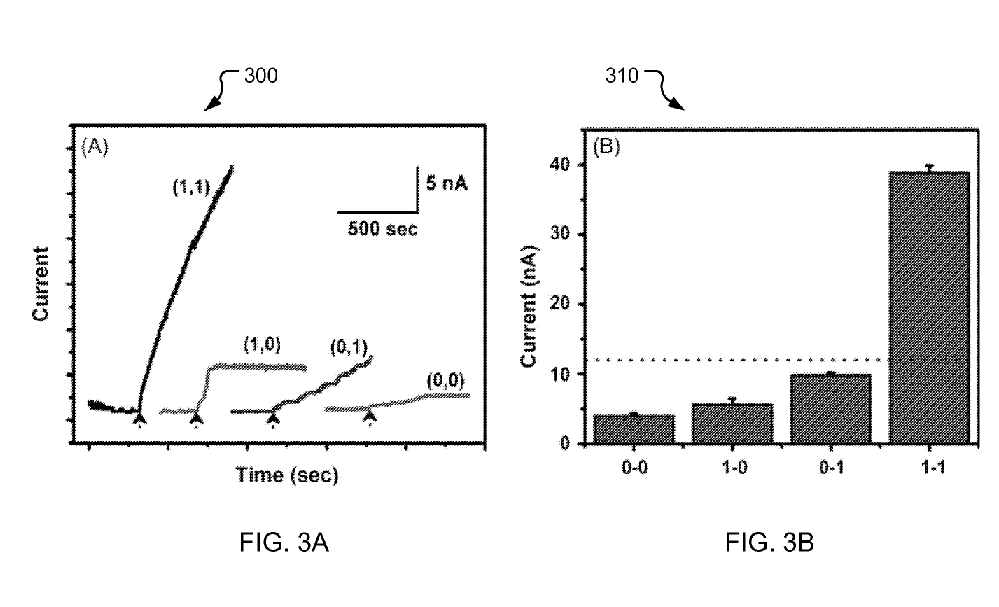

The NE-O₂-dependent AND logic gate was also examined with electrochemical measurements. A defined cathodic peak, corresponding to the reduction of NQ, was clearly observed at −0.291V using square-wave voltammetry. The electrochemical response of NQ produced in situ by the biocatalytic reaction was measured in the presence of O₂ at a potential of −0.30 V. FIG. 3A displays exemplary amperometric signals 300 corresponding to the in situ production of NQ obtained for different combinations of the input signals. Similar to the optical measurements, the current built up was significantly higher upon applying the 1, 1 input signals. FIG. 3B shows exemplary current measurements 310 for the various input signals. The features of the system mimic an AND logic gate, where the output signal is defined as 1 when the current is greater than the threshold value of 12 nA; otherwise, the output signal is considered as a logic 0 value.

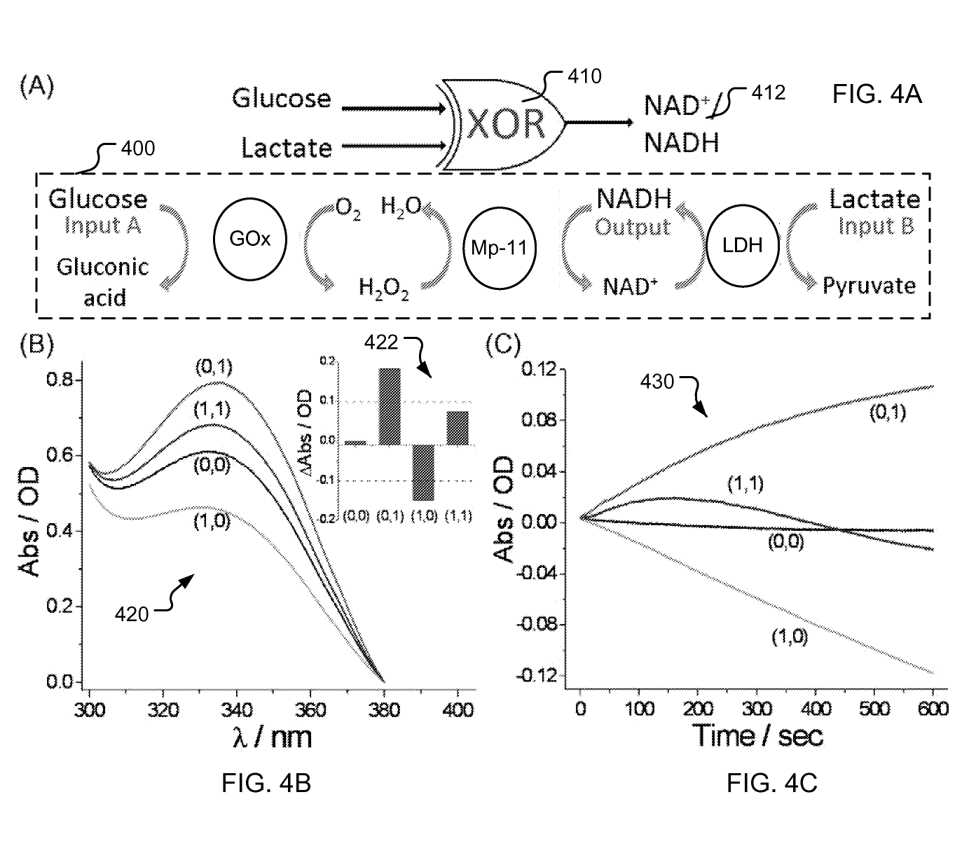

FIG. 4A shows an exemplary second enzyme logic pathway 400 based on the concerted operation of glucose oxidase (GOx, 0.02 units mL⁻¹), microperoxidase-11 (Mp-11, 0.25 μM) and lactate dehydrogenase (LDH, 20 units mL⁻¹) mimicking an XOR (eXclusive OR) logic gate 410 that produces a second output 412. The second enzyme-catalyzed pathway 400 can be implemented substantially similar to the second enzyme-catalyzed pathway 104 in FIG. 1. Also, the XOR gate 410 can be implemented to be substantially similar to the XOR logic gate 120 in FIG. 1. Moreover, the output 412 can be substantially similar to the output 122 in FIG. 1.

The logic XOR gate 410 accepts two independent input signals: glucose and lactate, producing H₂O₂ as an intermediate product and different NADH/NAD⁺ concentration ratios 412 depending on the relative activity of the reductive and oxidative branches of the biocatalytic system. The physiologically normal glucose concentration, 4 mM, was considered as digital 0 input signal, while the increased concentration of glucose, 30 mM, was defined as an input signal 1. The lactate signal was considered as logic 0 for the physiologically normal concentration of 2 mM, while the abnormally increased lactate concentration of 13 mM was used as an input signal 1. The output signal 420 produced by the XOR logic gate 410 was defined as the change of the NADH absorbance measured at λ=340 nm, reflecting changes of the NADH/NAD+ ratio upon the enzymatic reactions as shown in FIG. 4B. The NADH/NAD+ concentration ratio was changed when the reductive and oxidative branches of the biocatalytic system in the XOR gate were unbalanced (input signals 0, 1 and 1, 0), while input signal combinations of 0, 0 and 1, 1 preserved the ratio because of the balance in the reduction and oxidation pathways as shown in the inset 422 of FIG. 4B. The biocatalytic activities of GOx, Mp-11 and LDH were optimized to maintain the steady-state balance for the NADH/NAD+ ratio for the combination of 1, 1 input signals. The features of the logic system resemble the properties of XOR gate, if the absolute value of the absorbance change, |ΔAbs|, is used as the output signal. However, richer information could be obtained from the logic system if the sign of the optical changes is taken into account. This results in +1 output signal when the absorbance is increased above the threshold value of 0.1 OD and −1 output signal when the absorbance is decreased below the threshold value of −0.1 OD, reflecting the increased and decreased ratio of NADH/NAD+, respectively. The difference between 0, +1 and −1 output signals is even more obvious when the kinetics of the spectral changes (430) is recorded as shown in FIG. 4C. The time-interval of ca. 300 s is sufficient for observing clearly the formation of the increasing or decreasing absorbance of NADH produced upon application of 0, 1 and 1, 0 input signals, compared with the balanced steady-state obtained at 1, 1 combination of the input signals.

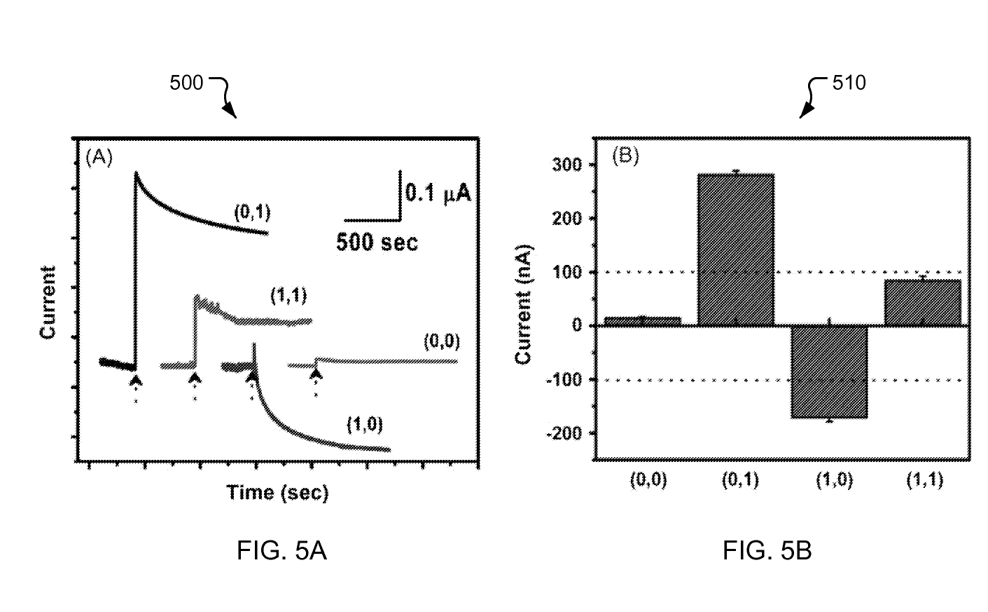

Similarly to the optical measurements, an electrochemical readout from the XOR logic gate was based on the measurements of the NADH concentrations. The exact potential for the oxidation of NADH was determined using square wave voltammetry of NADH (10 μM) in 50 mM phosphate buffer solution (pH 7.2). The voltammogram shows a clear oxidation peak at +0.672V. The amperometric monitoring of the XOR gate was thus carried out at a constant potential of +0.75V in the presence of NADH/NAD+ (0.1 mM/3 mM). When the background current reaches a steady-state value, the measurement proceeded by adding different levels of the glucose and lactate inputs to the enzyme gate solution. The corresponding current signals 500 and bar diagrams 510 are displayed in FIGS. 5A and 5B, respectively. The inputs 0, 0 and 1, 1 provoke small current signals corresponding to a 0 output (based on a 0.10 μA threshold), while the 0, 1 and 1, 0 inputs lead to outputs of +1 and −1, respectively, in connection to a threshold values of ±0.10 μA. These results and their bar diagrams are in strong agreement with the optical results reported above.

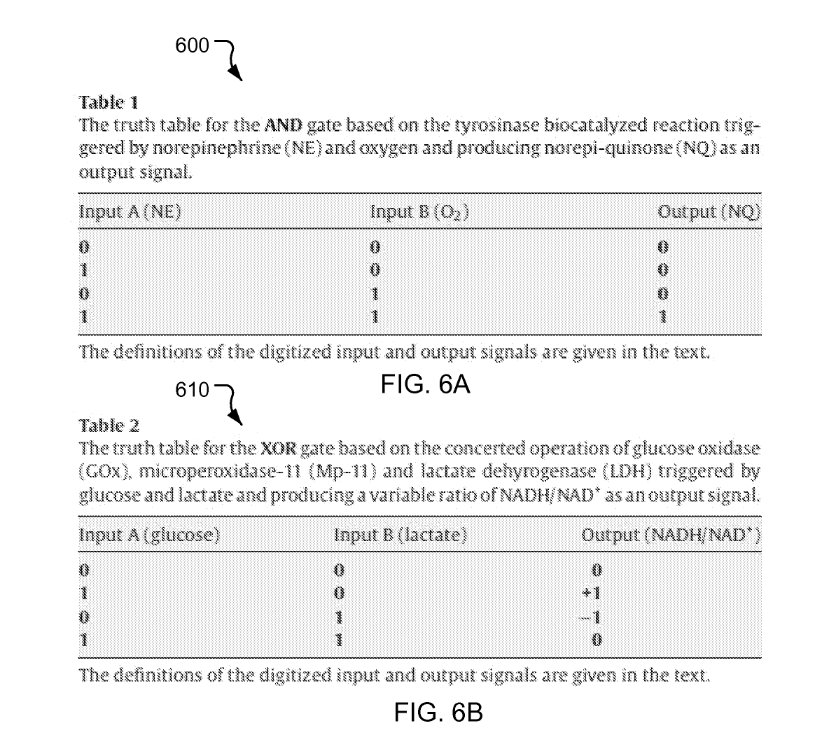

The physiological conditions in the model system can be determined based on the comparison of two output signals generated by the AND and XOR gates as shown in Table 1 (600) of FIG. 6A and Table 2 (610) of FIG. 6B. If the AND gate generated 0 output signal, the final output of the logic network was considered as 0 regardless of the output produced by the XOR gate. This situation could be interpreted as a non-traumatic injury physiological change of glucose and lactate concentrations. In case the AND gate generates 1 output signal the system interprets that there is a traumatic injury proceeding to read the result given by the XOR gate, which can be 0, +1, −1 and again 0. The last 0 output (corresponding to the 1, 1 combination of the input signals) has the physiological meaning that unveils a HS situation, while the +1 output has the physiological match to TBI. The obtained truth tables correspond to the model in vitro conditions. In real physiological conditions the input signals of glucose and lactate for the XOR gate cannot be 0 if there is 1 output signal in the AND gate (conditions of a serious traumatic shock). Thus, the first 0 output signal in Table 2 can not appear if the AND gate generates 1 output signal. Similarly, the combination of the input signals 0 for glucose and 1 for lactate applied to the XOR gate is physiologically impossible (making the −1 output signal of the XOR gate impossible) when there is 1 output signal from the AND gate.

Chemicals and Reagents

All chemicals were purchased and used as supplied without any further purification. From Sigma-Aldrich: β-nicotinamide adenine dinucleotide, reduced dipotassium salt (NADH), >95% purity; β-nicotinamide adenine dinucleotide (NAD+) >95% purity; β-D-(+)-glucose, 99.5% GC; (L)-norepinephrine (NE) ≧98%, crystalline; L-(+)-lactic acid <98%; microperoxidase (Mp-11) sodium salt <90% (HPLC). Enzymes used were glucose oxidase (GOx) from Aspergillus niger type X-S (E.C. 1.1.3.4); L-lactate dehydrogenase (LDH) from porcine heart (E.C. 1.1.1.27); tyrosinase (Tyr) from mushroom (E.C. 1.14.18.1).

Instruments

The absorbance spectra were recorded using a Shimadzu UV-2450PC spectrophotometer. The electrochemical measurements were performed using the CH Instrument Model CHI630C and an electrochemical cell consisting of a glassy carbon (GC) working electrode, Ag/AgCl (3M NaCl) reference and a platinum wire counter electrode.

Logic Gates and Input Signals

In one example, an AND gate as used in the described enzyme logic system can be implemented to include (or in some implementations, consist of) a 1 mL solution of phosphate buffer (50 mM, pH 7.2), containing 10 units/mL of Tyr. The input signals can include NE and oxygen. NE concentrations can be 2.2 nM as 0 input and 3.5 μM as 1 input. Oxygen input can be defined as 1 when it is in equilibrium with the air and 0 when the solution is bubbled with argon for 10 min. The XOR gate can include (or in some implementations, consist of) of a 1 mL solution of phosphate buffer (50 mM, pH 7.2), containing 0.02 units mL⁻¹ GOx, 20 units mL⁻¹ LDH, 0.25 μM Mp-11, 0.1 mM NADH, 3 mM NAD⁺. Glucose concentrations of 4 mM and 30 mM can be defined as digital logic inputs of 0 and 1, respectively. Lactate concentrations of 2 mM and 13 mM can be defined as digital logic inputs of 0 and 1, respectively. In XOR gate experiments oxygen can be in constant natural equilibrium with air. The optical and electrochemical outputs of the AND gate can be monitored at 465 nm and −0.30 V, respectively, while those of the XOR gate can be measured at 340 nm and +0.75 V, respectively.

The above described biochemical system demonstrated the possibility to process biochemical signals at their physiologically relevant concentrations by the enzyme reactions. Stable information processing with clearly distinguishable output signals from different combinations of biochemical input signals, characteristics of various abnormal physiological conditions, can be achieved. In addition the described techniques, systems and apparatus can be used to implement: (i) further increase in the enzyme logic complexity is needed for the future integration of biomedical devices with natural in vivo biochemical pathways; and (ii) directly immobilize the enzyme systems processing biochemical signals on the signal-transducing interfaces. The described techniques, systems and apparatus can be used to implement fully chemical digital biosensors processing biochemical information and producing an output signal dependent on the whole pattern of various input signals. The output signal might be directed to chemical actuators (e.g. signal-responsive membranes) leading to an on-demand drug release. Such biochemical logic gates can facilitate decision-making in connection to integrated autonomous feedback system and hence to revolutionize the monitoring and treatment of injured soldiers and civilians.

Enzyme Logic Gates for the Digital Analysis of Physiological Level Upon Injury

In another aspect, a biocomputing system can be designed to include a combination of AND/IDENTITY logic gates based on the concerted operation of three enzymes: lactate oxidase, horseradish peroxidase and glucose dehydrogenase. The biocomputer system can process biochemical information related to pathophysiological conditions originating from various injuries. Three biochemical markers: lactate, norepinephrine and glucose were applied as input signals to activate the enzyme logic system. Physiologically normal concentrations of the markers were selected as logic 0 values of the input signals, while their abnormally increased concentrations, indicative of various injury conditions were defined as logic 1 input. Biochemical processing of different patterns of the biomarkers resulted in the formation of norepi-quinone and NADH defined as the output signals. Optical and electrochemical means were used to follow the formation of the output signals for eight different combinations of three input signals. The enzymatically processed biochemical information presented in the form of a logic truth table allowed distinguishing the difference between normal physiological conditions, pathophysiological conditions corresponding to traumatic brain injury and hemorrhagic shock, and abnormal situations (not corresponding to injury). The developed system represents a biocomputing logic system applied for the analysis of biomedical conditions related to various injuries. Such biochemical logic gates can facilitate decision-making in connection to an integrated therapeutic feedback-loop system and hence can revolutionize the monitoring and treatment of injured civilians and soldiers.

The concept of biochemical information processing (biocomputing) can be used to analyze various chemical signals, their comparison and processing according to a special chemical built-in “program” using only chemical means without the involvement of electronic computers. Biochemical processing of information with biomolecules and biological components by realizing and networking logic “gates” that mimic Boolean digital logic can have various applications for clinical diagnostics. Indeed, biomolecules are selective and usable in complex biochemical environments. Such systems can also be appropriate for interfacing of the biocomputing “devices” with processes in living organisms, and hence can be useful for future practical biomedical applications. Integration of biocomputing elements with sensing processes could allow multi-signal analysis followed by chemical processing of the data, giving the final answer regarding the physiological conditions of an injured person in a digital (“YES” or “NO”) form. The biocomputing system—analyzing the biochemical information and making decision—could be connected to a chemical actuator releasing an appropriate drug to treat an injured person. Biocomputing elements of even moderate complexity can allow an effective interface between complex physiological systems and nano-structured signal-responsive materials and/or electronic systems. Thus, integration of the biocomputing concepts into sensing technology can contribute to a new emerging technological paradigm: networking of information processing stages that involve only biochemical processes, aiming at eliminating the “wires” and “batteries” and reducing the overall need for electrical power supplies at the stages of information processing, carried out on-site in implantable devices.

Described herein are techniques, systems and apparatus for implementing biochemical analysis and data processing based on biocomputing with enzyme based logic gates that eventually could be used for real-time field monitoring of injured people. For example, biocomputing systems described herein can analyze biochemical markers, characteristic of different pathophysiological conditions to illustrate the potential biomedical application of biocomputing. In addition, signal processing techniques are described to apply a new set of logic operations which allow better discrimination of different pathophysiological conditions. Experimental data described herein illustrate the ability to generate distinct patterns of output signals from different combinations of biochemical input signals, characteristic of various normal and abnormal physiological conditions. Biochemical logic gates can create high fidelity diagnosis for a greatly improved decision-making, leading to an optimal timely therapeutic intervention and to improved survival of injured soldiers and civilians. To provide these advantages in addition to providing a minimally invasive enzyme logic gate based sensing device, we stepped forward from optical analysis of the enzyme logic operations to electrochemical transduction of the output signals. Both signal transduction methods (optical and electrochemical) can be employed and compared, demonstrating good consistency of the results.

Logic Gate Processing

Different types of injury can result in distinct pathophysiological changes reflected by changes in the concentrations of many biochemical substances in a body. Some of these biomolecules undergo major concentration changes during a given injury and could be selected as biological signaling markers useful for biochemical processing, i.e., as input signals for enzyme logic gates/circuitries. In the example described in FIGS. 1A, 1B and 1C, glucose, lactate and norepinephrine (NE) are used as physiological markers that signal on different kinds of injuries to demonstrate the concept of the biochemical signal processing and autonomous decision-making. Examples of Specific patterns in the concentrations of these markers can provide sufficient information to identify the type of injury occurred in a body. Abnormal increase in the concentration of glucose might originate from hemorrhagic shock (HS), while higher than normal physiological concentration of lactate could be caused also by HS or/and trauma brain injury (TBI). A high concentration of NE can be indicative of any traumatic injury. Thus, glucose, lactate and NE can be applied as chemical input signals for the enzyme logic circuitry. The digitized signals were considered as logic 0 when the inputs were applied at their physiologically normal concentrations: 4 mM glucose, 2 mM lactate and 2.2 nM NE. Abnormal high concentrations of glucose (30 mM) characteristic of HS, of lactate (13 mM) observed in case of both TBI and HS, and of NE (3.5 μM) typical for any traumatic injury were considered as logic 1 values for the input signals. For illustrative purposes, only two concentrations of the chemical input signals were applied corresponding to the digital values of 0 and 1, while their intermediate concentrations were considered as digitally undefined.

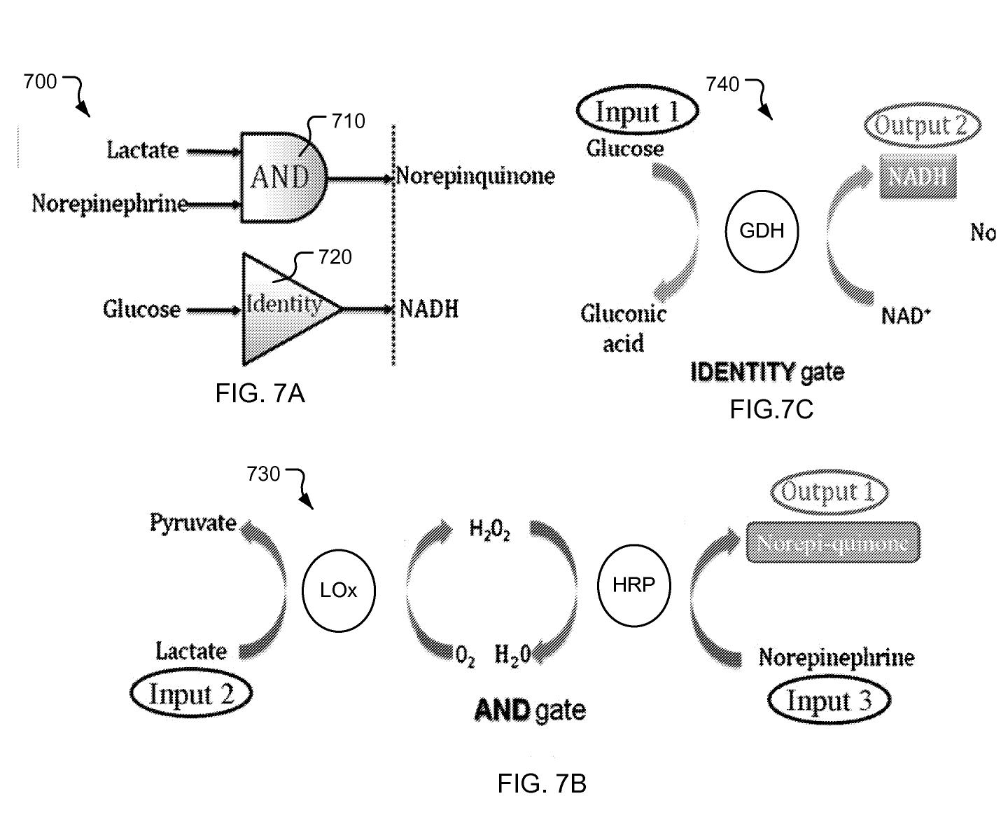

FIG. 7A is a block diagram showing an exemplary enzyme logic circuitry for analyzing three chemical input signals. The enzyme logic circuitry 700 analyzes three chemical input signals (glucose, lactate and NE) in two parallel-functioning biocatalytic pathways 730 and 740 using an AND logic gate 710 and an IDENTITY logic gate 720. The AND logic gate 700 can include LOx and HRP activated by the lactate and NE input signals and produced norepi-quinone (NQ) as an output signal. Lactate was oxidized by LOx in the presence of oxygen resulting in the biocatalytic formation of H₂O₂, which activated the oxidation of NE by HRP. The oxidation of NE yielding norepi-quinone (NQ) proceeded only in the presence of both input signals (lactate and NE), mimicking a Boolean AND logic operation, while the absence of any of these two inputs results in the inhibition of the biocatalytic chain reaction. The biocatalytic pathway 730 in FIG. 7B illustrate the operation of the AND gate 710. The IDENTITY logic gate 720 was based on GDH which translated the glucose input signal into an NADH output signal 730 as shown in FIG. 7C. The described enzyme logic system can provide simultaneous operation of all biocatalytic reactions in a single solution with the readout of only two output signals for three applied inputs logically processed by the enzyme system.

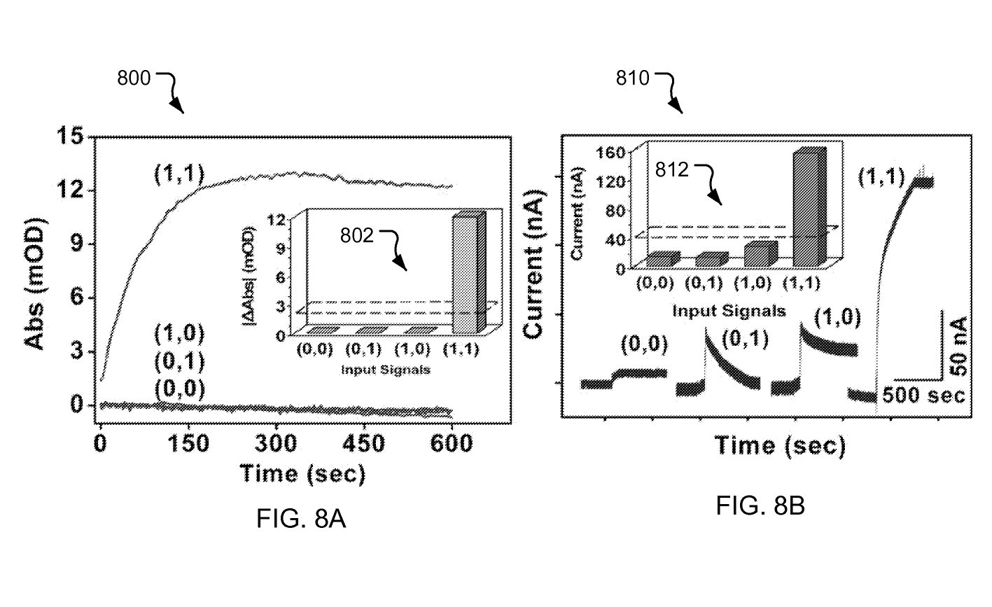

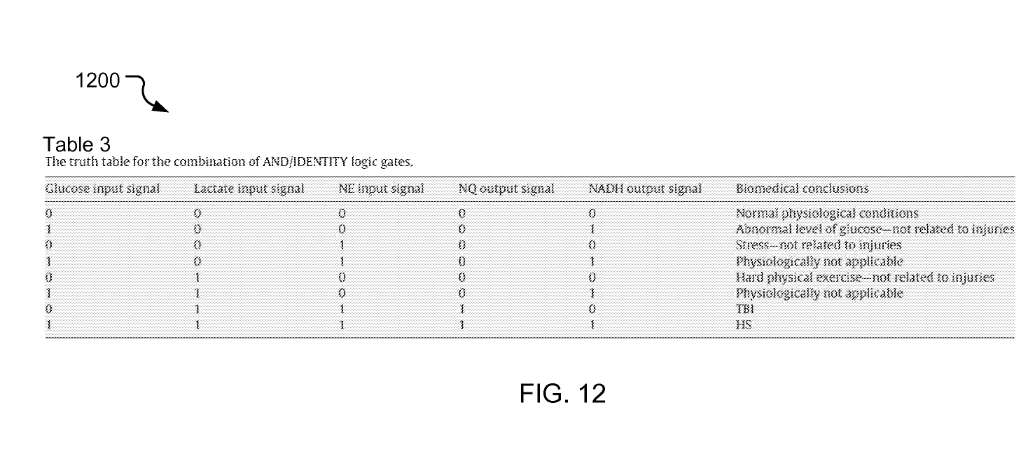

The performance of each logic gate can be analyzed separately. In addition, the performance of all logic gates 710 and 720 can be applied simultaneously. The formation of NQ upon the operation of the AND gate 710, was analyzed for four different combinations of the two input signals (lactate and NE): 0,0; 0,1; 1,0 and 1,1 being at their logic values of 0 and 1. The formation of NQ was detected by optical absorbance measurements at λ=465 nm (800) as shown in FIG. 8A, and by electrochemical current measurements at the potential of −0.25V (810) as shown in FIG. 8B. Such detection potential was selected based on square-wave voltammetric measurements (not shown). The built up of the output signal, corresponding to the formation of NQ, above the threshold values of 2 mOD are as shown in the inset 802 of FIG. 8A, and 40 nA as shown in the inset 812 of FIG. 12B for the optical and electrochemical measurements, respectively, were defined as the logic value 1 for the output signal of the AND gate. Otherwise, the output signal was defined as a logic 0 value. A 1,1 combination of the input signals resulted in the output signal 1 resembling the features of the AND logic gate and signaling about the formation of NQ above the threshold value. The obtained optical and electrochemical results show good consistency. NQ was also produced in a lower concentration (below the threshold value) for the 0,0, 0,1 and 1,0 combinations of the input signals, because the logic 0 values of the input signals were not physically zero, but corresponded to the normal physiological concentrations of lactate and NE.

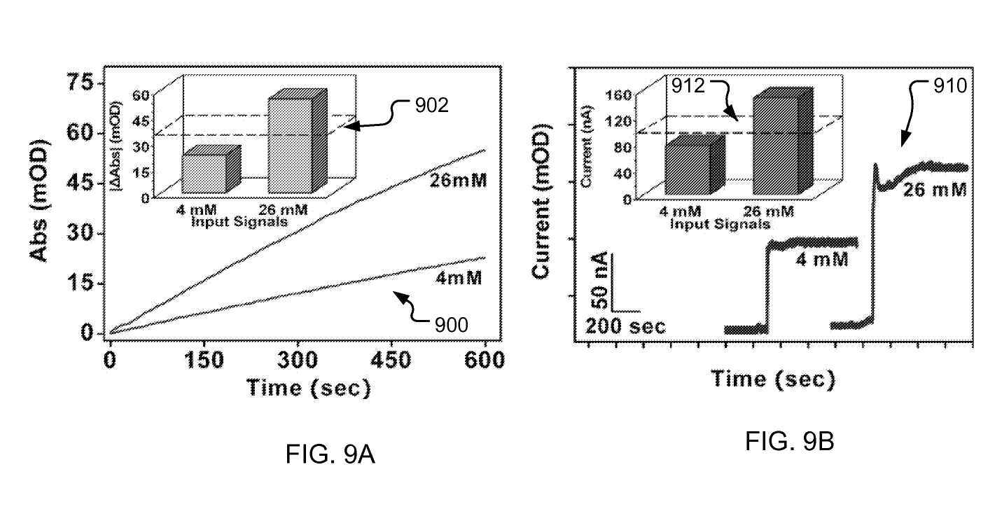

The second logic gate 720 in FIG. 7A can include a single enzyme (GDH) and can be adapted to discriminate between normal and pathological concentrations of glucose. The input of glucose was applied at two different levels of 4 mM and 26 mM being considered as logic 0 and 1 values, respectively as shown in FIGS. 9A and 9B. The biocatalytic formation of NADH as the result of the biocatalytic reaction was considered as the output signal. The production of NADH was monitored by optical absorbance measurements at λ=340 nm (900) as shown in FIG. 9A, and by amperometric analysis carried out at +0.75V (910) as shown in FIG. 9B. The exact potential for the oxidation of NADH was determined using square wave voltammetry of NADH (not shown). The NADH output signal above the threshold values of 38 mOD as shown in the inset 902 of FIG. 9A and 100 nA as shown in the inset 912 of FIG. 9B for the optical and electrochemical measurements, respectively, were defined as the logic value 1 for the output signal of the IDENTITY gate. Otherwise, the output signal was defined as logic 0 value. Both methods, optical and electrochemical, resulted in the consistent data demonstrating good agreement. The logic 0 value of the input signal was not physically zero, but rather corresponded to a physiologically normal glucose concentration, 4 mM, thus resulting in a relatively high level background signal.

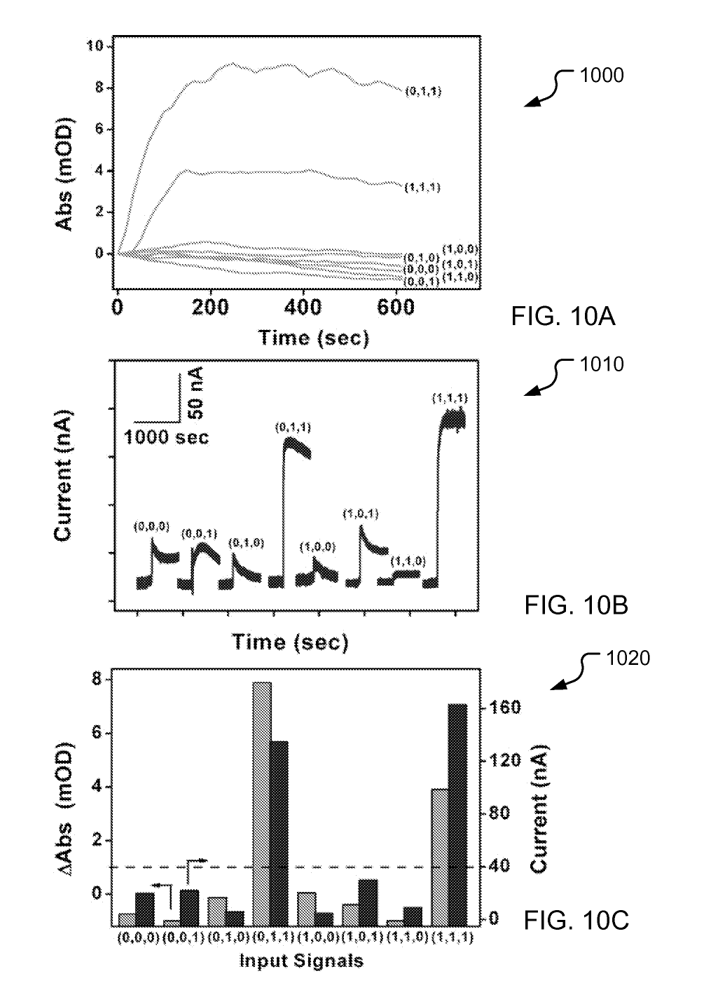

In addition, described is the simultaneous operation of both logic gates upon concerted operation of GDH, Lox and HRP activated by three input signals (glucose, lactate and NE) in connection to eight different input combinations. The definition of logic 0 and 1 values of the input signals was the same as described above for the individual logic gates, and the analysis of the output signals was performed by the optical and electrochemical means similarly to the procedures described above. FIGS. 10A and 10B show the optical (1000) and electrochemical (1010) detections for the output signal produced by the AND gate in the form of NQ. The high level signals corresponding to the NQ production were observed only for the 1,1 combination of the lactate and NE input signals, regardless of the logic value of the glucose input (in other words, input signal combinations of 0,1,1 and 1,1,1, where the first logic value is reserved for glucose, resulted in logic 1 output value of NQ). FIG. 10C summarizes the output optical and electrochemical signals 1020 for all combinations of the input signals. The threshold values of 1 mOD and 40 nA were applied to separate the logic 0 and 1 values of the NQ output signal.

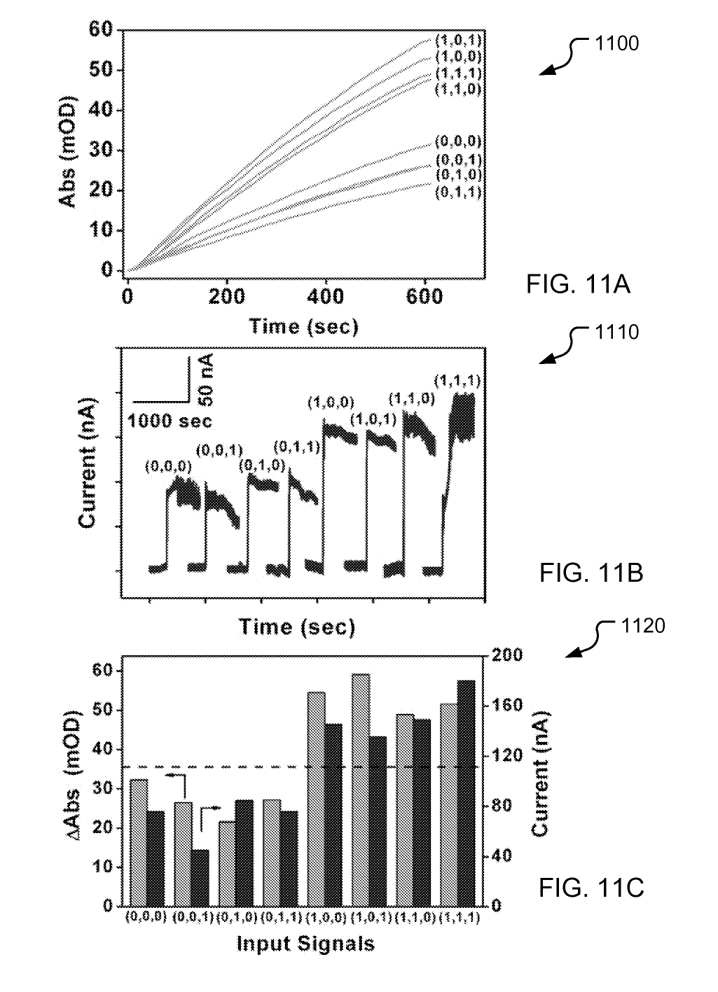

FIGS. 11A and 11B display the optical (1100) and electrochemical (1110) detection for the output signal produced by the IDENTITY gate in the form of NADH. The high level signals of the NADH production were observed only for the logic value 1 corresponding to the glucose input regardless of the logic values of the lactate and NE inputs (in other words, for input signal combinations of 1,0,0; 1,0,1; 1,1,0 and 1,1,1, where the first logic value is reserved for glucose, resulted in logic 1 output value of NADH). FIG. 11C summarizes the output signals 1120 obtained by both methods for all combinations of the input signals. The threshold values of 35 mOD and 110 nA were applied to separate logic 0 and 1 values of the NADH output signal. The logic 0 value of the glucose input signal corresponded to the normal physiological concentration of glucose, 4 mM, which is only 6.5-fold lower than the abnormal concentration defined as the logic value 1. This, along with the non-linear concentration dependence (associated with the enzyme kinetics) resulted in a relatively small difference between the physical values of the output signals corresponding to their logic 0 and 1 values. The resulting data demonstrate good agreement between the optical and electrochemical analysis of the logic circuitry operation. Also, the enzyme logic “machinery” operated in the same solution without any visible cross-reactivity.

The physiological conditions in the studied model system can be analyzed based on the comparison of the two output signals generated by the AND/IDENTITY gates as shown in Table 3 (1200) of FIG. 12. Different diagnoses could be derived from various combinations of the output signals. The output signal combination 0,0 can be obtained for three different combinations of the input signals: 0,0,0; 0,0,1 and 0,1,0. The first situation (0,0,0) corresponds to all three input signals being at their normal physiological levels, thus reflecting normal (i.e., non-injury/trauma) physiological conditions. The second input pattern (0,0,1) corresponds to an increased level of NE alone, thus reflecting a stress situation without any physical injuries. The third combination of the input signals (0,1,0) corresponds to the abnormally increased level of lactate, which might originate from physical exercises, still without any evidence of injury. Three scenarios do not signal about any injury and do not require any medical intervention. The output signal combination 0,1 could theoretically originate from three different patterns of the input signals: 1,0,0; 1,0,1 and 1,1,0. However, only the first of them is physiologically possible, while two others are applicable only in a model system in vitro. Indeed, the normal concentration of lactate is impossible when there are increased levels of NE and glucose, both being signals of an injury. Similarly, a physiologically normal NE level is impossible in the presence of high concentrations of lactate and glucose, which can occur only in the case of injury. The only physiologically meaningful combination of the input signals (1,0,0) corresponds to a high level of glucose, which is not related to injuries (but rather to a diabetes condition). Therefore, the output signal combination 0,1 can be an unlikely situation, which is of little or no relevance to injury. The most valuable output of the gate system corresponds to the last combinations of the output signals: 1,0 and 1,1, which originate from the input signal combinations 0,1,1 and 1,1,1, respectively. Both of these combinations of the biomarker input indicate serious injuries. The first output signal pattern (1,0) corresponds to lactate and NE being at high concentrations reflecting a physiological situation characteristic of TBI; the second pattern (1,1) corresponds to all three markers (glucose, lactate and NE) being at high concentrations, thus indicating a likely occurrence of HS. These two combinations of output signals allow discrimination between two different forms of severe injury originating from TBI and HS and require an immediate medical intervention.

Chemicals and Reagents

Lactate oxidase (LOx) (E.C. 1.1.3.2) was purchased from Genzyme Corp. and was purified as follows. About 100 μL of Lox (0.5 units μL⁻¹) in a 50 mM phosphate buffer solution (pH 7.4) was taken in 100 kDa Centrisart ultra-filtration tube and centrifuged at 7000 rpm for 15 min at 4° C. The sediment was washed with 80 μL of phosphate buffer and centrifuged. The process was repeated five times. All other chemicals and enzymes were purchased from Sigma-Aldrich and used as supplied without any further purification: β-nicotinamide adenine dinucleotide (NAD⁺), D-(+)-glucose, (L)-norepinephrine (NE), L-(+)-lactic acid. Other enzymes used were horseradish peroxidase (HRP) type VI (E.C. 1.11.1.7) and glucose dehydrogenase (GDH) from Pseudomonas sp. (E.C. 1.1.1.47). Ultrapure water (18.2 MΩm) from NANOpure Diamond (Barnstead) source was used in all of the experiments.

Instruments

The optical measurements were performed using a Shimadzu UV-2450PC spectrophotometer. The electrochemical measurements were performed using a CH Instrument Model CHI630C with an electrochemical cell consisting of a glassy carbon working electrode, Ag/AgCl (3M NaCl) reference and a platinum wire counter electrode.

Logic Gates and Input Signals

An exemplary AND logic gate implemented in this aspect can include (or consist of) a 1 mL of phosphate buffer solution, 50 mM, pH 7.4, containing both LOx, 0.1 units mL⁻¹ and HRP, 5 units mL⁻¹. The exemplary biochemical input signals were NE and lactate. The exemplary input concentrations of NE were 2.2 nM and 3.5 μM selected as digital 0 and 1 signals, respectively. Lactate concentrations of 2 mM and 13 mM were defined as exemplary digital logic inputs of 0 and 1, respectively. Norepi-quinone (NQ) biocatalytically produced in the AND logic gate was defined as the exemplary output signal and its production was monitored by the optical absorbance measurements at λ=465 nm and by electrochemical current measurements at E=−0.25 V. The exemplary IDENTITY gate included (or consisted of) a 1 mL of phosphate buffer solution, 50 mM, pH 7.4, containing GDH, 0.025 units mL⁻¹ and 20 μMNAD⁺. This gate used only one input signal of glucose. The glucose concentrations of 4 mM and 26 mM were defined as digital logic inputs of 0 and 1, respectively. The output signal of the IDENTITY gate was defined as a concentration of the biocatalytically produced NADH, which was measured optically at λ=340 nm and electrochemically at E=0.75 V. An exemplary combined biochemical system made up of the AND and IDENTITY gates can include all enzymes/cofactors operating as the biocomputing “machinery” (LOx, HRP, GDH, and NAD⁺). In addition, the biochemical system can use three chemical inputs, including glucose, lactate and NE, applied in the concentrations corresponding to their digital 0 and 1 values. The optical output signals corresponding to the formation of NQ and NADH in the same solution were measured at 465 nm and 340 nm, respectively. In the case of electrochemical measurement, the output signal of NQ and NADH were measured at −0.25V and +0.75 V, respectively. All measurements were carried out at ambient temperature (23±2° C.).

The described approach to multi-signal biosensing can allow qualitative evaluation of the biochemical information in terms of YES-NO providing the background for logic analysis of complex patterns of biochemical signals. The quantitative assessment of the biochemical information can be provided by the threshold values between 0 and 1 logic values. The described in vitro model system demonstrated the applicability of enzyme logic gates to the assessment of biochemical signals and processing their combinations at physiological concentrations relevant to normal and pathophysiological conditions associated with TBI and HS. The described systems demonstrated stable information processing and generated distinguishable patterns of the output signals from different combinations of biochemical input signals, characteristic of various normal and abnormal physiological conditions. The developed approach can be implemented to novel digital biosensors processing multiple-biochemical input signals and producing a combination of output signals dependent on the whole pattern of various input signals. The biochemical signals are processed by chemical means based on the enzyme logic system and the difference between different physiological scenarios can be directly derived from the chemically processed information, hence obviating the need for computer analysis of the biosensing information. The biochemical signals were transduced by optical and electrochemical means to demonstrate the broad applicability of the developed approach. For practical applications, particularly related to the battlefield analysis of physiological conditions of injured soldiers, we plan an electrochemical approach based on the application of minimally invasive on-body electrodes. In addition to the analysis of the data, the output signals might be directed to chemical actuators (e.g. signal-responsive membranes) leading to an on-demand drug release. We anticipate that such biochemical logic gates can facilitate decision-making in connection to an autonomous feedback-loop drug-delivery system and can revolutionize the monitoring and treatment of injured soldiers and civilians and enhance their survival rate.

Enzyme-Logic Biosensor for Point-of-Care Diagnostics of Cardiac Events

Acute myocardial infarction (AMI) is a life-and-death emergency, where every second counts. While AMI victims can benefit from new medications and treatments, to be effective these drugs should be administrated rapidly after the first heart attack symptoms appear. Rapid and reliable diagnosis of cardiac abnormalities can allow for immediate medical intervention, leading to decreased mortality. Accurate diagnosis can also reduce unnecessary hospital admissions or discharges and reduce related health care costs.

Cardiovascular disorders display a wide range of characteristics and hence provide a challenging domain for diagnosis. Particular attention has been given to the development of blood tests for detecting injury to the heart muscle (myocardium) as early as possible among people with chest discomfort or other indicators of a potentially serious heart condition. Such blood tests for cardiac markers can assist physicians in their assessment and diagnosis of acute coronary syndromes and aid them in identifying and managing high-risk patients. Creatine kinase-MB (CK-MB), troponin T (cTnT), troponin I (cTnI), myoglobin, homocysteine and C-reactive protein (CRP) are all used for the assessment of the suspected acute myocardial infarction. However, markers such as the troponins, which persist in blood for a long period of time after an initial injury, may be of no value in detecting reinfarction. Such testing of individual cardiac markers commonly relies on time-consuming immunoassays. The biochemical diagnosis of AMI can be confirmed by observing a serial rise and fall in the serum activity of creatine kinase (CK) and its MB isoenzyme (CK-MB). Although the use of these enzyme markers enjoys widespread acceptance, both CK and CK-MB assays, have deficiencies. Serum CK-MB is considerably more specific for myocardial damage than is serum total CK, which may be elevated in many conditions where skeletal muscle is damaged. Consequently, CK should not be used for the diagnosis of myocardial injury unless used in combination with other more specific cardiac markers.

Between 70% and 80% of patients with an acute myocardial infarct have ischemic chest pain at presentation, but only 25% of those admitted with chest pain can have an infarct confirmed. Unstable angina may be the diagnosis in a further 30%, which means that a significant number of patients are unnecessarily admitted. Conversely 5% may be sent home inappropriately because of the diagnostic limitations of the ECG and creatine kinase-MB (CK-MB) measurements. Thus, there may be no need for laboratory confirmation of the diagnosis if the ECG identifies acute myocardial infarction but the initial ECG is only abnormal in 50% of cases. In summary, the NACB (National Academy of Clinical Biochemistry) Committee recognizes the limitations of myoglobin and CK-MB isoforms and encourages continued research into earlier markers, particularly if they are more specific for myocardial necrosis. Even if they were the perfect assay, they need to be performed in hospital facilities requiring blood sample and analysis, which can take the most critical hours to get the results.

In one aspect, the described techniques, systems and apparatus can be used to implement an enzyme logic system to provide immediate diagnostic information on multiple conditions at the point-of-care and provide a reliable point-of-care diagnosis of life-threatening heart problems. Cardiovascular disease is the number one leading cause of death and immediate and reliable diagnostic information regarding unpredicted cardiovascular events can be crucial for identifying patients experiencing such events, and hence for providing a timely life-saving medical intervention. Identifying patients with myocardial injury remains a major challenge in emergency medicine.

The described techniques, materials, apparatus and systems can be used to design, optimize, characterize and test an enzyme-logic bioelectronic sensor that provides a Boolean logic-based assessment of suspected acute cardiac events, independent of chest discomfort or other common warning signs. By employing multiple key cardiac biomarkers as inputs for the enzyme gates, monitoring patterns in the concentration changes of such multiple markers, and processing the signal information biochemically, the described ‘Cardio-Logic’ sensor system can provide high-fidelity point-of-care diagnostics of life-threatening heart problems compared to single or parallel biomarker sensors. Such reliable and rapid logic-based diagnosis can facilitate the rapid ‘on-the-spot’ identification of high-risk patients and a timely medical intervention, and can thereby lead to increased survival rates. Integration of biocomputing elements with sensing processes may rely on multiple inputs (e.g., markers) to trigger a cascade of enzymatic reactions, along with by biochemical processing, to give a final answer on the patient physiological condition in a digital (“YES” or “NO”) format. The described techniques, materials, apparatus and systems can include both single-use sensing devices and on-body continuous monitoring of high-risk patients.

A described sensing strategy based on biochemical logic systems can be implemented to provide reliable point-of-care diagnosis of life-threatening heart conditions. For example, the described techniques can be used to design, optimize and test an enzyme-logic based bioelectronic sensor system for ‘on-the-spot’ monitoring of multiple cardiac markers, thereby providing rapid and reliable detection of acute cardiac events, independent of chest discomfort or other common warning signs.

Enzyme-Logic Assessment of Cardiac Events:

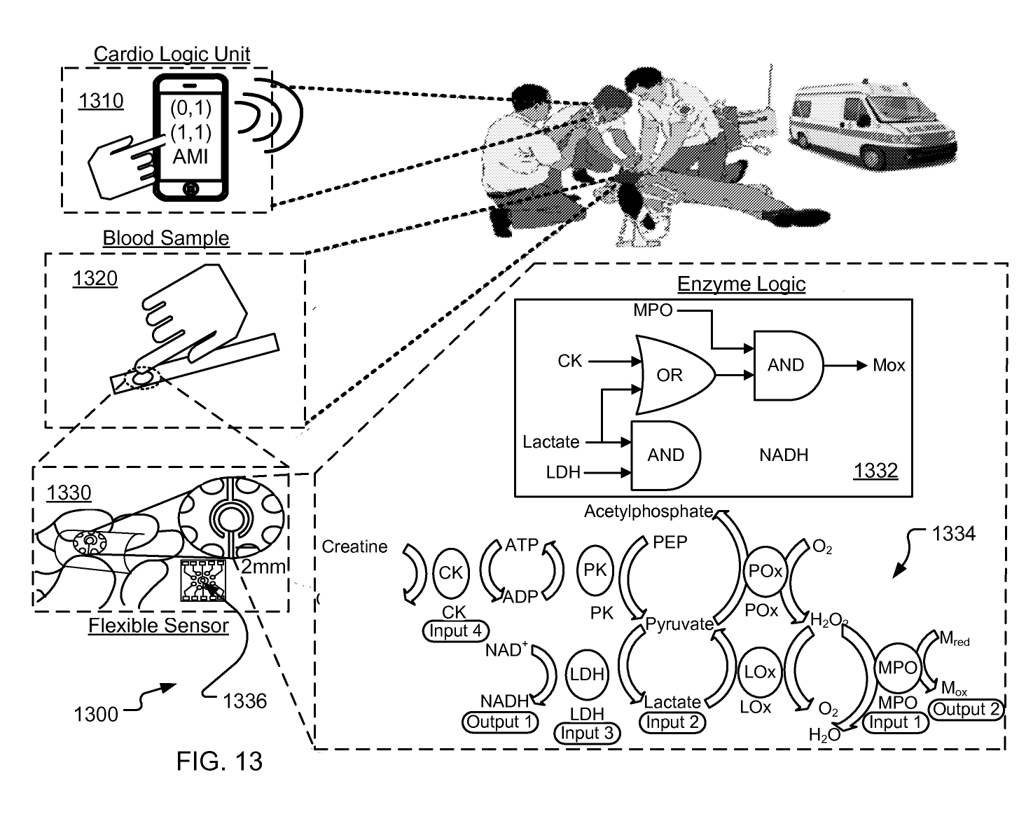

FIG. 13 shows an exemplary enzyme-logic based cardiac sensor system 1300 for reliable and rapid point-of-care diagnostics of cardiac events. A cardiac logic unit 1310 can interface with a flexible sensor 1330 to analyze a blood sample 1320. The flexible sensor 1310 can include printed electrode circuitry 1336 and an enzyme logic 1332 that operates in a biocatalytic cascade 1334 for performing logic processing of four biochemical signals: Myeloperoxidase (MPO), lactate dehydrogenase (LDH), creatine kinase (CK), and generating two output signals: NADH and oxidized redox mediator (Mox). These outputs yield four possible symptoms to be detected on the cardio logic unit 1310, which can be designated as a healthy heart (0,0), unhealthy body tissue (1,0), a heart related illness different from Acute myocardial infarction (AMI) (0,1) and AMI (1,1).

An exemplary cardio-logic system can include several concatenated logic gates operating in a concerted manner that are capable of processing the biochemical information received in the form of various combinations of chemical signals according to the principles of Boolean logic. Integration of biocomputing elements with sensing processes can allow multi-signal analysis followed by biochemical processing of the data, giving the final answer regarding the physiological conditions of a patient in a digital (“YES” or “NO”) format. The high dynamic range in the concentration of products associated with enzymatic processes closely mimics the high noise margins associated with digital electronic logic gate technology. In this manner, a physiologically relevant threshold corresponding to a specific concentration of input markers can be implemented electronically, above which a positive diagnosis would result.

Results show the ability of such logic-gate biocomputing systems to distinguish physiological conditions relevant to different battlefield injuries, including a traumatic brain injury and hemorrhagic shock. Similarly, the described biochemical logic systems can offer considerable promise for assessing the states of multiple cardiac biomarkers, leading to high-fidelity diagnosis of unpredicted cardiovascular events. Such enzyme-based information processing, mimicking a Boolean digital logic, can offer great promise for reliable clinical diagnostics and improved decision making, while reducing the overall power needs at the stage of information processing. The proposed enzyme-logic bioelectronic sensor can provide high-fidelity diagnosis of cardiac events (compared to current single or parallel biomarker sensors), leading to ‘on-the-spot’ identification of high-risk cardiac patients and to an enhanced survival rate. Such logic-gate biocomputing sensor system can successfully address the major challenge of reliable point-of-care testing of cardiovascular disorders. In addition to the development of enzyme-logic based cardiac diagnostics, the new biocomputing approach can eventually lead to continuous monitoring of different diseases in connection to a ‘Sense/Alert/Treat’ approach, thereby providing an autonomous on-demand therapeutic intervention.

Enzyme-Logic Bioelectronic Sensor System

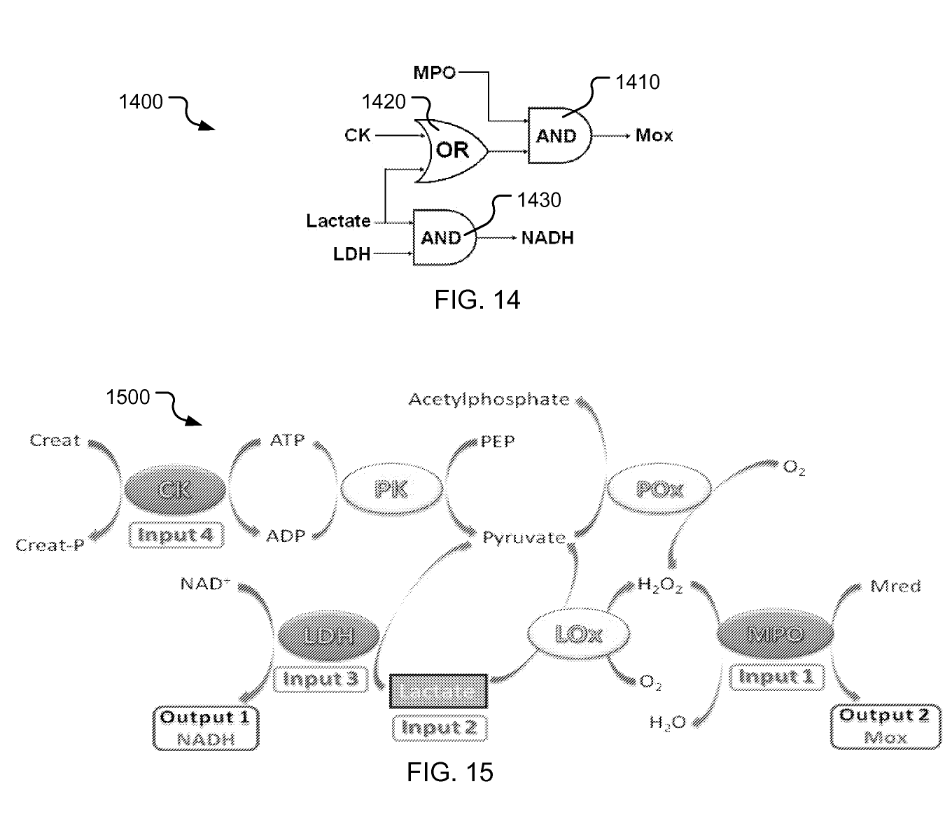

FIG. 14 shows an exemplary circuit diagram of a cardio-logic system 1400. The cardio-logic system can include several concatenated logic gates 1410, 1420 and 1430 operating in a concerted manner that are capable of processing the biochemical information received in the form of various combinations of chemical signals according to the principles of Boolean logic. For example, the enzyme-based logic gates can include two AND gates 1410 and 1430 and one OR gate 1420 performing specific biocatalytic reactions towards reliable diagnosis of cardiac events. The cardio-logic system 1400 can be implemented to be substantially similar to the enzyme logic 1332 of FIG. 13. FIG. 15 is a diagram showing an exemplary biocomputing logic network 1500 with built-in logic operations performed by the concatenated enzyme-based gates of the biochemical pathway 1334 of FIG. 13. The logic network can include four biomarkers as input signals relevant to the diagnosis of AMI and other cardiac events.

Described are experimental design and theoretical modeling and optimization of enzyme-based logic gates and their concatenated networks connected with a bioelectronic sensor transducing the biochemical signals to an electronic output. The multi-enzyme networks can process the biochemical information in the form of chemical signals represented by different physiological markers. The output signals generated by the biocomputing systems can be transduced electronically, leading to a unique multi-signal biosensor where the information is processed by biochemical means.

Techniques can include the design of biochemical logic networks composed of enzyme-based logic gates and their concomitant integration with electronic devices. The multi-reagent architecture on the electrochemical transducer can be used towards an efficient enzyme-logic gate operation and small dimensions for single-use testing. Thick-film sensor strips (e.g., flexible sensor 1330), containing the dry reagent layer and screen-printed electrodes (e.g., printed electrode circuitry 1336) can be used to facilitate such enzyme-logic based point-of-care cardiac diagnostics in connection with fingerstick (microliter) blood samples.

The flexible sensor strip can include the printed reference and counter electrodes (Ag/AgCl and carbon, respectively), along with two working electrodes for the simultaneous monitoring of the NADH and oxidized redox mediator (Mox) outputs. The carbon working electrodes can be modified (by incorporating appropriate catalysts to the ink) to ensure favorable amperometric detection of NADH and Mox. The ‘loading’ level and printing conditions can be tailored to meet the requirements of favorable signal-to-background characteristics. The minimization of surface fouling (common to NADH detection) can also be considered when selecting the catalyst (although this is not a major concern in single-use sensors).

The composition of the ‘dry reagent layer’, containing various enzyme and co-substrate components of the logic network machinery is described along with the corresponding buffer salts (to generate a fixed pH). Two routes can be used for generating the reagent layer and ensuring its rapid dissolution and dispersion. One scheme can include a porous reagent-containing disk, prepared by soaking a filter paper disk in the reagent mixture and drying in vacuum. The second scheme can be based on depositing the reagents onto the top plate of a capillary-fill (thin-layer) sensor device. Systematic optimization can allow tuning of the levels of LOx, PDX, PK, ATP, NAD⁺, M and PEP in the reagent layer to ensure a proper reaction stoichiometry (so that the output signals correlate with the input levels), ‘masking’ the effect of fluctuations in the blood level of these constituents, and rapid dissolution of its constituents upon exposure to the blood sample. The gap (between the 2 plates), controlling the sample volume, and the electrode dimensions and geometry, can also be optimized.

The operating conditions of the enzyme-logic bioelectronic sensor can be assessed and optimized. These can include the detection potentials and reaction/delay times. The resulting enzyme-based logic sensor strip can be critically tested in the presence of relevant concentrations of cardiac biomarkers (inputs), reflecting different clinical scenarios and the dynamics of cardiac physiology. The multiple physiological signals can be logically processed in Boolean fashion by the biochemical logic system and then the output signal can be electronically transduced. Following these testing, the sensor can be validated using relevant clinical samples following the conventional in vivo experiments ladder. The storage stability of the logic-based biosensor can also be evaluated.

The new logic-gate biocomputing sensor system can successfully address the major challenge of reliable point-of-care testing of cardiovascular disorders. Such use of enzyme-logic based diagnosis is unique as it relies on patterns of multiple markers and biochemical processing of the signal information for reliably identifying cardiac abnormalities. It can thus result with a rapid high-fidelity point-of-care diagnosis of acute cardiac events, thereby leading to greatly increased survival rates. While the concept of cardio-logic based diagnostics is presented here in connection to single-use point-of-care testing, the described techniques, materials, apparatus and systems can eventually lead to autonomous continuous monitoring of high-risk cardiac patients in connection to a ‘Sense/Alert/Treat’ approach. Application of biocatalytic processes for logic-gate sensing operations can introduce a new diagnostic paradigm.

The Selected Biomarkers Appear in the Earliest Stages of AMI:

Creatine kinase (CK) and myeloperoxidase (MPO) are biomarkers that appear in the earliest stages of AMI. Other non-specific markers include lactate dehydrogenase (LDH) and lactate. The reliability of the output can be increased by adding redundancy to the enzyme logic system. For example, a blood level increase of CK indicates either skeletal or a myocardial muscle damage. The enzymatic activity of CK, as routinely determined in emergency room patients, shows a specificity report of serum CK-MB for the detection of AMI very close to 100%, while the total CK only reaches up to 70%. The increase in LDH level after myocardial infarction is determined via the LD₁ isoenzyme. Other LD isoenzymes can be found in different tissues (lungs, kidneys, brain or liver). LDH is a non-specific marker. Myeloperoxidase (MPO) is a hemo-containing peroxidase enzyme presents mainly in polymorphonuclear neutrophil granulocytes. Infiltration by these leukocytes is observed in the damaged atherosclerotic plaques associated with acute coronary syndromes. Serum MPO has been shown as a cardiovascular marker by patients with chest pain. The blood level of these markers under normal physiological conditions, 10-40 mU·mL⁻¹, is low enough to avoid possible false positive signals. However, upon a pathological situation, the concentrations of these markers increase up to 100-fold.

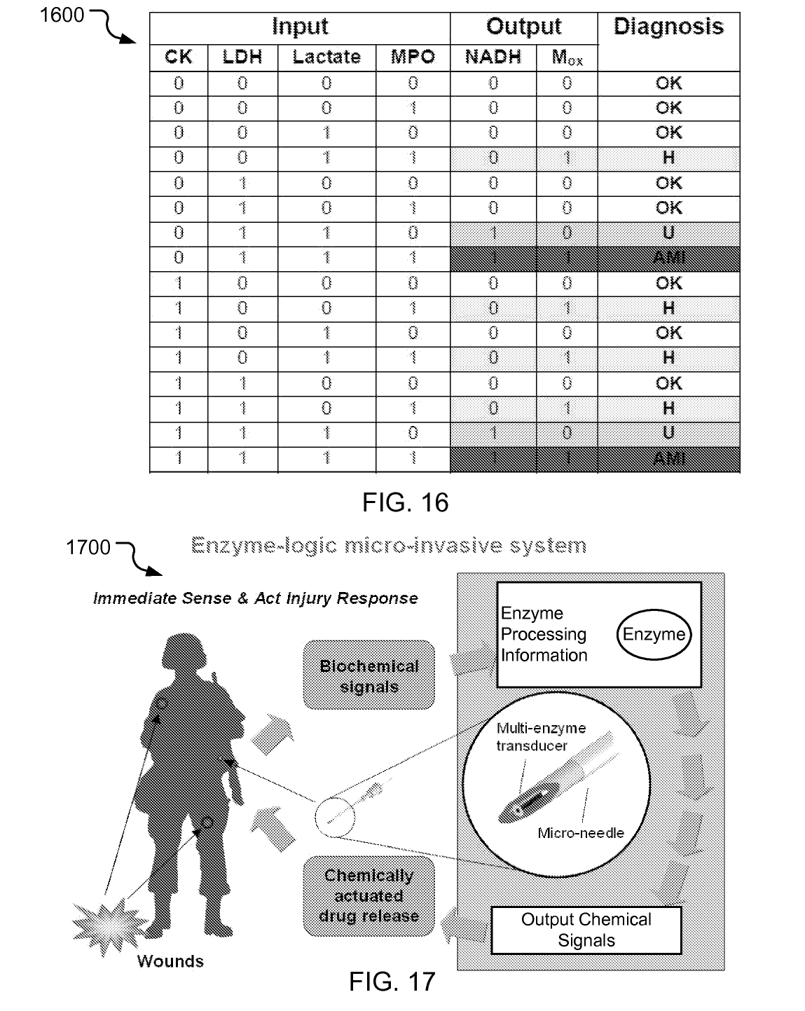

The logic analysis of the different input combinations requires additional components, which can comprise the logic network machinery. This machinery includes three enzymes, pyruvate kinase (PK), lactate oxidase (LOx) and pyruvate dehydrogenase (PDH), and several chemicals such as ATP, NAD⁺, phosphoenolpyruvate (PEP) and oxygen along with a reduced output redox mediator (M), e.g., TMB or APTS (examples only for in vitro environment). The system is designed to provide two different output signals: production of NADH and oxidation of the redox mediator (M). Table 4 of FIG. 16 shows a truth table for the logic processing of the biochemical signals of FIG. 14 (OK—healthy heart; U—unhealthy body tissue; H—heart-related condition different from AMI; AMI—acute myocardial infarction).

These outputs yield four possible symptoms to be detected, which can be defined as a healthy heart (0,0), unhealthy body tissue (1,0), a heart-related illness different from AMI (0,1) and AMI (1,1), all labeled with different colors in the Truth Table 4. Eight input combinations give a (0,0) output, indicating that the person is healthy, or that there is not enough information (only one or two inputs present) collected to give a diagnosis. The output (1,0) is given by the presence of lactate and LDH, which indicates a non-defined soft-tissue related health problem. The output (0,1), MPO, is indicative of a heart problem; however, the addition of one or two other non-specific markers (lactate and/or CK) can indicate AMI. A (1,1) output is a strong signal of AMI in progress and occurs only in the presence of elevated levels of at least three input markers, MPO, lactate and LDH. Our initial effort can critically evaluate the new biocatalytic logic gates in solutions (using soluble components of the network) for their response to the corresponding cardiac marker inputs and for potential ‘cross talks’ and interferences. Other concatenated enzyme-based logic gates, involving different marker inputs and biocatalytic cascades, can be designed and tested during the course of this activity.

The biochemical system outlined above is given only as an example of the practical realization of the invented device/process and it cannot limit the possible variants of the enzyme-logic systems. The disclosure thus covers a wide range of biochemical inputs, different transduction modes, and can be extended to the assessment of different diseases in connection to autonomous decision making (e.g., timely therapeutic intervention). The concept can be applied for both continuous monitoring of high-risk patients or for single measurements of patients experiencing cardiac events.

Described techniques can be used to design, optimize and critically test a multi-biomarker sensor system that includes concatenated enzyme-based logic gates for the reliable and rapid point-of-care identification of life-threatening heart-related problems among patients with chest discomfort. The enzyme-logic sensing system, interfaced with electronic transducers, can perform Boolean logic operations for high-integrity processing of the physiological information in order to provide a logic-based assessment of the patient's physiological condition during unpredicted cardiovascular events. By using multiple key cardiac biomarkers as inputs for the enzyme gates, the new biochemical logic sensor system would provide high-fidelity diagnostics of cardiovascular events, compared with common single biomarker sensors. Thick-film (screen-printed) electrochemical transducers, containing the dry enzyme-logic reagent layer, can thus be designed and optimized for facilitating such point-of-care cardiac diagnosis on undiluted microliter blood samples.

The described techniques, apparatus and system can be used to provide a reliable point-of-care diagnosis of life-threatening heart problems, and identify patients with myocardial injury. For example, the described techniques can be used to design, optimize, characterize and test an enzyme-logic bioelectronic sensor for providing a logic-based assessment of suspected heart problems, independent of chest discomfort or other common warning signs. By employing multiple key cardiac biomarkers as inputs for the enzyme gates, monitoring patterns in the concentration changes of such multiple markers, and processing the signal information biochemically, the described ‘Cardiac-Logic’ sensor system can be used to provide high-fidelity point-of-care diagnostics of life-threatening heart problems compared to single or parallel biomarker sensors. Reliable and rapid logic-based diagnosis can facilitate the rapid ‘on-the-spot’ identification of high-risk patients and a timely medical intervention, and can lead to increased survival rates.

The described techniques can include: (1) design and optimization of enzyme-logic gates performing Boolean logic operations in the presence of major cardiac biomarker inputs; (2) design and optimization of the miniaturized electronic transducer strip and of the dry reagent layer containing components of the logic network machinery; and (3) critical testing and validation of the enzyme-logic bioelectronic sensor system using physiologically relevant scenarios.

Enzyme Logic Gates for Assessing Physiological Conditions During an Injury: Towards “Smart” Digital Sensors and Actuators

In another aspect, described are biocomputing logic systems based on concerted operation of several enzymes designed to process biochemical information related to pathophysiological conditions originating from various injuries. Different biochemical markers were applied as input signals to activate the enzyme logic system. Physiologically normal concentrations of the markers were selected as logic 0 values of the input signals, while their abnormally increased concentrations characteristic of various injuries conditions were defined as logic 1 input values. Biochemical processing of different patterns of the biochemical markers resulted in the formation of output signals. Optical and electrochemical means were used to follow the output signals. The enzymatically processed biochemical information allowed distinguishing the difference between normal and pathophysiological conditions corresponding to trauma brain injury and hemorrhagic shock. The developed system is the first example of a biocomputing logic system applied for the analysis of biomedical conditions related to various injuries. We anticipate that such biochemical logic gates can facilitate decision-making in connection to integrated autonomous feedback system and hence to revolutionize the monitoring and treatment of injured soldiers and civilians. FIG. 17 shows a general concept of the “Sense & Treat” approach (1700) based on enzyme-logic gates processing of the biochemical information regarding the nature and severity of injury and initiating automated treatment by activating a drug-releasing chemical system.

Bio-Logic Analysis of Injury Biomarker Patterns in Human Serum Samples

In another aspect, digital biosensor systems can be implemented to analyze biomarkers characteristic of liver injury (LI), soft tissue injury (STI) and abdominal trauma (ABT). The digital biosensor systems can be optimized for their performance in serum solutions spiked with injury biomarkers in order to mimic real medical samples. The digital biosensor systems can produce ‘Alert’-type optical output signals in the form of “YES-NO” separated by a threshold value. The described techniques can provide reliable detection of injury biomarkers for making autonomous decisions towards timely therapeutic interventions, particularly in conditions when a hospital treatment is not possible. The enzyme-catalyzed reactions performing Boolean AND/NAND logic operations in the presence of different combinations of the injury biomarkers can allow high-fidelity biosensing. Robustness of the systems was confirmed by their operation in serum solutions, representing the first example of chemically performed logic analysis of biological fluids and a step closer towards practical biomedical applications of enzyme-logic bioassays.

Chemical and Biochemical systems switchable between two distinct states can be implemented in the formulation of molecular assemblies performing Boolean logic operations (e.g. AND, OR, NAND, NOR, XOR, etc.) and their networks mimicking digital computing processes. These systems can be applied as potential multi-signal analyzing biosensors with built-in Boolean logic resolved by chemical means without involvement of electronic computers. Analysis of various combinations of biochemical signals through biomolecular-implemented logic is particularly promising in biomedical applications. The digital nature of the output signals generated in the form of YES-NO separated by a threshold can allow alert-kind high-fidelity biosensing along with a proper therapeutic intervention. The use of enzymes as logic gates performing sensing that harnesses a Boolean logic architecture is a novel concept. This approach is extremely attractive since enzymatic systems can accept multiple biochemical signals and upon biochemical processing of the input information they can generate an output signal to activate an electrochemical transducer and/or a chemical actuator (i.e. drug-releasing membrane). The biomolecular logic analysis of multiple biomarkers appearing in complex combinations can call for concerted operation of several biocatalytic steps, while each one may require different conditions.

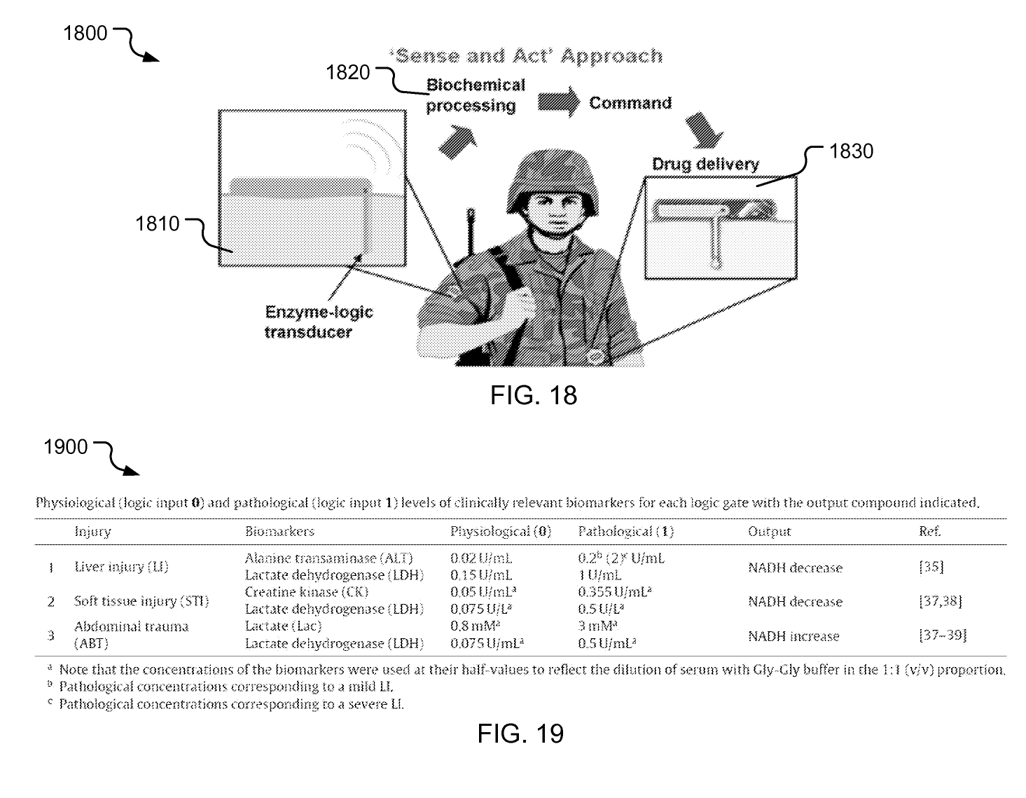

The described biochemical logic systems can use enzyme-based logic gates to provide reliable detection of injury biomarkers and for making autonomous decisions towards a timely therapeutic intervention during trauma or shock events, particularly in the case when a hospital treatment is not immediately possible, for example for injured soldiers on a battlefield. The described techniques, apparatus and systems can be applied in various implementations to provide a biochemical logic-based assessment of the overall physiological condition of a soldier during injury. The biochemical logic network systems can include enzyme-based logic gates performing specific biocatalytic reactions for the reliable diagnosis of an injury and eventually will enable the automated treatment of injured soldiers. Such systems will be activated by different biochemical input signals corresponding to various injury scenarios (brain injury, trauma, shock, fatigue, stress, etc.). Parallel activation of different gates would lead to distinct logic operations, reflecting the nature and severity of the injury, hence providing a reliable diagnosis essential for correct decision-making and automated therapeutic intervention according to a “Sense-and-Act” system 1800 as shown in FIG. 18. In the example shown in FIG. 18, a soldier is wearing an enzyme-logic transducer 1810 that perform biochemical processing 1820. Results of the biochemical processing can be used to provide commands to a drug delivery system 1830 to administer a corresponding drug at a corresponding dosage. The sense and act system 1800 can be applied to the analysis of biomarkers characteristic of various injuries using model buffer solutions. Among pervasive battlefield injuries, acute liver injury (LI), soft tissue injury (STI) and abdominal trauma (ABT) are the most common sustained by soldiers in combat. Described herein are various applications of digital biosensors with the built-in logic for the analysis of the LI, STI and ABT biomarkers in serum solutions mimicking the operation of future in-vitro and in-vivo multi-parameter biosensors.

Liver Injury (LI)

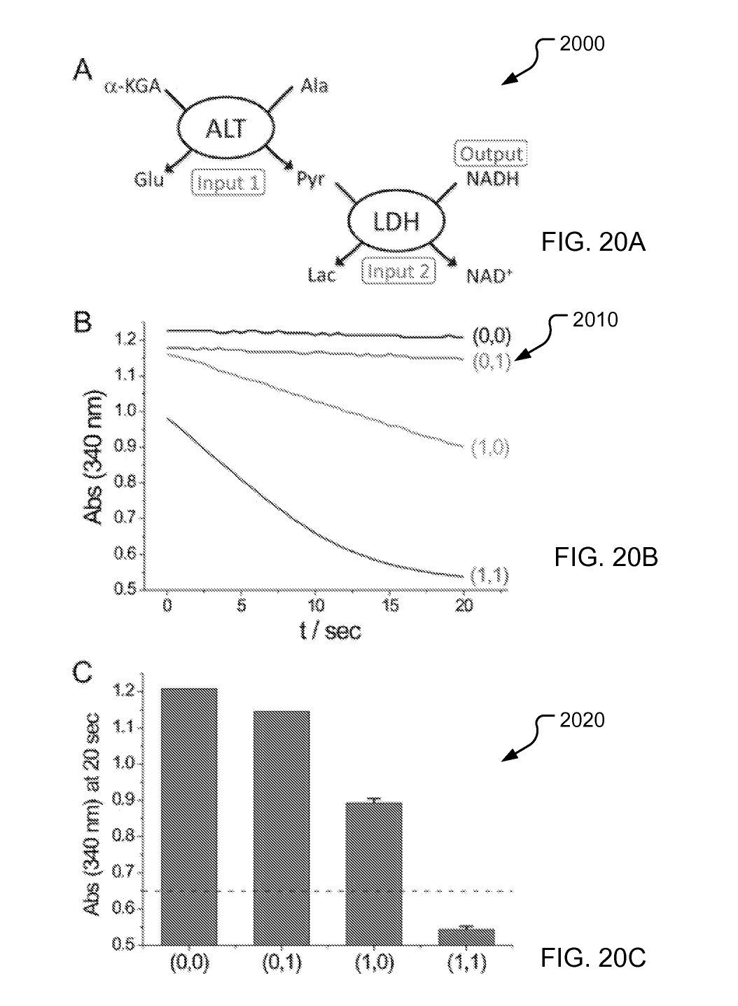

Two enzymes, ALT and LDH, were applied as exemplary biomarkers characteristic of liver injury. Their simultaneous increase in concentration, from normal to pathological levels (see Table 5 (1900) in FIG. 19), provides an evidence of LI conditions. The biochemical cascade catalyzed in the presence of the both enzyme-biomarkers can result in the oxidation of NADH to NAD⁺ (2000) as shown in FIG. 20A. The corresponding absorbance decrease (2010) is shown in FIG. 20B. The logic value of the output signal changes from the high ‘1’ value to the low ‘0’ value only upon the cooperative work of the both enzymes (logic inputs combination ‘1,1’), thus mimicking NAND (Not-AND) logic operation. It should be noted that the physical value of the output signal (decrease of the NADH absorbance) is also affected by other combinations of the input signals (‘0,0’; ‘0,1’; ‘1,0’) reflecting their non-zero physical values corresponding to the logic level ‘0’. A threshold at the absorbance of 0.65 measured at the sampling time of 20 sec allows clear separation of the logic ‘0’ and ‘1’ output values measured below and above the threshold (2020), respectively as shown in FIG. 20C. Thus, the optical output signal measured below an absorbance of 0.65 allows the diagnosis of the liver injury (note that the ALT ‘1’ input in the present experiment corresponds to 2 U·mU⁻¹ characteristic of severe LI).

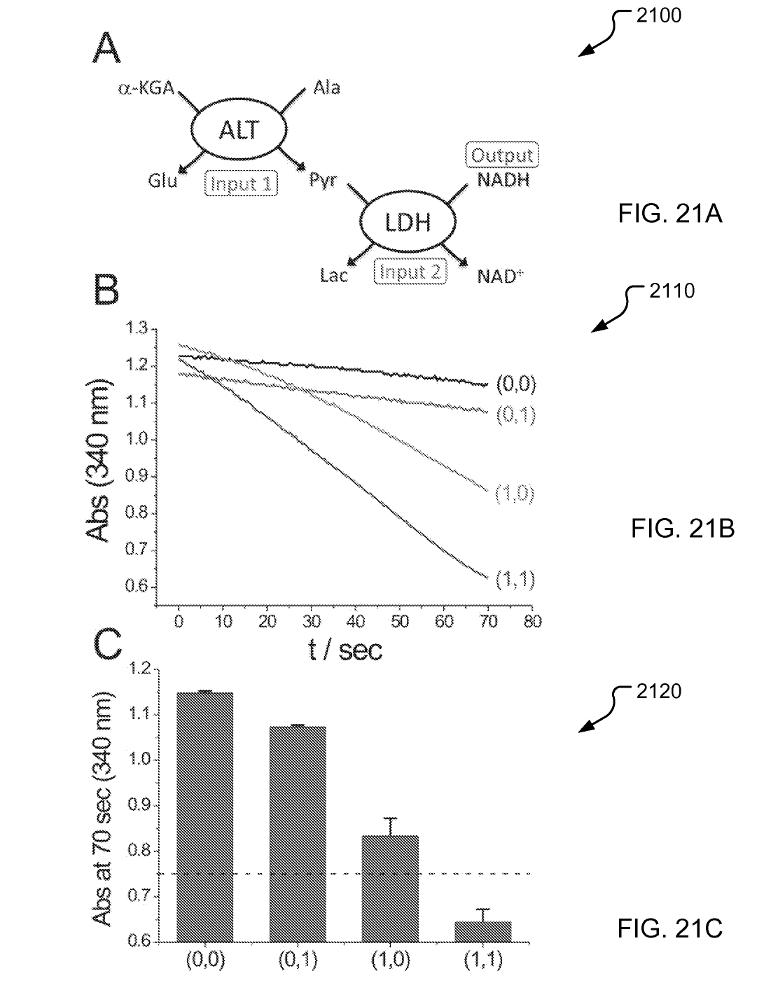

A similar experiment performed with the ALT ‘1’ input of 0.2 U·mL⁻¹ typical of mild LI can allow the correct diagnosis as well due to the well-separated ‘0’ and ‘1’ output signals as shown in FIG. 21. Logic analysis of the liver injury (LI) conditions are shown in FIG. 21. FIG. 21A shows a biocatalytic cascade 2100 applied for the realization of the NAND logic gate activated by ALT (Input 1—corresponding to mild LI) and LDH (Input 2). FIG. 21B shows absorbance changes 2110 corresponding to the consumption of NADH upon operation of the logic system in the presence of different combinations of the input signals. FIG. 21C shows a bar chart 2120 showing the NADH absorbance after 70 sec of the biocatalytic reaction activated with different combinations of the biomarker-input signals. The dashed line is the threshold separating the logic ‘0’ and ‘1’ output values. The normal ‘0’ and pathological ‘1’ concentrations of the input signals are summarized in Table 1. All experiments were performed in human serum solutions mimicking real biomedical samples. A sampling time of 20 sec was selected to yield the best separation between the logic ‘0’ and ‘1’ output signals.

Soft Tissue Injury (STI)

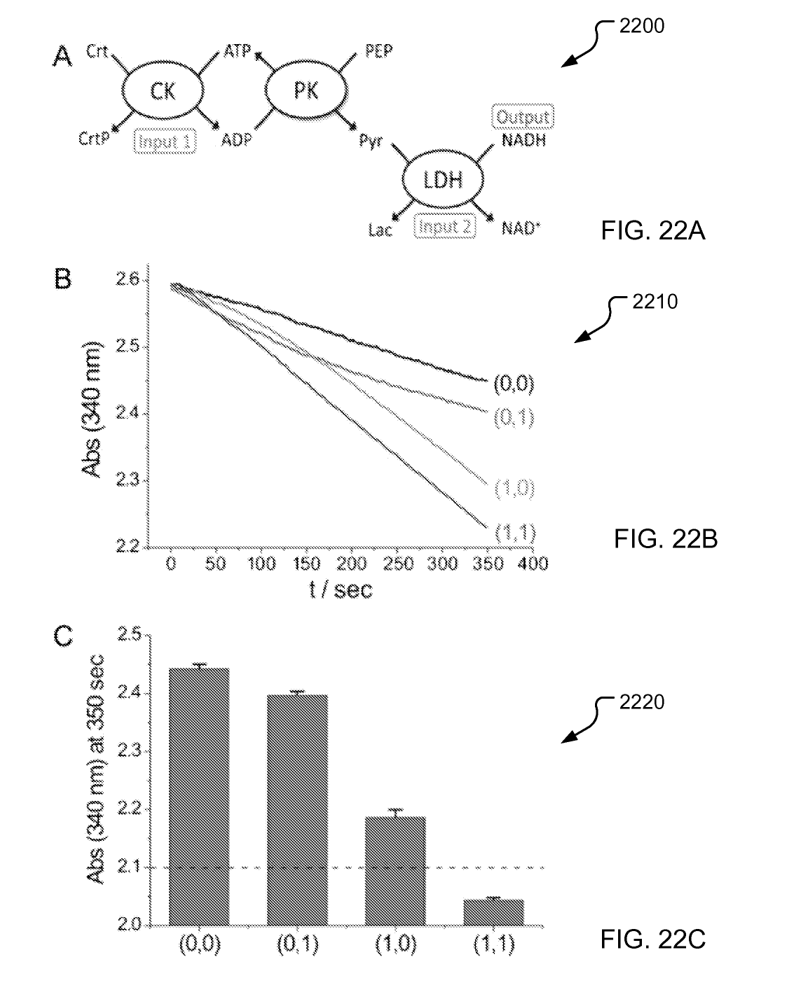

Two enzymes, CK and LDH, were applied as biomarkers characteristic of soft tissue injury. Their simultaneous increase from normal to pathological concentrations (see Table 5 (1900)) can provide an evidence of STI conditions. The biochemical cascade catalyzed in the presence of the both enzyme-biomarkers (note the biocatalytic operation of PK being a part of the logic gate “machinery”) can result in the oxidation of NADH to NAD⁺ 2200 as shown in FIG. 22A, thus yielding the corresponding absorbance decrease (2210) as shown in FIG. 22B. The logic value of the output signal changes from the high ‘1’ value to the low ‘0’ value only upon the concerted work of the both enzyme-inputs (logic inputs combination ‘1,1’), thus mimicking NAND logic operation. Since the logic ‘0’ values of the input signals are not physical zero concentrations (they rather correspond to the normal physiological concentrations of the enzymes), the NADH absorbance is also changing upon other combinations of the inputs (‘0,0’; ‘0,1’; ‘1,0’). However, a threshold at the absorbance of 2.1 measured at the sampling time of 350 sec allows good differentiation of the logic ‘0’ and ‘1’ output values (2220) measured below and above the threshold, respectively as shown in FIG. 22C. Thus, the optical output signal measured below an absorbance of 2.1 allows the diagnosis of the soft tissue injury. The sampling time of 350 sec was optimized to yield the best separation between the logic ‘0’ and ‘1’ output signals. The logic system operation was not performed in a mixture (1:1 v/v) of human serum with a Gly-Gly buffer with the optimized pH and added Mg²⁺ and Kt⁺. In some implementations, the real serum samples can be used with the optimized buffer solution in a microfluidic system.

Abdominal Trauma (ABT)

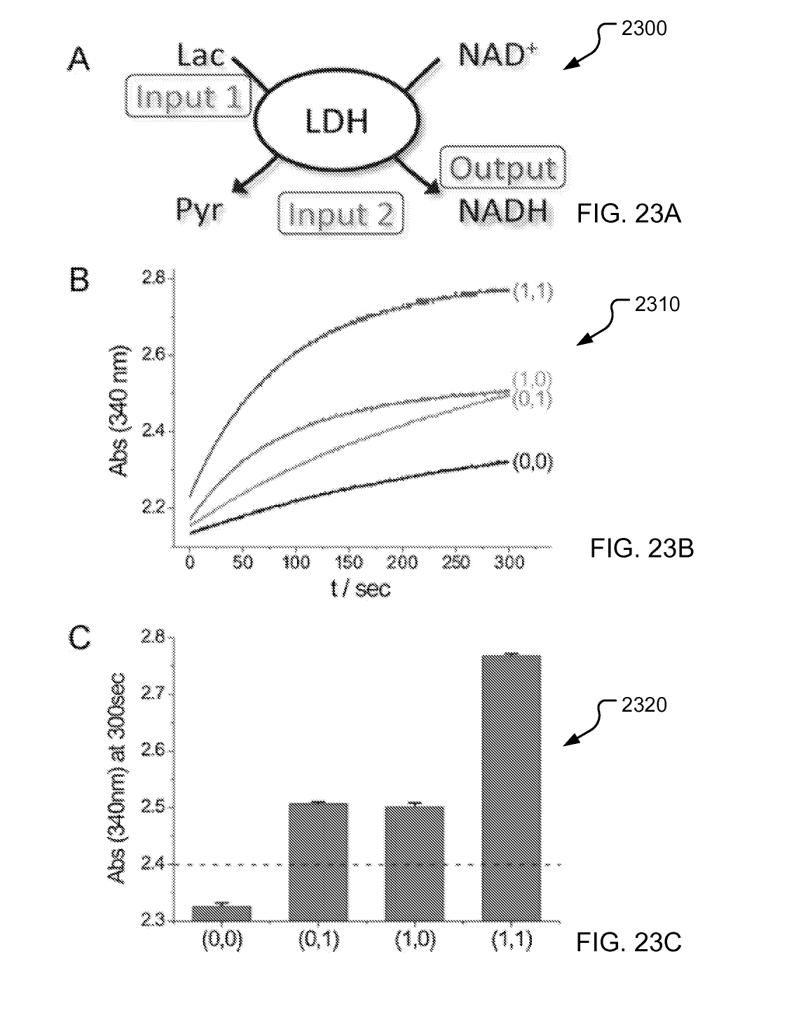

The enzyme LDH and its substrate Lac appearing together at elevated concentrations (see Table 5 (1900) in FIG. 19) can be used as biomarkers of ABT. The biocatalytic reaction activated by the enzyme and the corresponding substrate, can result in the concomitant reduction of NAD⁺ cofactor (2300) as shown in FIG. 23A, thus leading to increased absorbance at λ=340 nm (2310) corresponding to the formation of NADH as shown in FIG. 23B. The logic value of the output signal changes from the low ‘0’ value to the high ‘1’ value only upon the concerted work of the both inputs (logic inputs combination ‘1,1’), thus mimicking AND logic operation (FIG. 4C). Since the logic ‘0’ values of the input signals are not physical zero concentrations (they correspond to the normal physiological concentrations of the enzyme and its substrate), the NADH absorbance is also changing upon other combinations of the inputs (‘0,0’; ‘0,1’; ‘1,0’). However, a threshold at the absorbance of 2.4 measured at the sampling time of 300 sec allows good differentiation of the logic ‘0’ and ‘1’ output values (2320) measured below and above the threshold, respectively as shown in FIG. 23C. Thus, the optical output signal measured above absorbance of 2.4 allows the diagnosis of the abdominal trauma. Despite the fact that the system is rather simple its low-noise operation is challenging and requires optimization. The sampling time of 300 sec was optimized to yield the best separation between the logic ‘0’ and ‘1’ output signals. Similarly to the STI system, we were not able to realize the logic gate operation in a pure serum solution and a mixture (1:1 v/v) of human serum with a Gly-Gly buffer with the optimized pH has been applied.

Interferants in Serum

Numerous compounds present in the serum samples can potentially interfere with the enzymatic “machinery” of the analytical systems described above. Pyruvate is an intermediate product in the LI and STI detection systems. In the LI system pyruvate is generated as an intermediate product by the first system input, ALT enzyme. In the STI system it is also produced by CK/PK biocatalytic cascade. In both systems the pyruvate production corresponds to the presence of one of the biomarker inputs, ALT or CK, respectively. However, pyruvate is present in blood serum (approx. 40 μM) and it could provide a false positive signal even in the absence of the first inputs. Similar complication may occur for ADP in the STI system. Since lactate is a common blood constituent, with its concentration significantly elevated by majority of traumatic injuries, it can prevent conversion of pyruvate to lactate and readout of the output signal corresponding to the decrease of the NADH concentration. In order to suppress the LDH-induced biocatalytic conversion of lactate to pyruvate for the proper performance of the STI system, the solution pH value should be optimized. Another potential complication can originate from the presence of various ions in human serum samples. CK enzyme, which is a key component for the STI system, can be inhibited by many bivalent cations (Ca²⁺, Zn²⁺ and Cu²⁺) and anions (Cl⁻ and PO₄³⁻). In order to achieve an adequate performance of the enzymes for the STI gate, Mg²⁺ ions were added to the solution for preventing the CK inhibition. Additionally, K⁺ ions were added to the reaction mixture to enhance the PK and CK activities. Thus, the 1:1 buffer-serum mixture was used for the analysis of STI and ABT.

The operation of all three logic systems presented in this study was examined using different samples of serum. Some minor sample-to-sample absorbance variations were observed, mostly due to the difference in the transparency of the serum samples. However, the robust operation of the bioanalytical systems always allowed convenient discrimination of ‘0’ and ‘1’ output signals, thus providing reliable diagnostics of the injury conditions.

Chemicals and Reagents

Alanine transaminase from porcine heart (ALT, E.C. 2.6.1.2), pyruvate kinase from rabbit muscle (PK, E.C. 2.7.1.40), creatine kinase from rabbit muscle (CK, E.C. 2.7.3.2), lactate dehydrogenase from porcine heart (LDH, E.C. 1.1.1.27), serum from human male AB plasma, β-nicotinamide adenine dinucleotide dipotassium salt (NAD⁺), β-nicotinamide adenine dinucleotide reduced dipotassium salt (NADH), L-alanine (Ala), α-ketoglutaric acid (α-KTG), L(+)-lactic acid (Lac), creatine anhydrous (Crt), phospho(enol)pyruvate monopotassium salt (PEP), adenosine 5′-triphosphate disodium (ATP, from bacterial source), glycyl-glycine (Gly-Gly), magnesium acetate (MgAc₂), potassium hydroxide (KOH) were purchased from Sigma-Aldrich and were used as supplied without any pretreatment or purification. Ultrapure water (18.2 MΩ·cm) from NANOpure Diamond (Barnstead) source was used in all of the experiments.

Instrumentation and Measurements

A Shimadzu UV-2450 UV-Vis spectrophotometer (with a TCC-240A temperature-controlled holder and 1 mL PMMA cuvette) was used for all optical measurements. A Mettler Toledo SevenEasy s20 pH-meter was employed for the pH measurements. All optical measurements were performed in temperature-controlled cuvettes at 37±0.2° C. mimicking physiological conditions and all reagents were incubated at this temperature prior measurement.

Composition and Operation of Channels for the Analysis of Injuries.

Liver Injury (LI): Pure human serum was used as a background solution. Ala (200 mM), α-KTG, (10 mM) and NADH (150 μM) were dissolved in this solution to perform the NAND logic operation upon activation with the biomarker inputs. Logic ‘0’ and ‘1’ levels of ALT (0.02 and 2 U·mL⁻¹) and LDH (0.15 and 1 U·mL⁻¹) input signals were applied to the system in order to realize circulating levels of these biomarkers at a normal physiological level and an elevated pathological concentration corresponding to the severe liver injury, respectively [35]. In an additional set of experiments (see Supplementary data) another level of the input signal ‘1’ for ALT was applied, 0.2 U·mL⁻¹, corresponding to a mild liver injury condition. The output signal corresponding to the decreasing concentration of NADH was measured optically at λ=340 nm [36].

Soft Tissue Injury (STI): Gly-Gly buffer, 50 mM, containing 6.7 mM MgAc₂ was titrated with KOH to the pH value of 8.5 (note that Mg²⁺ and K⁺ cations are essential for activation of CK and PK, respectively). The following components of the biosensing system were dissolved in the Gly-Gly buffer: NADH (0.2 mM), ATP (1 mM), PEP (0.5 mM), PK (2 U·mL⁻¹), Crt (15 mM) to perform the NAND logic operation upon activation with the biomarker inputs. Then the biosensing system prepared in Gly-Gly buffer was diluted (1:1 v/v) with human serum to mimic biological samples. Logic ‘0’ and ‘1’ levels of CK (0.05 and 0.355 U·mL⁻¹) and LDH (0.075 and 0.5 U·mL⁻¹) input signals were applied to the system in order to realize meaningful circulating levels of these biomarkers under normal physiological and pathological injury conditions, respectively [37]. Immediately following the mixing, optical absorbance measurements were recorded continuously at λ=340 nm, monitoring the decreasing concentration of NADH.

Abdominal Trauma (ABT): Gly-Gly buffer, 50 mM, containing 6.7 mM MgAc₂ was titrated with KOH to the pH value of 8.5. Then NAD⁺ (10 mM) was added to the solution to perform the AND logic operation upon activation with the biomarker inputs. Then the biosensing system prepared in Gly-Gly buffer was diluted (1:1 v/v) with human serum to mimic biological samples. Logic ‘0’ and ‘1’ levels of Lac (0.8 and 3.0 mM) and LDH (0.075 and 0.5 U·mL⁻¹) input signals were applied to the system in order to realize circulating levels of these biomarkers characteristic of normal physiological and pathological injury conditions, respectively [37,38]. The output signal corresponding to the NADH formation was measured optically at λ=340 nm. The obtained results demonstrate reliable operation of multi-analyte biosensor systems with the digital output in human serum solutions mimicking real biomedical samples. These results provide necessary background for transferring the enzyme-logic experiments to the in vivo analysis of the injury biomarkers. By offering a unique “decision” making (YES/NO) feature, the present systems can potentially compete with established immunoassays, without any need of labeled reactants. The coupling of on-body digital biosensor systems with on-demand drug-delivering chemical actuators could also be realized using real biological fluids.

Multi-Enzyme Logic Network Architectures for Assessing Injuries: Digital Processing of Biomarkers

In another aspect, described is a multi-enzyme biocatalytic cascade system that processes simultaneously five biomarkers characteristic of traumatic brain injury (TBI) and soft tissue injury (STI). The system can operate as a digital biosensor based on concerted function of 8 Boolean AND logic gates, resulting in the decision about the physiological conditions based on the logic analysis of complex patterns of the biomarkers. The system represents a multi-step/multi-enzyme biosensor with the built-in logic for the analysis of complex combinations of biochemical inputs. The described techniques, systems and apparatus is based on enzyme-based biocomputing systems that can be used to develop digital biosensor networks.

The described techniques, systems and apparatus can increase the complexity of biomarker-analyzing systems by concatenating many logic operations represented by a multi-enzyme system capable to process many variable biomarker signals. Bi-modal way of action is enabled by a built-in SWITCH feature activated by the presence or absence of switching inputs in specific gates. Switching between two different sub-systems can provide a solution for controlling the biochemical pathways and performance correlation for all multiple AND gates in the system. The built-in SWITCH feature can enable analysis of different physiological conditions within a biochemical logical system operating in a ‘single-pot’ solution. The described system can be applied for the analysis of biomarkers characteristic of the two common injuries: traumatic brain injury (TBI) and soft tissue injury (STI).

Multi-Enzyme Biocatalytic Cascade & Logic Network Systems

The biocomputing approach to multi-signal processing biosensors with built-in logic was exemplified by a system analyzing complex patterns of biomarkers originating from injury conditions. When an injury occurs due to mechanical or chemical damage to specific organs or tissues, chemical species (proteins and low molecular compounds), normally present only in intracellular compartments, are released into various body fluids. Therefore, the rapid and sensitive detection of these biomarkers can be an essential method to obtain a proper injury diagnosis. One disadvantage when detecting enzyme markers with the aim of injury diagnosis is the low-specificity of the biomarkers due to the various pathological conditions underlying their release. However, the specificity of the enzyme-based diagnostic system can be achieved via the implementation of logic analysis of complex biomarker patterns. Instead of detecting a single injury biomarker, a biochemical system composed of enzyme logic gates can process two (or eventually several) physiologically relevant inputs with a corresponding detectable output. The diagnostic capabilities of the biochemical detection system are enhanced when multiple logic gates are concatenated to increase the information processing capacity of the logic system. Hence, such systems can be tuned in order to detect specific injuries while diminishing the possibility of false alarms.

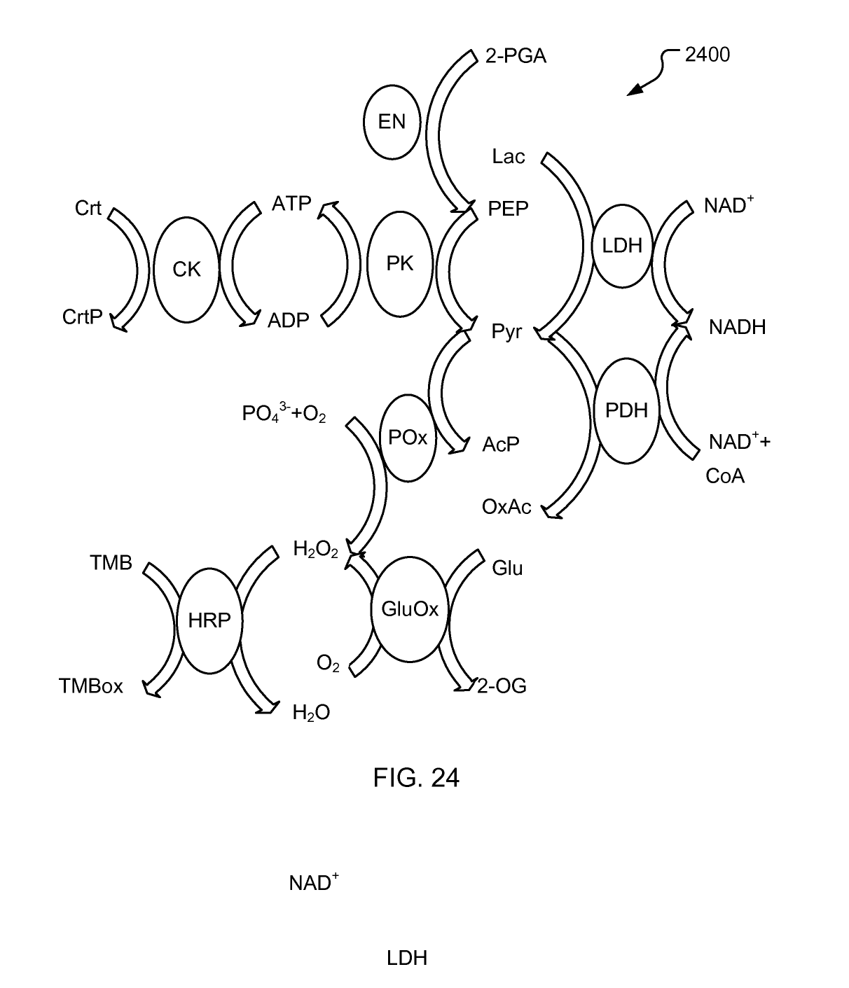

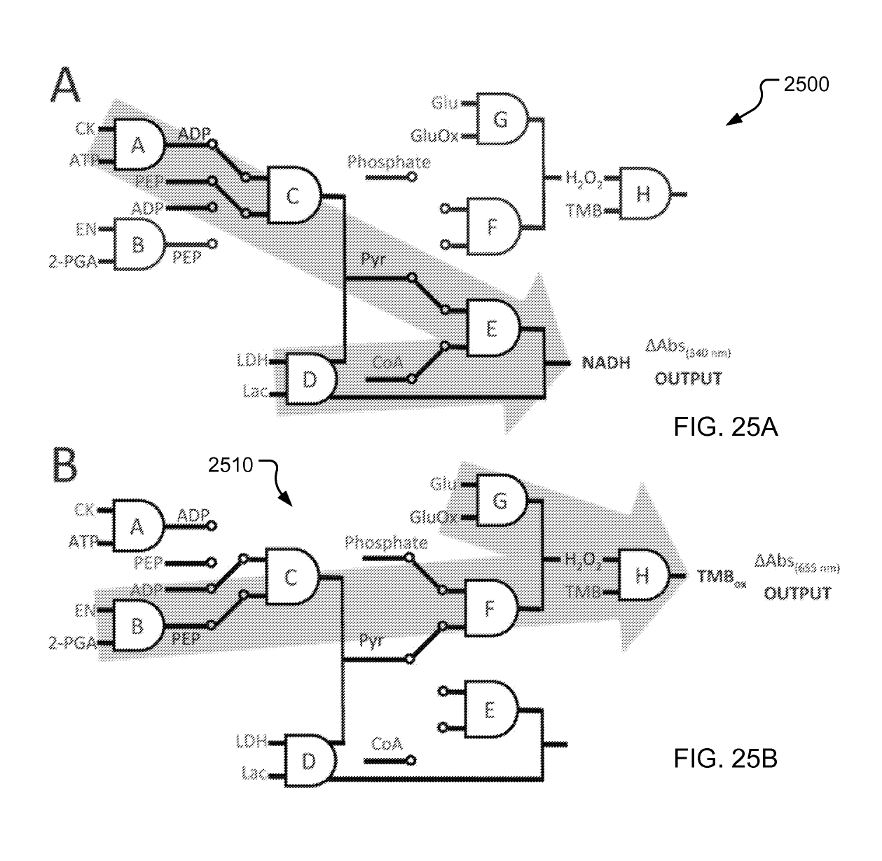

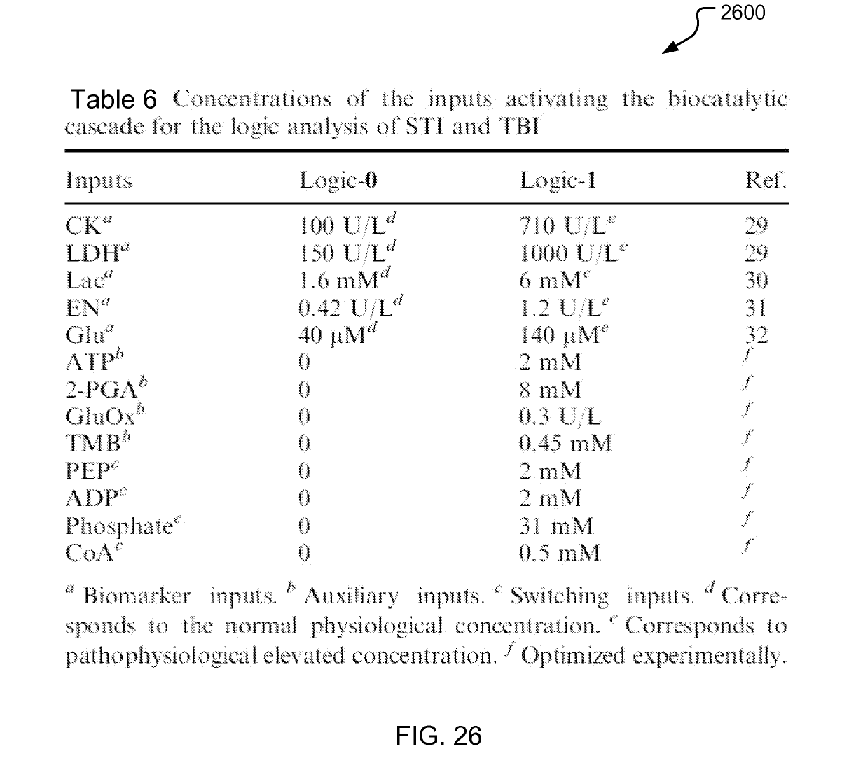

The assessment of two common battlefield injuries, soft tissue injury (STI) and traumatic brain injury (TBI), was performed by an exemplary multi-enzyme biocatalytic cascade 2400 as shown in FIG. 24, which can be described using exemplary comprehensive concatenated logic systems 2500 and 2510 as shown in FIGS. 25A and 25B. The logic networks 2500 and 2510 represented in FIGS. 25A and 25B are equivalent to the biocatalytic cascade 2400 depicted in FIG. 24. The logic networks 2500 and 2510 provide another way of describing the biocatalytic cascade 2400, which is more convenient for the discussion of the system operation. The system architecture of the logic networks 2500 and 2510 can include multiple (8 in the example shown in FIGS. 25A-B) networked logic gates (e.g., AND type logic gates). The biocatalytic system can operate in two different modes: one for the analysis of TBI and another for the analysis of STI with the possibility to switch between them. The system was designed to analyze 5 different biomarkers appearing in different combinations and to lead to a logic conclusion about the presence or absence of TBI or STI conditions. Three biomarkers: creatine kinase (CK), lactate dehydrogenase (LDH) and lactate (Lac) corresponded to the physiological conditions characteristic of STI, while two other biomarkers: enolase (EN) and glutamate (Glu) were reporting on the TBI diagnosis. These five biomarkers were applied as biochemical input signals activating the logic network at two different concentration levels: logic-0 corresponded to the normal physiological concentrations of the biomarkers, while logic-1 was selected at the elevated pathophysiological concentrations corresponding to the respective injuries as shown in Table 6 (2600) in FIG. 26.

Simultaneous processing of many biomarkers through the complex biocatalytic cascade can be accomplished by optimizing the biocatalytic reactions by tuning the reaction rates in order to have comparable output signals for various combinations of the biomarker inputs. The optimization can be achieved by careful selection of the auxiliary inputs: ATP, 2-PGA, glutamate oxidase (GluOx), and TMB. In order to digitize the logic network operation, the auxiliary inputs can be applied at two levels: logic-0 corresponded to the physical zero concentration, while logic-1 was selected experimentally upon optimization of the system ash shown in Table 6 (2600). The auxiliary inputs did not provide any information about physiological conditions related to the injuries, but they were used for optimal processing of the biomarker inputs. When they were applied at “0” levels the system was mute and insensitive to the biomarker inputs, while their application at “1” levels provided optimized processing of the biomarker inputs applied in different combinations of “0” and “1” logic levels. Four additional inputs: PEP, ADP, phosphate and CoA can be used to switch the system operation between the TBI and STI modes. These inputs can be applied at logic-0 levels corresponding to the physical zero concentrations and logic-1 levels being experimentally optimized as shown in Table 6 (2600).

STI Operation Mode of the Logic Network

Since none of the used biomarkers is specific enough for the STI diagnosis, simultaneous appearance of all three STI-related biomarkers (CK, LDH and Lac) at logic-1 values can be used to provide the reliable conclusion about the STI conditions. To implement the multiple biomarkers simultaneously, the system can be adapted as the biochemical representation shown in FIG. 25A with the STI pathway highlighted. The biocatalytic reaction of CK (STI biomarker) and ATP (gate A) results in the production of ADP. Further reaction of ADP with PEP biocatalyzed by PK (gate C) results in the formation of Pyr, which then reacts in the presence of CoA and PDH (gate E) to yield the reduced NADH considered as the output signal. The pathway composed of A-C-E gates is activated only when the biomarker input CK, auxiliary input ATP and switching inputs PEP and CoA appeared at logic-1 values. Simultaneous application of the LDH and Lac inputs (STI biomarkers) at logic-1 (gate D) results in the reduction of NAD⁺ and further increase of the NADH output. This pathway results in the concomitant production of Pyr, which passes through gate E to produce one more equivalent of NADH, thus further amplifying the output signal.

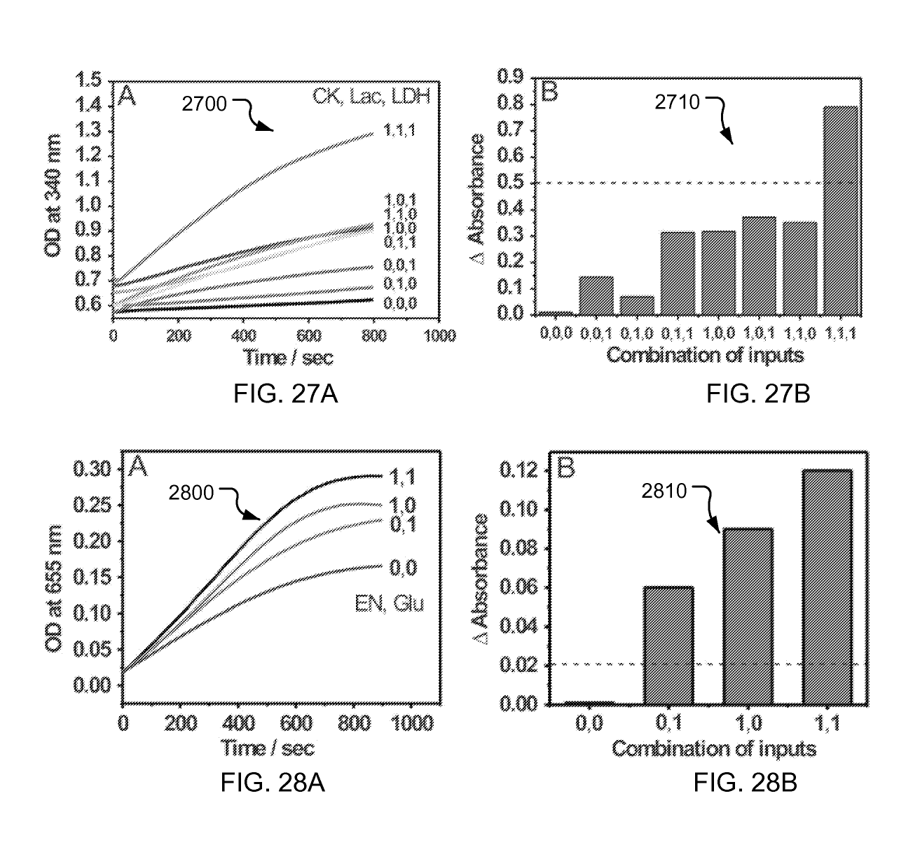

FIG. 27A shows the optical changes in the system measured at λ=340 nm (NADH absorbance) (2700) for different combinations of the biomarker inputs. Simultaneous application of all three biomarker inputs at logic-1 values (input combination 1,1,1) results in the high optical absorbance changes allowing an unambiguous conclusion about the STI condition. The experimentally derived threshold level of 0.5 O.D. allows perfect separation of the logic-0 and 1 levels for the output signal being different at least by 2-fold (2710) as shown in FIG. 27B. In all measurements shown in FIGS. 27A and 28B, the auxiliary (ATP) and switching (PEP and CoA) inputs can be applied at logic-1 values to allow optimal performance of the analytical pathway. At the same time, the switching inputs ADP and phosphate can be applied at logic-0 levels to inhibit the alternative TBI pathway.

TBI Operation Mode of the Logic Network

Opposite to STI, the TBI biomarkers (EN and Glu) are rather specific and can report on the injury presence even appearing alone. A generic EN enzyme was used instead of neuron specific enolase released from damaged brain. The system can be adapted to operate as the logic equivalent circuitry of the biochemical representation shown in FIG. 25B with the TBI pathway highlighted. The biocatalytic reaction of EN (TBI biomarker) and 2-PGA (gate B) results in the formation of PEP. The next step includes the reaction of PEP with ADP (switching input) biocatalyzed by PK (gate C) and results in the production of Pyr. Further reaction of Pyr with phosphate (switching input) biocatalyzed by POx (gate F) yields H₂O₂ which reacts with TMB in the presence of HRP (gate H). This reaction results in the oxidation of TMB and produces the absorbance increase at λ=655 nm characteristic of TMBₒₓ being considered as the final output signal from the pathway composed of B-C-F-H gates. The same signal can be generated through another pathway composed of G and H gates. The biocatalytic reaction of Glu (TBI biomarker) and GluOx results in O₂ reduction and yields H₂O₂ (G gate). Then H₂O₂ reacts with TMB in the presence of HRP to yield TMBₒₓ with the respective absorbance changes (H gate). Careful optimization of the system (by tuning the concentrations of the auxiliary inputs) allows comparable output signals to be produced in both pathways 2500 and 2510.

FIG. 28A shows the optical changes (2800) in the system measured at λ=655 nm (TMBₒₓ absorbance) for different combinations of the biomarker inputs. Any or both biomarkers appearing at logic-1 level results in high absorbance changes signaling about TBI conditions.

The experimentally derived threshold level of 0.02 O.D. allows perfect separation of the logic-0 and 1 levels for the output signal being significantly different for the input combination 0,0 and all other combinations (0,1; 1,0; 1,1) as shown in FIG. 28B. In all measurements shown in FIGS. 28A and 28B, the auxiliary (2-PGA, TMB and GluOx) and switching (ADP and phosphate) inputs can be applied at logic-1 values to allow optimized performance of the analytical pathway. At the same time, the switching inputs PEP and CoA can be applied at logic-0 levels to inhibit the alternative STI pathway.

Switching Between the STI and TBI Modes

Operation of the logic system in the STI mode results in production of ADP as a product of the biocatalytic reaction at gate A. Further reaction of ADP in gate C calls for the presence of PEP which is not produced by the system when it operates in the STI mode. The gate B is mute because of the absence of EN input. Therefore PEP should be added artificially in order to activate gate C. Opposite to this, when the system operates in the TBI mode, PEP is produced in situ in gate B, while ADP which is also needed for the operation of gate C is missing. Gate A is muted because of the absence of CK. In this case, ADP should be added artificially in order to activate gate C. For activation of the network in the STI mode, the switching inputs PEP and ADP should be applied at the logic-1 and 0 values, respectively, while for the TBI mode they should be at the opposite 0 and 1 values. This switch allows the use of gates C and F in two different modes of operation. Similarly, in order to switch between two operational modes, gates F and E should be selectively activated by the correct concentrations of phosphate and CoA. Specifically, phosphate and CoA can be applied at logic-0 and 1 levels in the STI mode and at logic-1 and 0 levels in the TBI mode.

The TBI was operated in 31 mM potassium phosphate buffer, pH 7.58, containing 6.7 mM magnesium sulfate, while the STI mode was realized in 50 mM triethanolamine buffer, pH 7.4, containing 0.2 mM magnesium acetate. In some implementations, the system can operate in the same environment but in different switchable modes.

Chemicals and Materials

The enzymes and other biochemicals were obtained from Sigma-Aldrich and used without further purification: creatine kinase from rabbit muscle (CK, E.C. 2.7.3.2), pyruvate kinase from rabbit muscle (PK, E.C. 2.7.1.40), pyruvate dehydrogenase from porcine heart (PDH, E.C. 1.2.4.1), lactate dehydrogenase from porcine heart (LDH, E.C. 1.1.1.27), enolase from bacterial yeast (En, E.C. 4.2.1.11), pyruvate oxidase from Aerococcus sp. (POx, E.C. 1.2.3.3), peroxidase from horseradish type VI (HRP, E.C. 1.11.1.7), glutamate oxidase from Streptomyces sp. (GluOx, E.C. 1.4.3.11); creatine anhydrous, L-cysteine hydrochloride monohydrate (L-Cys), bovine serum albumin (BSA), thiamine pyrophosphate (TPP), adenine 5′-triphosphate sodium salt (ATP), phosphoenolpyruvic acid monopotassium salt (PEP), L(+)-lactic acid (Lac), coenzyme A sodium salt hydrate (CoA), nicotinamide adenine dinucleotide sodium salt (NAD⁺), D(+)2-phosphoglyceric acid sodium salt (2-PGA), thiamine pyrophosphate (TPP), flavin adenine dinucleotide disodium salt hydrate (FAD), adenine 5′-diphosphate (ADP), L-glutamic acid (Glu), 3,3′,5,5′-tetramethylbenzidine dihydrochloride hydrate (TMB). All other standard inorganic salts/reagents were also purchased from Sigma-Aldrich and used as supplied. Ultrapure water (18.2 MΩ·cm) from NANOpure Diamond (Barnstead) source was used in all of the experiments.

Instrumentation and Measurements

A Shimadzu UV-2450 UV-Vis spectrophotometer with a TCC-240A temperature-controlled cuvette holder and 1 mL poly(methyl methacrylate) (PMMA) cuvettes were used for optical measurements. All optical measurements were performed in temperature-controlled cuvettes/cells at 37° C. mimicking physiological conditions and all reagents were incubated at this temperature prior to experimentation.

A graphical representation of the multi-enzyme biocatalytic system operating in a single-pot solution is outlined in FIG. 24. The input concentrations activating the biocatalytic system are summarized in Table 6 (2600). The system operated in two different modes: (i) for analysis of TBI (tested in 31 mM potassium phosphate buffer, pH 7.58, containing 6.7 mM magnesium sulfate) and (ii) analysis of STI (tested in 50 mM triethanolamine buffer, pH 7.4, containing 0.2 mM magnesium acetate). The constant part of the system (the “machinery” of the logic network) included the following components: PK (10 units/mL), POx (5 units/mL), PDH (2 units/mL), HRP (5 units/mL), creatine (Crt) (15 mM), NAD⁺ (10 mM), TPP (450 μM), L-Cys (3.96 mM), FAD (10 μM) and O₂ (in equilibrium with air). The output signal in the TBI mode was measured as the absorbance increase at λ=655 nm corresponding to the formation of the oxidized form of the redox mediator, TMBₒₓ. The output signal in the STI mode was measured as the absorbance increase at λ=340 nm corresponding to the formation of NADH. The absorbance measurements were started immediately after mixing the reagents in a cuvette and the final absorbance value was taken at 800 sec from the beginning of the reaction. This sampling time was optimal for the effective discrimination of the logic 0 and 1 output signals generated by the system.

Even a very sophisticated multi-enzyme/multi-step biocatalytic cascade can provide reliable diagnostic of physiological conditions upon logic analysis of complex patterns of various biomarkers. The obtained results experimentally confirm that enzyme logic networks with up to 10 concatenated logic gates should be able to process biochemical information within a reasonable noise level. The designed systems exemplify the novel approach to multi-signal processing biosensors mimicking natural biochemical pathways and operating according to the biocomputing concept. In some implementations, biosensor devices can be implemented based on electrochemical methods rather than optical analysis described herein. In addition, biomarkers and their normal and pathophysiological concentrations can be analyzed to reflect specific medical problems. Various bioelectronic devices and bioactuators can be adapted to be controlled by complex patterns of multiple inputs.

Boolean-Format Biocatalytic Processing of Enzyme Biomarkers for the Diagnosis of Soft Tissue Injury

In another aspect, a biocatalytic enzyme logic sensing system is described for the detection of soft tissue injury (STI). The described biocatalytic scheme employs creatine kinase (CK) and lactate dehydrogenase (LDH) as enzyme biomarker inputs to a biochemical cascade that mimics the operational functionality of a NAND Boolean logic gate. Under the optimal conditions, physiological and pathological levels of CK and LDH are detected optically and electrochemically by monitoring the level of reduced nicotinamide adenine dinucleotide (NADH) as an output of the logic gate. The latter technique employs a flexible carbon screen-printed electrode (SPE) to facilitate the on-site detection of STI. By establishing a pathologically meaningful threshold, relatively simple optical and amperometric assays can provide a diagnosis in a straightforward ‘True’/‘False’ digital format. The simultaneous presence of elevated levels of both enzyme inputs can trigger a positive diagnosis. Moreover, an interference investigation can be performed that employs circulating levels of potential interferents. Such an enzyme cascade and enzymatically-processed biochemical information can provide point-of-care injury screening where a rapid determination of pathological situations is a prime consideration.

Techniques, system and apparatus are described for implementing an enzyme logic sensing system for the detection of STI. The described technique merges the inherent redundancy and robustness of a Boolean logic approach in reaching ‘True’ or ‘False’ decisions with the specificity and dynamic range associated with biocatalytic processing. In this manner, multiple biomarkers of acute injury can be integrated and processed in a logical fashion to yield additional physiological information even in the presence of interfering compounds and unpredictable temporal concentration profiles of the biomarker inputs. Such a scheme also lends itself to established analytical electrochemical techniques in addition to harnessing the noise-mitigating capabilities of advanced digital signal post-processing algorithms. The resulting high-fidelity diagnostic route can facilitate a timely therapeutic intervention, which will ultimately lead to increased survival rates.

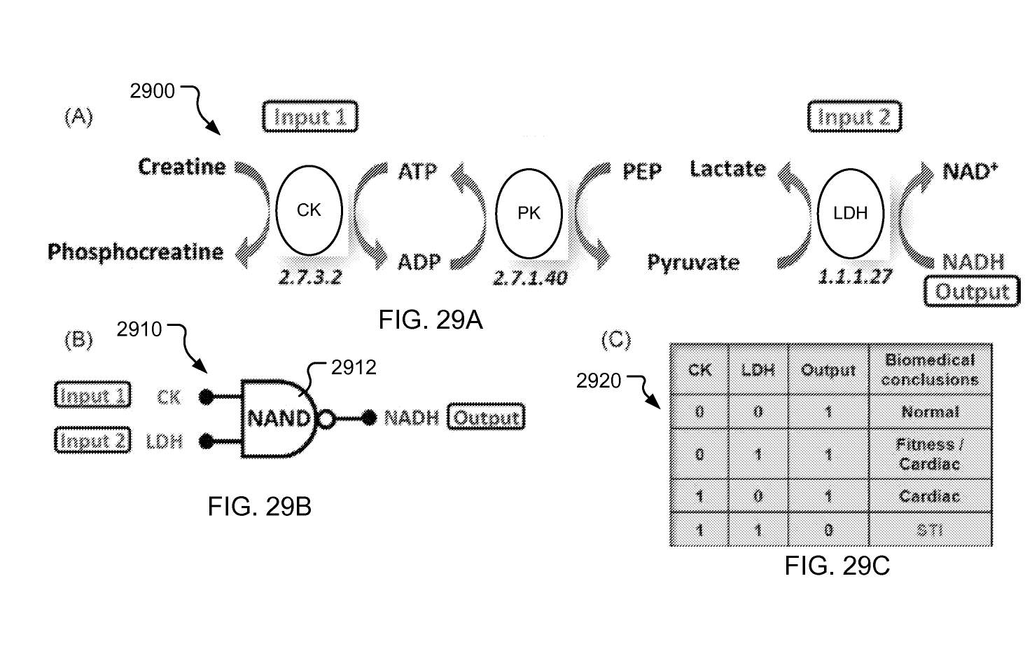

Employing CK and LDH as physiologically relevant enzyme inputs to a Boolean logic gate presents the opportunity to realize a high-fidelity detection concept for STI. Given that both inputs rise upon the incurrence of a meaningful pathological state, reasonable embodiments include the AND and NAND gates, whereby a concomitant increase in both biomarkers following injury can result in an affirmative output. The latter embodiment lends itself to a more straightforward detection system as shown in FIGS. 29A, 29B and 29C. FIGS. 29A, 29B and 29C employ an enzyme cascade of CK, pyruvate kinase (PK) and LDH in combination with their respective substrates: CRTN, ATP, phosphoenolpyruvate (PEP), and reduced nicotinamide adenine dinucleotide (NADH). Moreover, a NAND gate disregards the temporal delay between myocardial-induced rises of CK and LDH, because it's the simultaneous presence of elevated levels of both of these enzymes that can trigger a positive diagnosis. This can negate potential interference that may arise as a result of an acute cardiac condition. The described techniques, system and apparatus represent an exemplary implementation of a logic gate for injury detection using the NAND architecture and an exemplary demonstration of any logic gate operating in the presence of interferents.

The described techniques use the measurements of physiologically relevant levels of CK and LDH processed through an enzyme cascade. Following an initial optical characterization and optimization of the enzyme logic STI machinery, the concept can be evaluated towards amperometric sensing at a disposable carbon screen printed electrode (SPE). The electrochemical assay employs methylene green (MG) as a redox mediator to realize a low-potential detection of the NADH output in the presence of physiologically relevant levels of both enzyme inputs along with potential interferences. In line with the NAND gate topology, a decision threshold is established at a pre-determined current, below which a positive diagnosis is made. This current magnitude, which corresponds to the aggregate concentration of CK and LDH normally found in serum, may be adjusted as required for high-integrity readout under various pathological circumstances. The results presented clearly indicate the potential of the new concept for measuring circulating levels of CK and LDH in the presence of physiologically-relevant interferents, thereby enabling the high-fidelity discrimination between normal (physiological) and abnormal (pathological) STI conditions. Accordingly, the proposed scheme offers great promise for low-cost and rapid decentralized diagnosis of STI.

Enzyme Logic Cascade

FIG. 29A shows an exemplary enzyme logic cascade 2900 employed to realize the NAND gate operation. The equivalent logic gate 2910 is shown in FIG. 29B to include a NAND gate 2912, and a corresponding truth table (2920) shown in FIG. 29C. Using this operation, different combinations of the enzyme biomarker inputs can lead to distinguishable patterns of the NADH output signal. In accordance with the NAND gate operational functionality, logical ‘0’ and ‘1’ levels of CK and LDH input signals, corresponding to normal or anomalous physiological conditions, respectively, ((CK, LDH)=(0,0), (0,1), and (1,0)) can result in an output of logical ‘1’. On the other hand, logical ‘1’ levels of both CK and LDH (1,1) can cause the output state to change from ‘1’ to ‘0’, indicating the occurrence of STI. The logic output signal ‘0’ generated by the NAND gate 2912 and corresponding to the positive STI diagnosis does not imply that the signal is truly at a zero level. Rather, a ‘0’ output may suggest that the system has transitioned from a state producing a signal of high magnitude to one that yields a low-level signal. In this regard, the logic output signal ‘1’ indicates that the output signal is unchanged. The concentration of the reagents in the enzyme cascade can be individually tailored to yield optimal dynamic range between the pathological level (1,1) and normal or anomalous physiological levels (0,0), (0,1), and (1,0). This can enable unambiguous determination of the injury state (when the output signal ‘0’ is generated) due to the establishment of a fixed decision threshold. The simultaneous presence of elevated levels of both enzyme inputs would thus trigger a positive diagnosis. On the other hand, the output signal ‘1’ has undetermined meaning ranging from healthy conditions to various physiological anomalies not related to STI.

Assays for the individual analysis of serum CK and LDH are well-established. Commercially deployed assays for the determination of CK activity typically operate at an alkaline pH (8.8-9.0) as this is the level at which CK exhibits maximum activity. However, assays that have been widely available for quantifying LDH activity operate at neutral pH values (7.2-7.4), the enzyme's optimum range for efficient PYR to LAC conversion. This inherent pH incompatibility presents a unique challenge when endeavoring to employ both CK and LDH as inputs into an enzymatic-processing system, and requires a critical assessment of the optimal pH. A detailed investigation of the potential interferences is also of considerable importance in light of the fact that the enzyme cascade employs several compounds found in body fluids and in view of similar natural biochemical processes occurring within the body.

Optimization of Experimental Parameters

Based on established assay protocols, the concentrations of the constituents of the logic-gate machinery were individually tailored to yield optimal dynamic range between the pathological level (1,1) and normal or anomalous physiological levels (0,0), (0,1), and (1,0). This can allow unambiguous determination of the injury state due to the establishment of a fixed decision threshold. Consistent with commercially available assays, a Gly-Gly buffer solution was identified as the most suitable experimental medium as other buffer solutions operating in similar pH regimes such as phosphate-buffered saline and Tris contained ions that were inhibitory to one or more of the enzymes. Furthermore, the enzyme activator ions Mg²⁺ (required for CK catalysis) and K⁺ (required for PK catalysis) were included in this solution via the addition of MgAc and KOH, respectively. All experiments were performed at physiological temperature (37° C.) due to the sub-optimal performance of the enzyme machinery at room temperature.

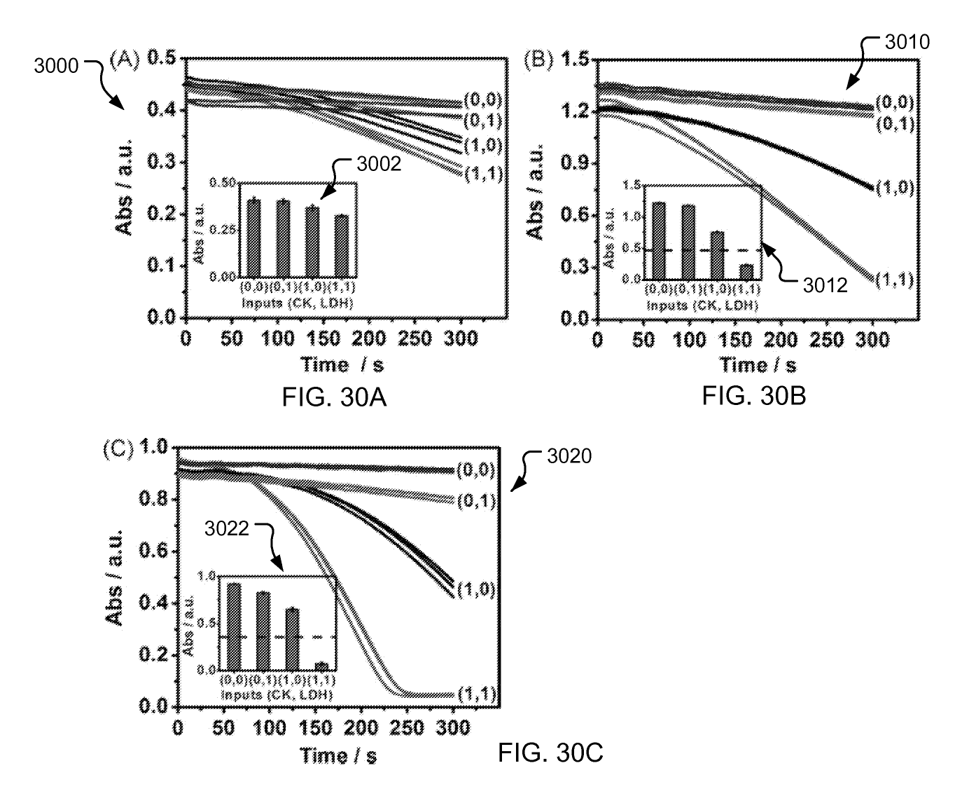

In order to identify the optimal pH that would enable the most favorable operation of the NAND gate, the pH of the Gly-Gly buffer was varied, as shown in FIGS. 30A, 30B and 30C. Commencing experiments with a physiological pH level, the pH value was increased from 7.40 to 8.50. FIG. 30A displays exemplary optical absorbance of the NAND gate at pH 7.40 for three independent experiments at each logic level. In this example, the enzymatic reaction was sluggish to proceed as, little, if any, distinction was observed among the logic levels. The histogram shown in the inset 3002 demonstrates a comparative account of the NAND gate performance at this pH (at 300 sec). In this example, the (1,1) logic level possesses nearly the same absorbance magnitude as the physiological logic levels (1,0), (0,1), and (0,0). Hence, an unambiguous decision threshold could not be established for diagnosis. A further increase of the pH improves the differentiation between the logic levels.

For example, FIG. 30B shows exemplary optical data 3010 obtained at pH 7.95 where a large differentiation between pathological and physiological logic levels is observed, reflecting the faster enzymatic reaction. From the corresponding histogram 3012, the (1,1) logic level was separated by more than 0.52 O.D. from the nearest logic level. At this pH, an explicit decision threshold could be established at 0.49 O.D., leading to highly reliable NAND operation. Proceeding with an even higher pH value of 8.50, which is routinely employed in CK assays, can lead to further enhanced logic gate performance, although not as significant as in the transition from pH 7.40 to pH 7.95.

FIG. 30C displays the optical absorbance (3020) of the NAND gate at pH 8:50. In this case, the conglomerate enzyme reaction proceeded at its fastest rate, with the (1,1) logic state consuming the NADH in its entirety prior to the conclusion of the experiment. The histogram 3022 chronicles the improvement in the NAND gate performance and the increased dynamic range at this pH level. In this case, the (1,1) logic level was extremely well-distanced from the logic level in closest proximity, with a 0.58 O.D. separation between the two states. This enabled an explicit decision threshold to be established at 0.43 O.D., thus allowing for high-fidelity operation. In each measurement, regardless of the operating pH, the (1,0) logic level was is in a closer proximity to (1,1), while the (0,0) and (0,1) logic levels remained largely unperturbed and well-distanced from the two other levels. This reflects the Michaelis-Menten enzymatic kinetics, whereby the PYR substrate generated by physiological as well as pathological levels of CK were at saturating levels for both the LDH ‘0’ and ‘1’ levels, thereby giving rise to a non-proportional relationship. The challenge of physiological monitoring can be resolved in a strip-form embodiment by employing a mild pH adjustment using ‘dry-regent’ alkaline salts. In light of the reduced performance at pH 7.40, all subsequent experiments were performed at a pH of 7.95.

Migrating the NAND Concept to the Electrochemical Domain

To achieve low-cost decentralized screening of STI, the aforementioned protocol has been migrated to the amperometric domain using a disposable SPE. To develop compact analytical devices and based on the results obtained above, the pH of the buffer was established at 7.95 and chronoamperometric measurements were performed for each logic level with MG added to the assay. The redox mediator MG offers a low-potential detection of NADH, hence minimizing potential electroactive interferences. The detection potential was varied between −0.2 V and 0.2 V (vs. Ag/AgCl) in order to determine the optimal potential with the most favorable signal-to-noise ratio (S/N). Likewise, the MG concentration was varied from 100 μM to 10 mM in order to further maximize the S/N. This multivariate parameter sweep indicated that, in the presence of 300 μM of MG, an applied potential of 0.0 V resulted in the highest S/N figure-of-merit.

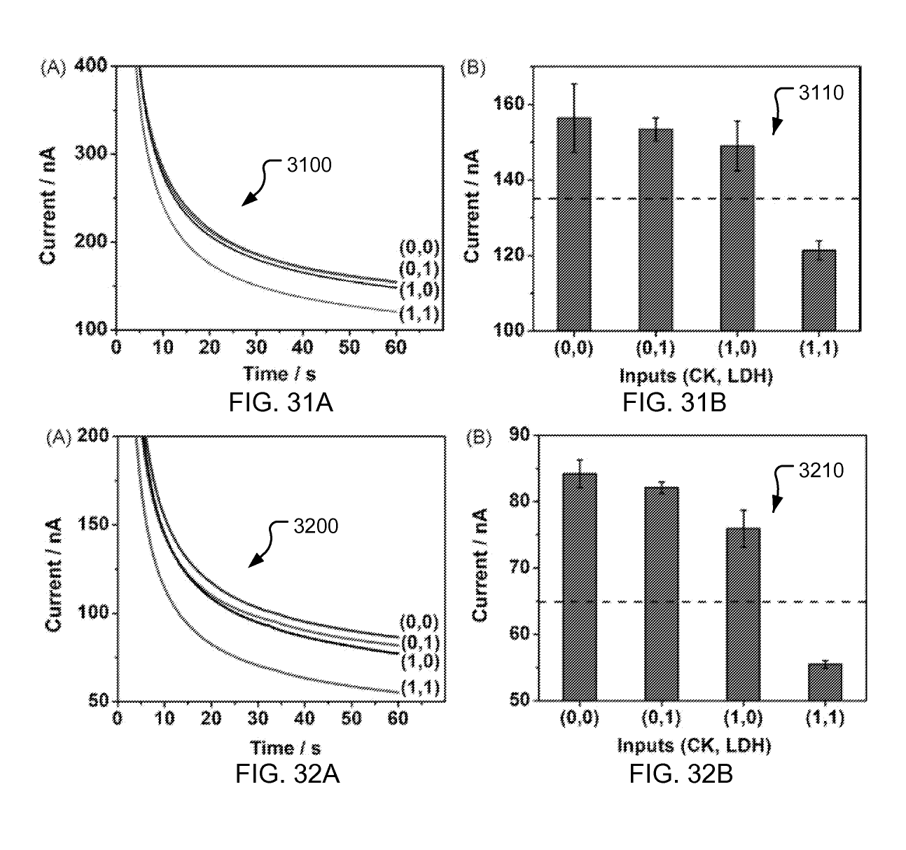

FIG. 31A displays chronoamperograms (using a potential step to 0.0 V vs. Ag/AgCl) (3100) obtained at the carbon SPE by the NAND gate upon application of various input combinations. At 60 sec sampling time, the difference in current (3110) between the (1,1) logic and (1,0) logic levels was 27 nA, as shown in FIG. 31B. As in the optical experiments, the histogram indicates that a straightforward decision threshold could be instituted to realize high-fidelity NAND gate operation. This threshold was fixed at 135 nA.

Accordingly, a good agreement can be observed between the optical and electrochemical data, as indicated from a comparison of the histograms presented in FIGS. 30B and 31B, thereby confirming the validity of the transition of the experimental procedure from the optical to the electrochemical domain. With the electrochemical protocol in functional order, the effect of undesired (yet physiologically relevant) biomarkers and potential interference was subsequently investigated.

Interference Investigation

A biosensor using an enzyme cascade as its backbone, by nature, employs many of the same compounds found within the body and operates under similar biochemical principles. One of the most well-established indicators of muscular fatigue and injury is a physiological rise in serum levels of LAC from 1.6 to 6 mM. Any assay that employs LDH to assess STI should minimize the effect of LAC to yield reliable results. In addition to accounting for the influence of LAC on the operation of the logic gate, the effect of incorporating other physiologically relevant compounds is also a necessity even if they are not outward biomarkers of STI. Of the multitude of substances present in the blood, the most detrimental compounds to the operation of the system are those that also serve as the substrates and co-substrates for the enzyme reactions. Particularly, these interferents could potentially hinder or even reverse the enzymatic reaction, thereby leading to complete operational failure of the logic gate. As such, PYR, PCr, ADP, and NAD⁺ must be employed in the logic gate at their physiological levels in an attempt to emulate realistic sensing conditions.

Utilizing the same assay conditions as stated above, a comprehensive interference examination can be performed with circulating levels of PYR, PCr, ADP, and NAD⁺ as well as pathological levels of LAC. The resulting chronoamperograms are shown in FIG. 32A (average of three independent experiments). The pathological logic level (1,1) was easily distinguishable and separated from the physiological logic levels by greater than 20 nA. The histogram shown in FIG. 32B illustrates the high-fidelity operation of the enzyme logic gate wherein a low standard deviation of less than 3 nA was obtained. With the logic threshold affixed at 65 nA, STI could be readily diagnosed under encumbering, yet realistic conditions. It should be noted that this logic threshold was reduced from the original value 135 nA in the presence of interference. Although the standard deviation did not exceed 5% of the mean value, other intrinsic and practical physiological parameters will dictate the precision of each logic level and the position of the threshold. Such a system is expected to perform as intended for a large majority of the population with CK and LDH levels that fall within clinically-established ranges. Yet, due to the complex nature and variable extent of STI afflictions and of potential interferences, the execution of a large clinical, study that integrates various forms of STI is imperative in order to select the most optimal threshold level for the general population.

The presented interference study underscores the robustness of the enzyme logic approach in assessing a pathologically complex and diverse affliction such as STI. The sizeable dynamic range of the NAND gate in the presence of high levels of interferents highlights the advantages of the concept when contrasted with traditional biosensor approaches. In light of the presence of interferents, the use of Boolean processing and a decision threshold for digital diagnosis enabled the assessment of proper diagnosis with a high degree of confidence under practical and varied conditions.

Preparation of Chemicals and Reagents

Glycylglycine (Gly-Gly), magnesium acetate tetrahydrate (MgAc), potassium hydroxide (KOH), bovine serum albumin (BSA), creatine (CRTN), phosphocreatine disodium salt hydrate (PCr), adenosine 5′-triphosphate disodium salt hydrate (ATP), adenosine 5′-diphosphate sodium salt (ADP), phosphoenolpyruvic acid monopotassium salt (PEP), pyruvic acid (PYR), L(+)-lactic acid (LAC), β-nicotinamide adenine dinucleotide, reduced dipotassium salt (NADH), nicotinamide adenine dinucleotide (NAD⁺), methylene green (MG), pyruvate kinase from rabbit muscle (21,312 U/mL, E.C. 2.7.1.40), creatine kinase from rabbit muscle (417 U/mg, E.C. 2.7.3.2), and lactate dehydrogenase from porcine heart (15,363 U/mL, E.C. 1.1.1.27) were purchased from Sigma-Aldrich and were used as supplied without any pretreatment or purification. Ultra pure deionized water (18.2 MΩ-cm) from a NANOpure Diamond (Barnstead) source was used in all experiments.

Gly-Gly buffer solutions were prepared at 50 mM concentrations with 6.7 mM MgAc to provide the magnesium ion activator for CK. The buffer was then titrated with 1 M KOH to create solutions with pH values 7.40, 7.95, and 8.50 (while providing the potassium ion cofactor essential for PK). All reagents were prepared with this buffer solution.

Instrumentation

A CH Instruments model 1232A potentiostat was used for all electrochemical measurements and a Shimadzu UV-2450 UV-Vis spectrophotometer (with a TCC-240A temperature-controlled cuvette holder and 500 μL quartz cuvettes) was used for all optical measurements. A Mettler Toledo SevenEasy s20 pH-meter was employed for the pH measurements. A VWR Analog Heatblock was utilized as a temperature-controlled incubator.

Electrode Design and Fabrication

The carbon screen printed working electrode consisted of a rectangular carbon working electrode (exposed geometrical area: 4 mm²). The fabrication of the flexible SPE is detailed: A carbon-based ink (Ercon E3449) was printed on a 75 μm-thick Mylar polyester film substrate (DuPont) to define the working electrode geometry using a Speedline Technologies model TF 100 MPM-SPM screen-printer. Subsequent to the printing process, the patterned substrate was cured at 120° C. for 20 min. A silicone adhesive-coated polyester tape (DWrap, CS Hyde) was applied on the electrode surface in order to define its active area. The substrate was finally cleaved to create single-use electrodes possessing overall dimensions of 5 mm×34 mm. All electrochemical measurements were accomplished by employing a 200 μL reaction cell with an external platinum wire counter electrode and a quasi-reversible Ag/AgCl wire reference electrode. The carbon SPE was suspended above the reaction cell such that only the active working electrode area was immersed in the test solution.

Composition of the Logic Gates and Protocol

In order to comply with the 500 μL quartz cuvette, a total of 500 μL of reagents were employed in each optical experiment. This volume consisted of 100 μL of Gly-Gly and 50 μL of each of the reagents: NADH (3 mM), BSA (0.3% w/v), ATP (20 mM), PEP (5 mM), PK (20 kU/L), CRTN (150 mM), along with 50 μL of each of CK and LDH. Logical ‘0’ and ‘1’ levels of CK (100 and 710 U/L) and LDH (150 and 1000 U/L) input signals were applied to the logic system in order to realize meaningful circulating levels of these enzymes. All reagents were dispensed in the cuvette and mixed by inversion. Immediately following mixture, an optical absorbance measurement was recorded continuously for 300 sec at λ=340 nm (at 37° C.).

The electrochemical experiments were conducted by employing a sample volume of 160 μL in each measurement. This volume consisted of 16 μL Gly-Gly buffer and 16 μL of each of the reagents used in the optical experiments along with an MG redox mediator (300 μM). In the interference study, the Gly-Gly buffer was spiked with physiological levels of the following compounds: LAC (6 mM), PYR (40 UV), PCr (150 μM), ADP (130 nM), and NAD⁺ (30 μM). Circulating levels of the substrates and co-substrates already implemented in the enzyme cascade were several orders of magnitude lower than those employed and thus their effect was not considered. All reagents in both the optimization and interference studies (with the exception of MG) were mixed in a vial and incubated at 37° C. in a heatblock for 180 sec. Following this incubation period, MG was added to the solution, which was subsequently mixed and dispensed in the electrode reservoir held at 37° C. A chronoamperogram was then recorded for 60 sec with a stepped potential of 0.0 V (vs. Ag/AgCl).

A novel enzyme logic system based upon the concert operation of an enzyme cascade is designed to process biochemical information for the diagnostic assessment of STI. Such operation offers reliable information processing and generates distinguishable patterns of the NADH output signal arising from various combinations of the enzyme biomarker inputs. Following an optimization of the operating conditions, an interference investigation employing both physiological and pathological concentrations of potential interferents was also performed. The enzymatically-processed biochemical information presented in the form of a NAND truth table allowed for high-fidelity discrimination between normal (physiological) and abnormal (pathological) conditions even under extreme circumstances where interfering compounds were present at their pathological levels. Described is a successful enzyme logic gate operation applied to diagnostic merits in a physiologically relevant environment.

Multiplexing of Injury Codes for the Parallel Operation of Enzyme Logic Gates

In another aspect, described is a highly parallel enzyme logic sensing system employing a novel encoding scheme for the determination of multiple pathophysiological conditions. The described system multiplexes a contingent of enzyme-based logic gates to yield a distinct ‘injury code’ corresponding to a unique pathophysiological state as prescribed by a truth table. The described system is illustrated using an array of NAND and AND gates to assess the biomedical significance of numerous biomarker inputs including creatine kinase, lactate dehydrogenase, norepinephrine, glutamate, alanine transaminase, lactate, glucose, glutathione disulfide, and glutathione reductase to assess soft-tissue injury, traumatic brain injury, liver injury, abdominal trauma, hemorrhagic shock, and oxidative stress. Under the optimal conditions, physiological and pathological levels of these biomarkers can be detected through either optical or electrochemical techniques by monitoring the level of the outputs generated by each of the six logic gates. By establishing a pathologically meaningful threshold for each logic gate, the absorbance and amperometric assays tendered the diagnosis in a digitally-encoded 6-bit word, defined as an ‘injury code.’ This binary ‘injury code’ can allow the effective discrimination of 64 unique pathological conditions to offer a comprehensive high-fidelity diagnosis of multiple injury conditions. Such processing of relevant biomarker inputs and the subsequent multiplexing of the logic gate outputs to yield a comprehensive ‘injury code’ offers significant potential for the rapid and reliable assessment of varied and complex forms of injury in circumstances where access to a clinical laboratory is not viable. While the described system of parallel and multiplexed enzyme logic gates is illustrated here in connection to multi-injury diagnosis, it could be readily extended to a wide range of practical medical, industrial, security and environmental applications.

Techniques, apparatus and system are described for implementing biocomputing ‘injury coding’ diagnostics that can multiplex multiple injuries and assign each pathophysiological state a distinct ‘injury-code,’ thereby enabling highly parallelized operation in the digital domain while minimizing the complexity of the analog electronic integration required for multiple-potentiostat electrochemical devices. Due to the Boolean nature of the enzyme logic concept, all normal physiological states upon implementation of the AND logic operation can be ascribed a logical ‘0’ value in the biochemical domain, which accounts for most of the injury combinations, prior to the transduction of the signals to the electrical domain. Only pathological conditions causing a change in the outputs relative to a pre-defined threshold level would result in a logical ‘1’ value, thereby alleviating the complex decision routines that must be performed in the electronic domain. The inverted logic values ‘1’ and ‘0’ can be applied for normal and pathological conditions, respectively, upon application of the NAND logic operation. As such, n outputs can be multiplexed into an n-bit word or ‘injury code’ for a comprehensive assessment of health conditions. A unique ‘injury code’ can thus be ascribed to a specific pathophysiological state in accordance with a truth table. A simple look-up table in digital logic circuitry could thus be employed to determine which injuries, if any, have been sustained in accordance with this distinct sequence of bits. In this manner, an array of n individual dual-input enzyme logic gates (each evaluating a separate injury) can assess 2ⁿ possible pathological conditions among 2²ⁿ possible physiological states. The described techniques represents successful implementation of parallelization of enzyme logic gates applied to diagnostic merits, as well as the simultaneous multiplexing of the outputs of multiple logic gates in the biochemical domain into a binary injury code.

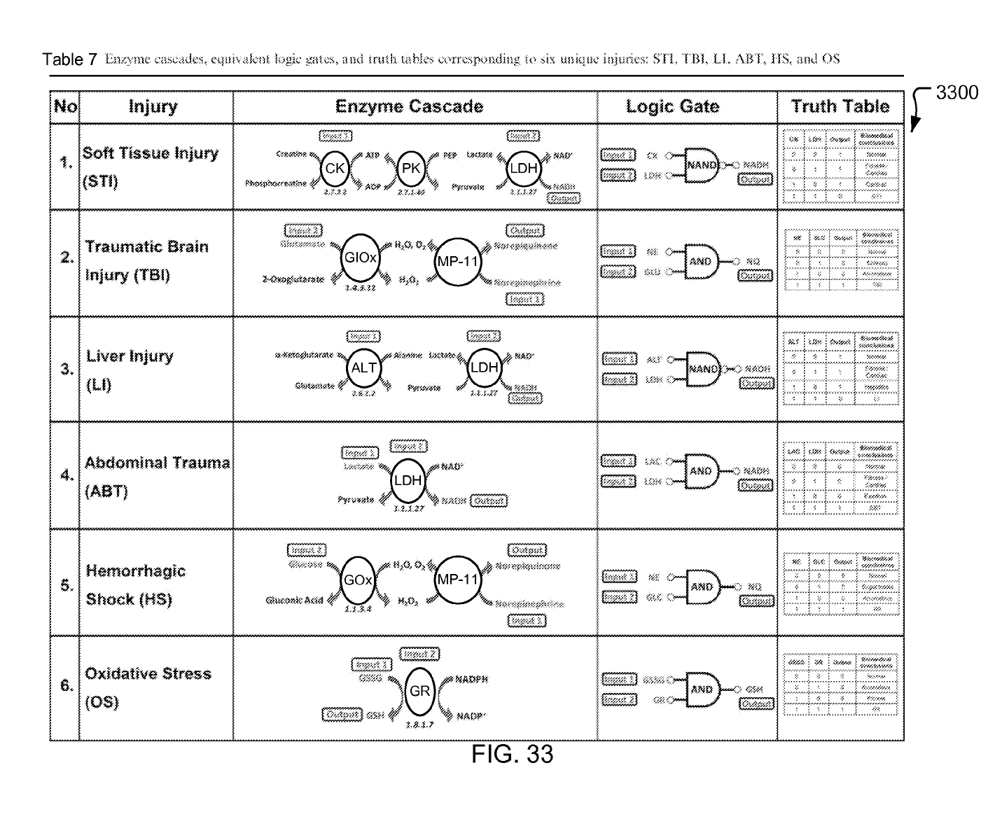

FIG. 33 is a table 3300 showing enzyme cascades, equivalent logic gates, and truth tables corresponding to six unique injuries: STI, TBI, LI, ABT, HS, and OS. To provide a rapid and reliable diagnosis of multiple injury states, an array of two NAND and four AND enzyme logic gates can be assembled as shown in table 7 (3300). Six different pathological conditions are assessed including soft tissue injury (STI), traumatic brain injury (TBI), liver injury (LI), abdominal trauma (ABT), hemorrhagic shock (HS), and oxidative stress (OS) using twelve biomarker inputs. Optical absorbance and amperometric characterization of the six-gate system are conducted in order to verify compliance with a truth table as well as to ensure proper differentiation between the logical ‘0’ and ‘1’ output levels. The outputs of the six logic gates were subsequently multiplexed to yield a distinct 6-bit injury code representing 64 unique pathological conditions among 4096 possible physiological scenarios. This leads to an additional (comprehensive) level of information on the overall nature of the injury, beyond the assessment of individual injuries performed by the individual gates. The system integration of clinically relevant enzyme logic gates and the subsequent multiplexing of their outputs into a cohesive injury code thus offer great promise for the rapid, reliable, and decentralized assessment of multi-injury and polytrauma conditions that typically occur in the battlefield (compared to traditional biosensing schemes). The system includes separate channels individually tailored for specific biomedical needs. The modularity of the system allows for straightforward reconfiguration of the constituents to enable the device to adapt, expand and meet new requirements and applications. The potential of the new modular biocomputing coding concept extends beyond the diagnosis of multiple injuries, as the concept could be readily extended for reliably assessing a wide range of other practical real-world scenarios involving multitude changes.

Parallel and multiplexed enzyme logic gates are described in connection to multi-injury diagnosis. Such biocomputing-based comprehensive diagnosis of multiple injury conditions can harness the processing capabilities of six individual enzyme logic gates, realizing NAND and AND operations, for the detection of STI, TBI, LI, ABT, HS, and OS using clinically relevant combinations of the biomarkers CK, LDH, NE, GLU, ALT, LAC, GLC, GSSG, and GR. The output of each logic gate was subsequently integrated as one of the constituents of the injury code-generating system to enable high-fidelity assessment of multiple injury conditions and hence a comprehensive analysis of the scope of injury.

Upon the optimization of the design parameters, the four unique combinations of the two biomarker inputs to each individual logic gate results in distinguishable patterns of its respective output. For two of the gates, and in accordance with the NAND gate operational functionality, logical ‘0’ and ‘1’ levels of the input biomarkers, corresponding to normal or anomalous physiological conditions, respectively, ((Input 1, Input 2)=(0,0), (0,1), and (1,0)), results in an output of logical ‘1’. On the other hand, logical ‘1’ levels of both of the biomarkers (Input 1, Input 2)=(1,1) causes the output state to change from ‘1’ to ‘0’, indicating that an injury has occurred. The logic output signal ‘0’ generated by the NAND gate and corresponding to a positive diagnosis does not imply that the signal is truly at a zero level. Rather, a ‘0’ output implies that the system has transitioned from a state producing a signal of high magnitude to one that yields a low level signal. In this regard, the logic output signal ‘1’ indicates that the output signal is unchanged. The remaining four AND gates functioned under a logical inversion of the above operation. In this manner, logical ‘0’ and ‘1’ levels of the input biomarkers, corresponding to normal or anomalous physiological conditions, respectively, ((Input 1, Input 2)=(0,0), (0,1), and (1,0)), resulted in an output of logical ‘0’ whereas logical ‘1’ levels of both of the biomarkers (Input 1, Input 2)=(1,1) caused the output state to change from ‘0’ to ‘1’, thereby signifying that an injury has been sustained. Overall, the digital outputs of the six logic gates can be subsequently multiplexed to yield a comprehensive 6-bit injury code (e.g., 0, 0, 1, 1, 0, 1), corresponding to 64 unique pathological conditions (i.e., various combinations of six unique injuries).

With respect to each logic gate, the concentrations of the reagents that served as machinery (and not as inputs) were individually tailored to yield optimal dynamic range (DR) between the pathological level (1,1) and normal or anomalous physiological levels not related to injuries (0,0), (0,1), and (1,0). This can allow unambiguous determination of the injury state (when the output signal ‘0’ is generated by the NAND gate and ‘1’ by the AND gate) due to the establishment of a fixed decision threshold. With respect to both gate archetypes, simultaneous presence of elevated levels of both inputs would trigger a positive diagnosis. On the other hand, the output signal ‘1’ in the NAND topology and ‘0’ in the AND embodiment imply extraneous pathophysiological states ranging from healthy conditions to various physiological anomalies not related to the injuries under investigation.

Application of logic ‘0’ and ‘1’ input values corresponding to the physiologically relevant concentrations of the injury biomarkers required substantial optimization of the enzyme-based logic gates. For some of the injury scenarios, the concentration differential between logic ‘0’ and ‘1’ values can be quite narrow implying strict limitations to the threshold values. Thus, the design of the logic gate can require a more careful consideration and optimization of the assay parameters compared with logic gates employed in previous studies, whereby logic ‘0’ and ‘1’ values are represented by truly zero and arbitrarily high concentrations of the input signals, respectively.

Soft Tissue Injury (STI)

Soft tissue injuries are among the most pervasive injuries sustained in combat and can be difficult to identify in numerous circumstances. Among clinically established indicators of STI, serum CK and LDH have been routinely employed in the assessment of muscular exertion, fatigue, injury, and trauma.

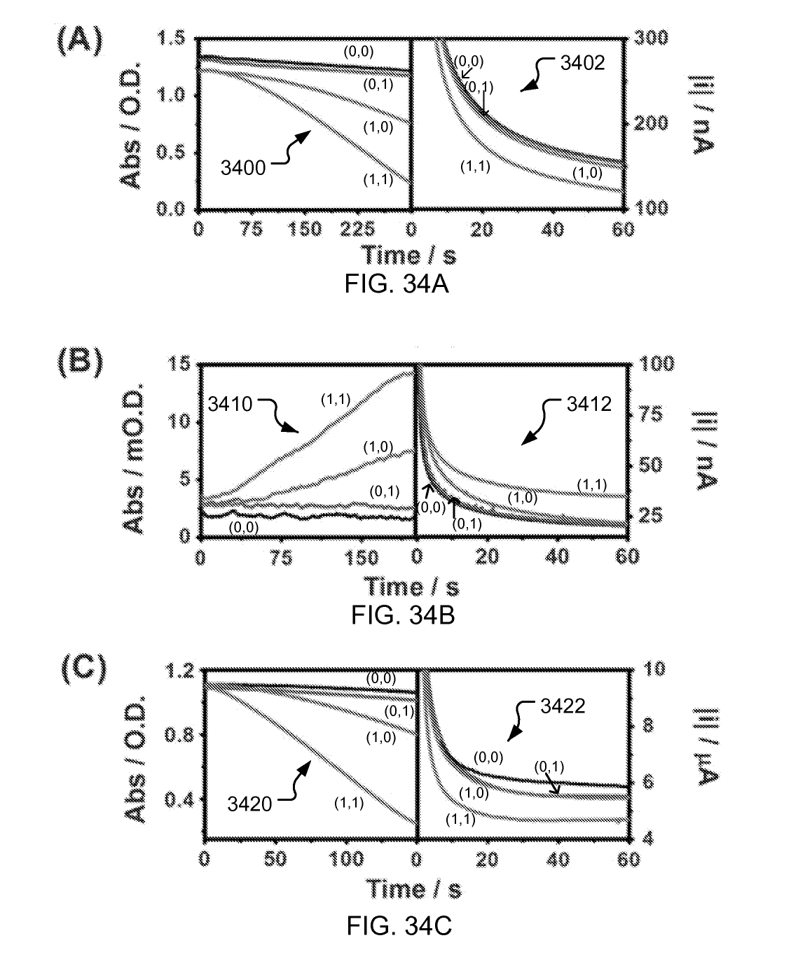

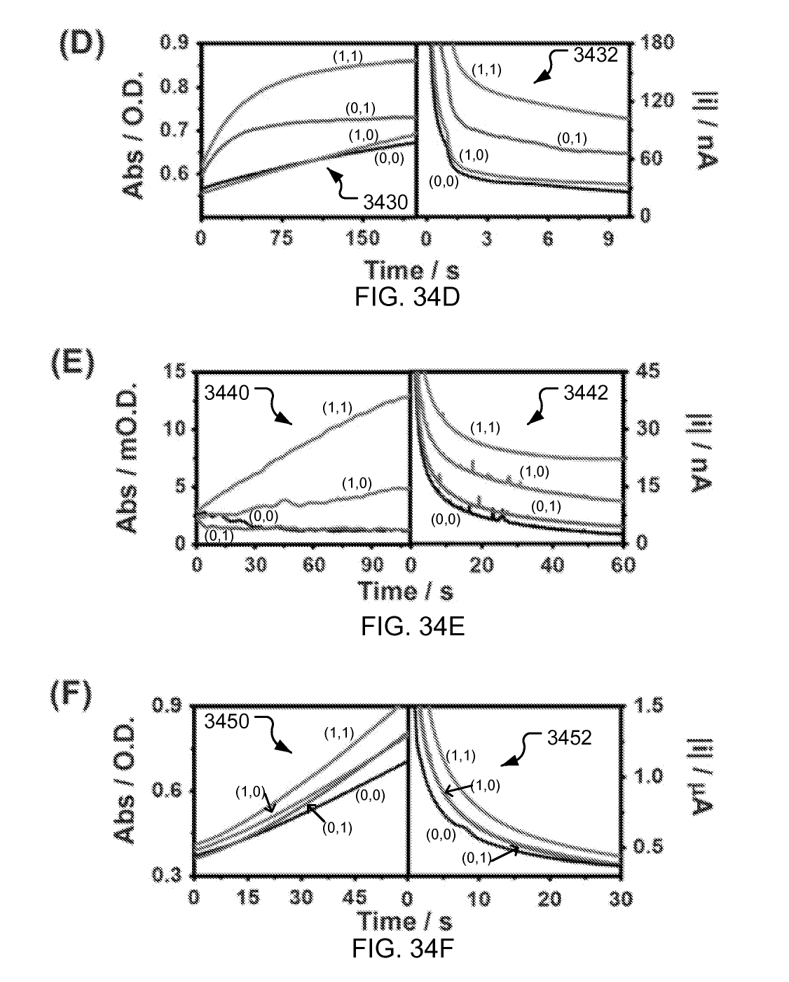

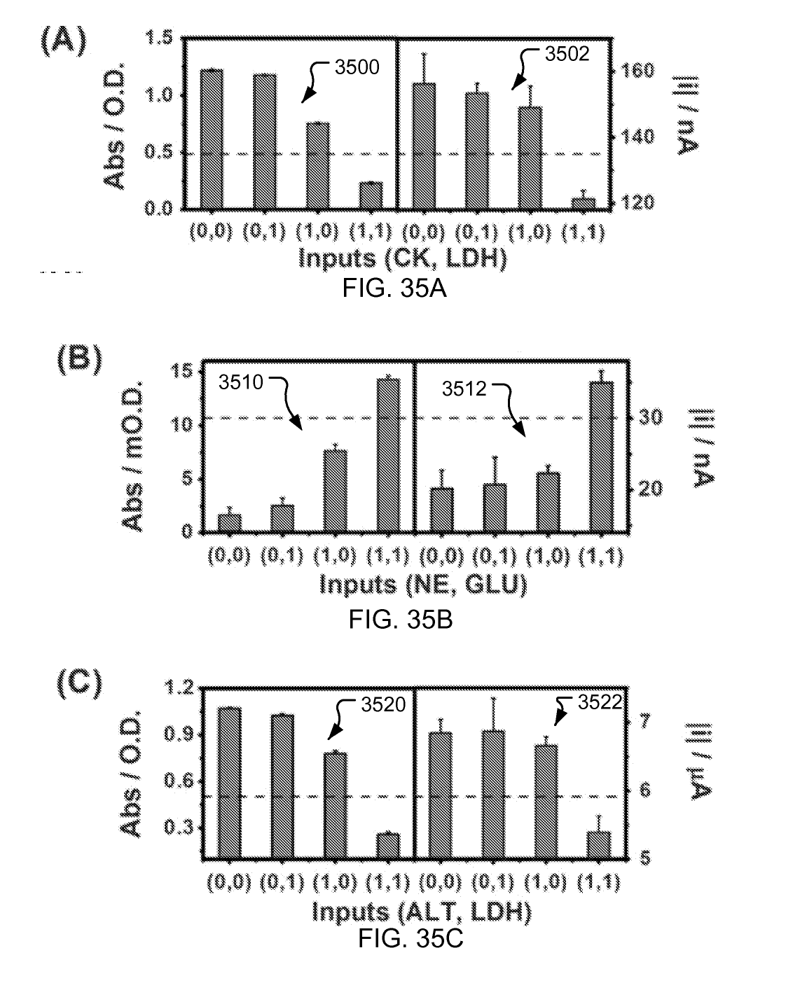

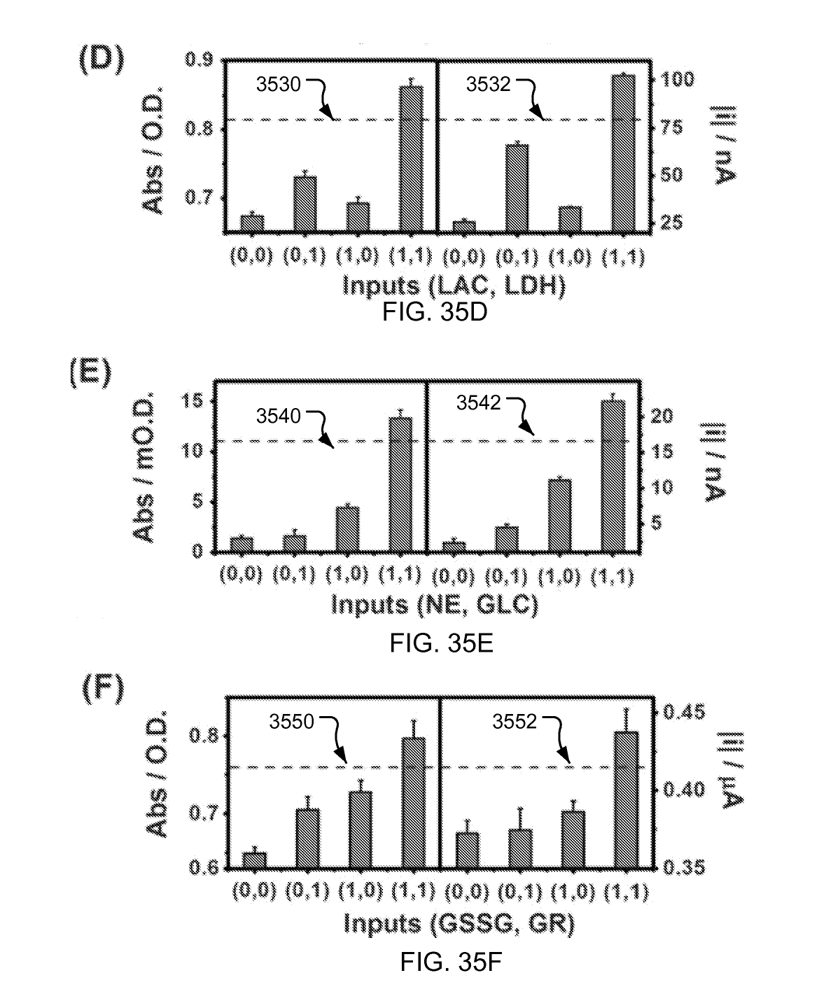

Commencing experiments using optical assay methods, FIG. 34A (left) displays the optical absorbance (3400) of the NAND gate at λ=340 nm. A large differentiation between pathological and physiological logic levels is observed, reflecting the rapid enzymatic reaction. From the corresponding bar chart (3500) as shown in FIG. 35A (left) constructed using the data at 300 sec, the (CK, LDH)=(1,1) logic level was separated by more than 0.52 O.D. from the nearest logic level. An explicit decision threshold could hence be established at 0.49 O.D., leading to highly reliable NAND operation.

In accordance with the goal of low-cost decentralized screening of STI and developing compact analytical devices, the aforementioned protocol was subsequently migrated to the amperometric domain using a disposable SPE. Towards this goal, chronoamperometric measurements were performed for each combination of input signals with MG added to the assay, allowing for the low-potential detection of NADH, hence minimizing potential electroactive interferences. The detection potential was established at 0.0 V to maximize the signal-to-noise ratio (SNR) figure of merit. Chronoamperograms (3402) are shown in FIG. 34A (right) which were obtained at the carbon SPE by the NAND gate upon application of various input combinations. At 60 sec sampling time, the difference in current between the (CK, LDH)=(1,1) logic and (1,0) logic levels was 27 nA, as shown by the bar chart (3502) as shown in FIG. 35A (right). As in the optical experiments, the bar chart (3502) indicates that a straightforward decision threshold could be instituted to realize high-fidelity NAND gate operation. This threshold was fixed at 135 nA.

Traumatic Brain Injury (TBI)

Traumatic brain injuries are among the most debilitating injuries suffered in the battlefield and have garnered much recent attention due to the wide range of symptoms and characteristics presented by individuals suffering from the condition. NE, a catecholamine hormone neurotransmitter, and GLU, an amino-acid excitatory neurotransmitter, are among the most widely-employed indicators of neurological damage and trauma.

Commencing experiments using optical assay methods, FIG. 34B (left) displays the optical absorbance (3410) of the AND gate at λ=487 nm. As with the STI gate, a large differentiation between pathological and physiological logic levels is observed, reflecting favorable enzyme kinetics. From the corresponding bar chart (3510) (FIG. 35B (left)) constructed using the data at 200 sec, the (NE, GLU)=(1,1) logic level was separated by 6.65 mO.D. from the nearest logic level. An explicit decision threshold could hence be established at 10.96 mO.D., leading to highly reliable AND operation.

The aforementioned protocol was subsequently migrated to the amperometric domain using a disposable SPE. Accordingly, chronoamperometric measurements were performed for each combination of the input signals with the concentration of norepiquinone (NQ) serving as the output indicator. The detection potential was established at −0.4 V for SNR considerations. Chronoamperograms (3412) are shown in FIG. 34B (right) which were obtained at the carbon SPE by the AND gate upon application of various input combinations. At 60 sec sampling time, the difference in current between the (1,1) logic and (0,1) logic levels was 13 nA, as shown by the bar chart (3512) in FIG. 35B (right). As in the optical experiments, the bar chart (3512) indicates that a straightforward decision threshold could be instituted to realize high-fidelity AND gate operation, which was established at 30 nA.

Liver Injury (LI)

Screening of liver injury has routinely been employed in the clinical laboratory through enzyme-based assay tests to assess sepsis—a secondary, but life-threatening condition arising due to such injuries. This injury is especially prevalent in combat situations where damaged organs causes foreign matter to enter and circulate in the bloodstream. Serum ALT and LDH have enjoyed widespread use as enzyme biomarkers in such assays and are well-suited for integration as input biomarkers and enzyme backbones to the enzyme logic machinery.

Commencing experiments using optical assay methods, FIG. 34C (left) displays the optical absorbance (3420) of the NAND gate at λ=340 nm. A large differentiation between pathological and physiological logic levels is observed, again a result of enhanced enzyme activity. From the corresponding bar chart (FIG. 35C (left) 3520) constructed using the data at 150 sec, the (ALT, LDH)=(1,1) logic level was separated by 0.52 O.D. from the nearest logic level. An explicit decision threshold could hence be established at 0.52 O.D., yielding the expected NAND operation.

Electrochemical experiments were subsequently executed by employing a bare GC electrode. Accordingly, chronoamperometric measurements were performed for each combination of the input signals and the level of NADH consumed was monitored at 0.8 V. Chronoamperograms (3422) are shown in FIG. 34C (right) obtained at the GC electrode by the NAND gate upon application of various input combinations. At 60 sec sampling time, the difference in current between the (1,1) and (1,0) logic levels was 1.3 as shown by the bar chart (3522) in FIG. 35C (right). As in the optical experiments, the bar chart validates that a decision threshold could be unambiguously implemented to realize high-fidelity NAND gate operation, which was established at 6.0 μA.

Abdominal Trauma (ABT)

As with liver injury, severe abdominal trauma frequently results in sepsis and must be addressed with little or no delay in order to improve survival. In addition, ABT is another example of a common battlefield injury which has been linked to high mortality rates. Serum LAC and LDH are well-established biomarkers of such injury in addition to serving extensive use in assays.

Initially, optical experiments were conducted and FIG. 34D (left) displays the optical absorbance (3430) of the AND gate at λ=340 nm at each combination of the input signals. Pathological and physiological logic levels were easily differentiated, which confirmed that the assay conditions were favorable for this application. From the corresponding bar chart (FIG. 35D (left 3530)) constructed using the data at 200 sec, the (LAC, LDH)=(1,1) logic level was separated by 0.13 O.D. from the logic level in closest proximity. Accordingly, an unambiguous decision threshold could be established at 0.81 O.D. in order to yield AND functionality.

Following optical experiments, electrochemical investigations were then performed by employing a disposable SPE. Chronoamperometric measurements were performed for each combination of the input signals with MG serving as the output mediator to reduce the overpotential required for the detection of NADH. The detection potential was established at 0.1 V to maximize SNR. Chronoamperograms (3432) are shown in FIG. 34D (right) which were obtained at the carbon SPE by the AND gate upon application of various input combinations. At 10 sec sampling time, the difference in current between the (1,1) logic and (1,0) logic levels was 36 nA, as shown by the bar chart (3532) in FIG. 35D (right). Upon examination of the bar chart, it is apparent that a decision threshold could be unambiguously implemented to realize high-fidelity AND gate operation (as in the optical case), which was established at 84 nA.

Hemorrhagic Shock (HS)

Hemorrhagic shock, a condition that arises due to uncontrolled bleeding, is another pervasive example of a high-mortality combat injury and frequently occurs as a result of the infliction of gun-shot wounds and blast injuries. This critical condition must be assessed before the individual who has sustained the injury bleeds to death. Serum GLC and NE have been identified as biomarkers of such injury and can increase many-fold upon presentation of this condition.

Optical experiments were conducted and FIG. 34E (left) displays the optical absorbance (3440) at λ=487 nm of the AND gate for each combination of the input signals. Pathological and physiological logic levels were well-separated, which again confirmed favorable assay conditions. From the corresponding bar chart (FIG. 35E (left 3540)) constructed using the data at 100 sec, the (NE, GLC)=(1,1) logic level was separated by 8.89 mO.D. from the logic level in closest proximity. Accordingly, a straightforward decision threshold could be established at 9.50 mO.D. in order to yield AND functionality.

Following optical experiments, electrochemical investigations were then performed by employing a disposable SPE. Chronoamperometric measurements were performed for each combination of the input signals with the concentration of norepiquinone (NQ) serving as the output indicator. The detection potential was established at −0.4 V for SNR considerations. Chronoamperograms (3442) are shown in FIG. 34E (right) which were obtained at the carbon SPE by the AND gate upon application of various input combinations. At 60 sec sampling time, the difference in current between the (1,1) logic and (1,0) logic levels was 11 nA, as shown by the bar chart (3542) in FIG. 35E (right). In accordance with these results, the decision threshold to realize high-fidelity AND gate operation (as in the optical case) was established at 16 nA.

Oxidative Stress (OS)

Oxidative stress refers to a broad scope of pathological states and accompanies nearly all forms of physical stress or strain experienced by the body including those acquired in battle. GSSG and GR mitigate the body's biochemical response to oxidative stress and elevations in each compound in serum have been found to be associated with such events. Thus, GSSG and GR are excellent candidates for biomarkers that can enable highly reliable logic gate operation.

Optical experiments were first conducted and FIG. 34F (left) displays the optical absorbance (3450) of the AND gate at λ=412 nm for each combination of the input signals using DTNB for optical analysis of the GSH produced in situ. Pathological and physiological logic levels were easily differentiable, thereby validating high-fidelity operation. From the corresponding bar chart (FIG. 35F (left 3550)) constructed using the data at 60 sec, the (GSSG, GR)=(1,1) logic level was separated by 0.07 O.D. from the logic level in closest proximity. A decision threshold could hence be set at 0.76 O.D. in order to yield AND functionality.

Electrochemical investigations were then performed by employing a disposable SPE. Chronoamperometric measurements were performed for each combination of the input signals with Co-PC serving as the output mediator to reduce the overpotential required for the detection of GSH. The detection potential was established at 0.5 V to maximize SNR. Chronoamperograms (3452) are shown in FIG. 34F (right) which were obtained at the CoPC-modified carbon SPE by the AND gate upon application of various input combinations. At 30 sec sampling time, the difference in current between the (1,1) logic and (1,0) logic levels was 51 nA, as shown by the bar chart (3552) in FIG. 35F (right). As this figure illustrates, a decision threshold could be implemented to realize high-fidelity AND gate operation (as in the optical case), and was accordingly established at 412 nA.

Systems Integration

Accordingly, good correlation was observed between the optical and electrochemical data in each of the six separate experiments, as indicated from a comparison of the bar charts presented in FIGS. 35 A-F (3500, 3502, 3510, 3512, 3520, 3522, 3530, 3532, 3540, 3542, 3550, 3552) thereby confirming the validity of the transition of the experimental procedure from the optical to the electrochemical domain. With the electrochemical protocol in functional order, the output of each logic gate could be integrated as one of the constituents of the injury code-generating system.

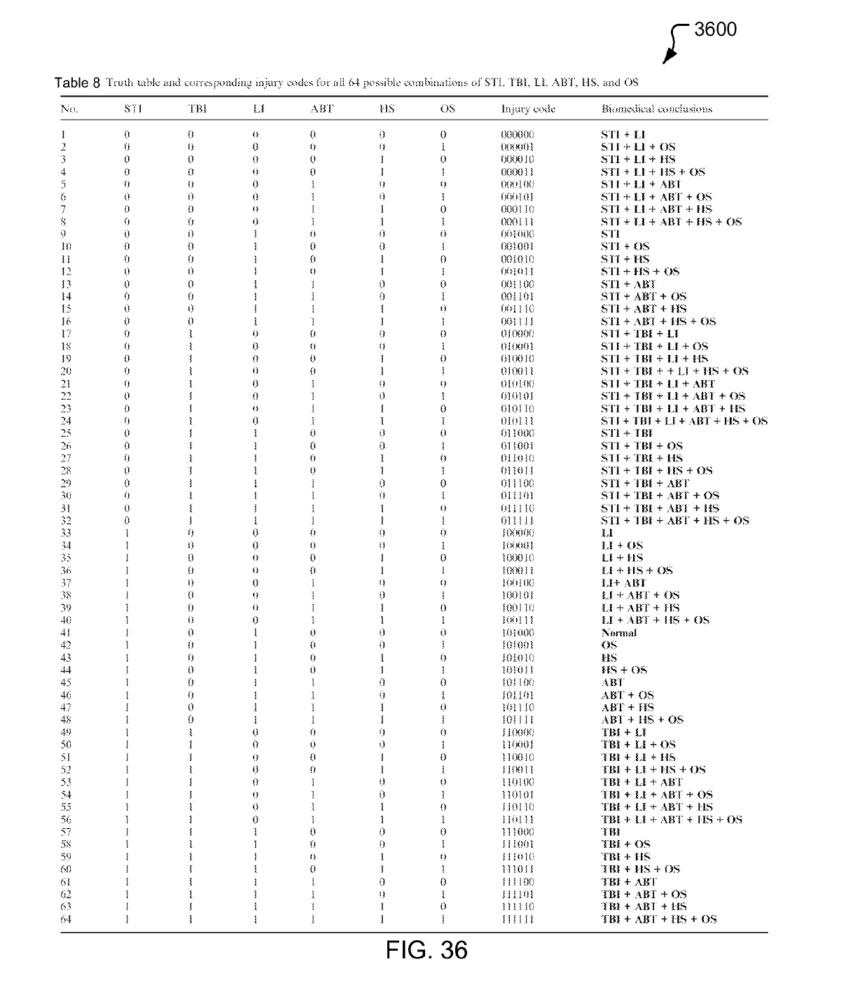

With the above six gates optimized to enable high-fidelity detection, a 6-bit injury code was concatenated from the outputs of each of the individual gates. Accordingly, 2⁶=64 unique injury combinations could be ascertained, as shown in Table 8 (3600) of FIG. 36, among 2¹²=4096 possible pathophysiological scenarios. A comprehensive injury code could thus be constructed to account for various combinations of six unique injuries.

Such an encoding scheme enables a reduced dependence on analog circuitry required for the synthesis of high-integrity waveforms required for digital signal processing, as the outputs are generated with substantial differentiation between the digitally defined physiological and pathological levels in the biochemical domain. Moreover, the binary nature of the output indicators of injury enabled multiplexing operations to be performed directly in the digital domain and could be appraised against a lookup table for reliable evaluation of pathophysiological state. The sizeable dynamic range and high noise margins of the NAND and AND logic gates facilitates such high-fidelity operation and serves to further underscore the advantages of the concept when contrasted with traditional biosensor and lab-on-a-chip approaches. Without the use of Boolean processing and the establishment of a concomitant decision threshold for digital diagnosis, a reliable assessment of injury could not be tendered nor could an injury code be constructed. In absence of these merits, the approach would encounter serious obstacles in discarding the noise arising from extraneous interferents and anomalous pathophysiological conditions and extracting the signal of interest. Therefore, the operational merits of the logic gate architecture enabled the establishment of an unambiguous decision threshold and digital manipulation of the biomarker signals, thereby enabling the concatenation of an inclusive injury code to assess multiple injury conditions, which allowed the system an enhanced ability to detect injury when compared with traditional biosensing concepts.

Noise reduction, fault tolerance, robustness and scalability are all factors to be considered when attempting to improve the performance of enzyme-based information processing systems. Experimental optimization was implemented in order to produce a reliable threshold between output=0 and output=1. Although noise can be suppressed by logic network design, other intrinsic experimental parameters will dictate the precision of each logic level. When assessing the optical and electrochemical experimental results, the standard deviation for most of the values does not exceed 5% of the mean value, demonstrating reproducibility within the chosen physiological and pathological input range. The chosen threshold lines separating logic output=0 and logic output=1 varied between 11% and 60% of the mean value; hence we can consider that the logic gates serve as reliable injury predictors for at least 90% of the repetitions.

Preparation of Chemicals and Reagents

Enzymes including glutathione reductase from S. cerevisiae (GR, E.C. 1.6.4.2), glucose oxidase type X-S from Aspergillus niger (GOx, E.C. 1.1.3.4), alanine transaminase from porcine heart (ALT, E.C. 2.6.1.2), pyruvate kinase from rabbit muscle (PK, E.C. 2.7.1.40), creatine kinase from rabbit muscle (CK, E.C. 2.7.3.2), and lactate dehydrogenase from porcine heart (LDH, E.C. 1.1.1.27) were purchased from Sigma-Aldrich and were used as supplied without any pretreatment or purification. L-glutamate oxidase from Streptomyces sp. (GlOx, E.C. 1.4.3.11) was obtained from Yamasa Corporation, Japan, and used as supplied. All other chemicals (Sigma-Aldrich) were used without purification: bovine serum albumin (BSA), β-D-(+)-glucose (GLC), (−)-norepinephrine (NE), microperoxidase (MP-11), β-nicotinamide adenine dinucleotide dipotassium salt (NAD⁺), β-nicotinamide adenine dinucleotide reduced dipotassium salt (NADH), β-nicotinamide adenine dinucleotide 2′-phosphate reduced tetrasodium salt (NADPH), L-glutamic acid (GLU), L-alanine (L-ALA), α-ketoglutaric acid (KTG), L(+)-lactic acid (LAC), L-glutathione disulfide (GSSG), creatine anhydrous (CRTN), phospho(enol)pyruvate monopotassium salt (PEP), adenosine 5′-triphosphate disodium (ATP, from bacterial source), glycyl-glycine (Gly-Gly), cobalt(III)-phthalocyanine (CoPC), magnesium acetate tetrahydrate (MgAc), sodium hydroxide (NaOH), magnesium chloride (MgCl₂), calcium chloride (CaCl₂), potassium hydroxide (KOH), pyruvic acid (PYR), methylene green (MG), tris(hydroxymethyl)aminomethane hydrochloride salt (Tris-buffer), potassium phosphate monobasic (PPM), potassium phosphate dibasic (PPD), citric acid anhydrous (CA), sodium citrate dihydrate (SC), and 5,5′-dithiobis-(2-nitrobenzoic acid) (DTNB—Ellman's reagent). Ultrapure water (18.2 MΩ cm) from NANOpure Diamond (Barnstead) source was used in all of the experiments.

Instrumentation and Measurements

A Shimadzu UV-2450 UV-Vis spectrophotometer (with a TCC-240A temperature-controlled cuvette holder and 1 mL PMMA or 0.5 mL quartz cuvettes) was used for all optical measurements and a CH Instruments model 1232A potentiostat was used for all electrochemical measurements. A Mettler Toledo SevenEasy s20 pH-meter was employed for the pH measurements. A VWR Analog Heatblock was utilized as a temperature-controlled incubator. All optical measurements were performed in temperature-controlled cuvettes/cells at 37±0.2° C. mimicking physiological conditions and all reagents were incubated at this temperature prior to experimentation. Measurements for the each combination of input signals were repeated at least 3 times.

Electrode Design and Fabrication

A screen printed three-electrode strip, custom-designed using AutoCAD®, consisted of a circular carbon working electrode (geometrical area: 3 mm²) inscribed in hemispherical counter (area: 10 mm²) and reference electrodes (area: 2 mm²). The fabrication of the flexible screen-printed electrode system is detailed: An Ag/AgCl-based ink from Ercon (E2414) was employed to define the conductive underlayer as well as the reference electrode. All potentials are reported vs. this reference electrode. A carbon-based ink (Ercon E3449) was then overlaid on the conductor to define the working and counter electrode geometry. Finally, an insulator ink (Ercon E6165) was overlaid on the Ag/AgCl and carbon layers to insulate all except the contact pads and the upper active segment of the electrodes. A Speedline Technologies MPM-SPM screen printer was used to print the pattern onto a 250 μm-thick flexible polyethylene terephthalate substrate (DuPont Melinex 329). Subsequent to the printing process, the patterned substrate was cured in a temperature-controlled convection oven (SalvisLab Thermocenter) at 120° C. for 20 min. The substrate was finally cleaved to create single-use test strips possessing overall dimensions of 10 mm×34 mm. For the OS experiments, CoPC microparticles were dispersed in the carbon ink (2% w/w) due to the insolubility of the electrochemical mediator in the solution phase. For the LI experiments, a glassy carbon (GC) disc electrode was used as a working electrode.

Composition and Operation of Channels for the Analysis of Injuries:

Among pervasive battlefield injuries, soft tissue injury (STI), traumatic brain injury (TBI), acute liver injury (LI), abdominal trauma (ABT), hemorrhagic shock (HS), and oxidative stress (OS) are the most common sustained by soldiers in combat.¹⁹⁻²²

1. Soft Tissue Injury (STI): Gly-Gly buffer, 50 mM, with 6.7 mM MgAc was titrated by KOH to the pH value of 7.95 and used as a background solution (note that Mg²⁺ and K⁺ cations are essential for activation of CK and PK, respectively). The following components were dissolved in this solution to perform the NAND logic operation: NADH (0.3 mM), BSA (0.03% w/v), ATP (2 mM), PEP (0.5 mM), PK (2 U mL⁻¹), creatine (15 mM). Logical ‘0’ and ‘1’ levels of CK (0.1 and 0.71 U mL⁻¹) and LDH (0.15 and 1 U mL⁻¹) input signals were applied to the logic system in order to realize meaningful circulating levels of these biomarkers. Immediately following mixture, optical absorbance measurements were recorded continuously for 300 sec at λ=340 nm. The solutions for the electrochemical measurements were incubated for 180 sec at 37° C., then an MG redox mediator (0.3 mM) was added to catalyze electrochemical oxidation of NADH on the SPE, and a chronoamperogram was recorded at E=0.0 V.

2. Traumatic Brain Injury (TBI): Potassium phosphate buffer, 50 mM, pH 7.4, containing GlOx (1 mU mL⁻¹) and MP-11 (0.44 μM for optical and 5 μM for electrochemical measurements) was used to perform the AND logic operation. Logical ‘0’ and ‘1’ levels of NE (2.2 nM and 3.5 μM) and GLU (40 μM and 140 μM) were applied to the logic system in order to realize meaningful circulating levels of these biomarkers. The norepiquinone (NQ) produced in situ was optically measured at λ=487 nm. Chronoamperometric detection of NQ was performed on the SPE at E=−0.4 V.

3. Liver Injury (LI): Tris-HCl buffer, 100 mM, pH 7.4, was used as a background solution. L-ALA (200 mM), KTG, (10 mM) and NADH (136 μM) were dissolved in this solution to perform the NAND logic operation. Logical ‘0’ and ‘1’ levels of ALT (0.02 and 0.2 U mL⁻¹) and LDH (0.15 and 1 U mL⁻¹) input signals were applied to the logic system in order to realize meaningful circulating levels of these biomarkers. The output signal corresponding to the decreasing concentration of NADH was measured optically at λ=340 nm. The solutions for the electrochemical measurements were incubated for 180 sec at 37° C., then the MG redox mediator (0.3 mM) was added to catalyze the electrochemical oxidation of NADH on a glassy carbon electrode and a chronoamperogram was recorded at E=0.8 V.

4. Abdominal Trauma (ABT): Potassium phosphate buffer, 50 mM, (pH 7.15 for optical or pH 7.40 for electrochemical measurements), containing 0.2 mM MgCl₂, 0.01 mM CaCl₂, and NAD⁺ (10 mM for optical or 1 mM for electrochemical measurements) was used to perform the AND logic operation. Logical ‘0’ and ‘1’ levels of LDH (0.15 and 1.0 U mL⁻¹) and lactate (1.6 and 6.0 mM) input signals were applied to the logic system in order to realize meaningful circulating levels of these biomarkers. The output signal corresponding to the NADH formation was measured optically at λ=340 nm. The mixture for electrochemical measurements was incubated for 180 sec at 37° C., then the MG redox mediator (0.3 mM) was added to catalyze electrochemical oxidation of NADH and a chronoamperogram was recorded at E=0.1 V.

5. Hemorrhagic Shock (HS): Potassium phosphate buffer (50 mM, pH 7.4 for optical or 100 mM, pH 7.0 for electrochemical measurements) containing GOx (2 mU mL⁻¹) and MP-11 (0.44 μM for optical and 5 μM for the electrochemical measurements) was used to perform the AND logic operation. Logical ‘0’ and ‘1’ levels of NE (2.2 nM and 3.5 μM) and GLC (4 mM and 26 mM) were applied to the logic system in order to realize meaningful circulating levels of these biomarkers. The norepiquinone (NQ) produced in situ was measured optically at λ=487 nm. Chronoamperometric detection of NQ was performed on the SPE at E=−0.4 V.

6. Oxidative Stress (OS). Citrate buffer, 50 mM, pH 5.0 was used as a background solution. DTNB (2 mM) and NADPH (180 μM) were dissolved in this solution to perform the AND logic operation. Logical ‘0’ and ‘1’ levels of GSSG (150 μM and 400 μM) and GR (0.55 U mL⁻¹ and 0.65 U mL⁻¹) input signals were applied to the logic system in order to realize meaningful circulating levels of these biomarkers. Optical analysis of the reduced glutathione (GSH) produced in situ was performed according to the standard procedure using Ellman's reagent (DTNB).²³ Electrochemical analysis of GSH was performed by chronoamperometric measurements at E=0.5 V on the CoPC-modified SPE in a similar system without DTNB.

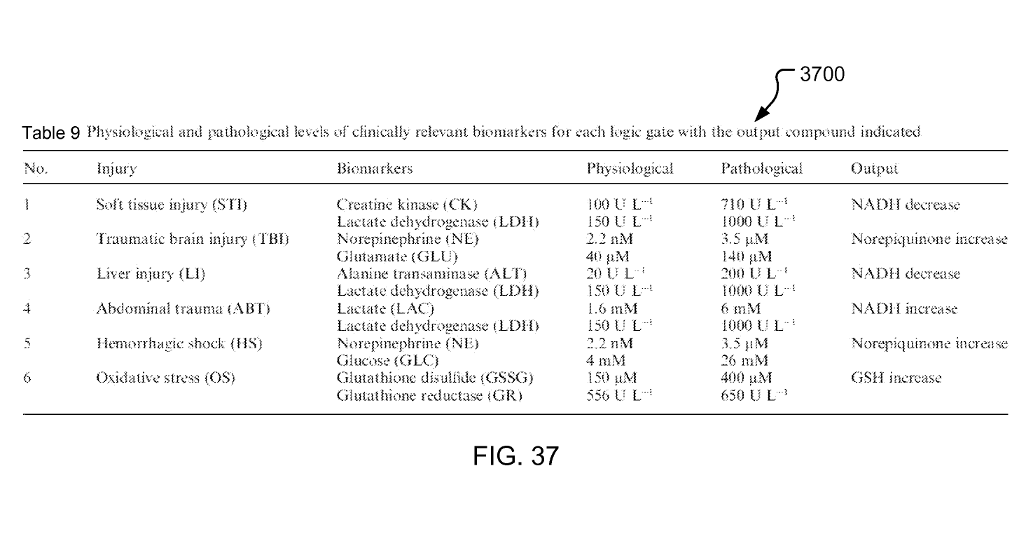

The concentrations of the biomarker input used for the activation of the logic gates are summarized in table 9 (3700) of FIG. 37.

A new modular biocomputing coding based on parallel and multiplexed enzyme logic gates has been described. This enzyme logic coding approach has been demonstrated towards a multi-injury diagnosis in connection to twelve biomarker inputs. This system is able to assess 64 individual pathological conditions among 4096 possible pathophysiological states through multiplexing the outputs into a 6-bit ‘injury code’. The new system includes AND and NAND gates and is able to evaluate a greater number of injuries than permitted by single enzyme logic gates while leveraging an electronic backbone of similar complexity. Moreover, due to the technique's Boolean biochemical signal processing architecture, the system is able to infer pathological conditions with a greater degree of reliability while enabling lower power consumption operation due to the reduced dependence on electronic operations than conventional biosensor arrays are currently able to offer. The technique represents the first demonstration of the parallelization of enzyme logic gates applied to diagnostic merits as well as the simultaneous multiplexing of the outputs of multiple logic gates in the biochemical domain into a binary injury code. Such devices can be used to implement low-cost, disposable biosensors. The injury code concept is well-positioned to enable the rapid and reliable assessment of multiple life-threatening injuries away from the hospital setting. Moreover, in addition to its connection to multi-injury diagnosis, the concept could be extended to the reliable assessment of a wide range of other practical medical, industrial, security and environmental scenarios.

Responsive Interface Switchable by Logically Processed Physiological Signals-Towards “Smart” Actuators for Signal Amplification and Drug Delivery

In another aspect, described are enzyme logic systems functioning as logic AND gate for processing biomarker characteristics of liver injury, alanine transaminase and lactate dehydrogenase. The NAD⁺ output signal produced by the system upon its activation in the presence of both biomarkers can be then biocatalytically converted to a decrease in pH. The acidic pH value biocatalytically produced by the system as a response to the biomarkers can trigger the restructuring of a polymer-modified electrode interface. This can allow a soluble redox species to approach the electrode surface, thus switching the electrochemical reaction ON. The redox transformations activated by the biochemical signals can result in an amplification of signals. This system can represent an integrated sensing-actuating chemical device with the implemented AND Boolean logic for processing natural biomarkers at their physiologically relevant concentrations.

This paper presents an example of a switchable electrode interface logically controlled by the enzyme cascade processing biomarkers characteristic of an injury. A liver injury, using the alanine transaminase (ALT) and lactate dehydrogenase (LDH) as the biomarker inputs for the AND logic gate, was selected to illustrate this concept. The biochemically controlled actuation achieved can be used to implement integrated “smart” ‘Sense/Act’ (biosensor-bioactuator) systems processing biomedical signals, making programmed decision, and then performing an actuation according to the biomedical needs.

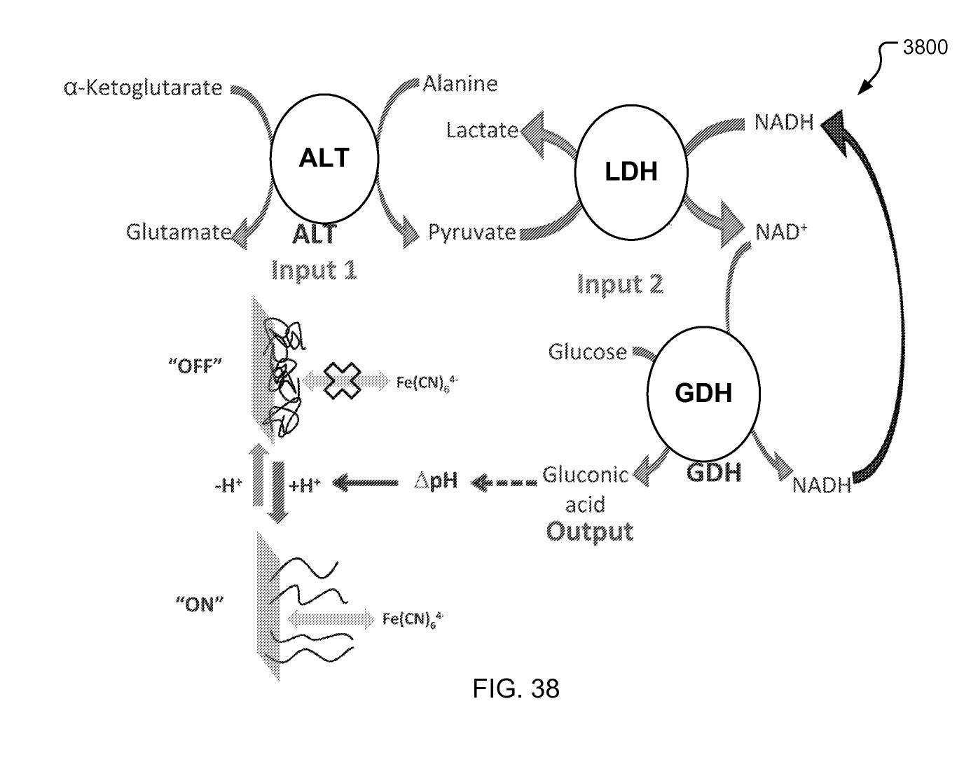

The enzymes ALT and LDH were selected as liver injury biomarkers (25). Their specificity for the injury is rather limited and elevated concentrations of each biomarker separately are not a solid evidence of liver injury. However, the appearance of both biomarkers at their characteristic elevated concentrations provides strong evidence of liver injury. A biocatalytic cascade which includes cooperative action of both biomarkers mimicking the AND Boolean logic gate was designed to signal the presence of these biomarkers (16). FIG. 38 shows the biocatalytic cascade (3800) activated by the biomarker inputs, ALT and LDH, primary resulting in the conversion of NADH to its oxidized form NAD⁺. The corresponding optical absorbance changes were used in an earlier work to analyze the performance of the AND logic gate upon application of different combinations of the input signals.

In order to convert the NAD⁺ signal to pH changes affecting the state of the modified electrode, GDH and glucose were added to the system to operate as the last step of the biocatalytic cascade. In situ generation of NAD⁺ resulted in the oxidation of glucose biocatalyzed by GDH, thus causing an acidification of the solution, and producing ΔpH as the final output signal. The biocatalytic cascade producing pH changes can be completed only in the presence of both the input signals, thus mimicking the AND logic operation. The logic 0 levels of the input signals correspond to the normal physiological concentrations of ALT and LDH, rather than to their complete absence. Thus, the input combinations 0,0; 0,1; and 1,0 also result in the reactions shown in FIG. 38, but yield substantially smaller pH changes than the combination 1,1, which corresponds to the pathophysiological elevated concentrations of the two biomarkers for liver injury. The challenge in the present work has been achieving the electrode activation only with the signal combination 1,1, while all other combinations preserve the OFF state of the electrode. In order to realize this specific switchable behavior of the P4VP-modified electrode, its pH-controlled transition from the OFF to ON state (23) must be coordinated with the pH output signals produced by the biocatalytic system.

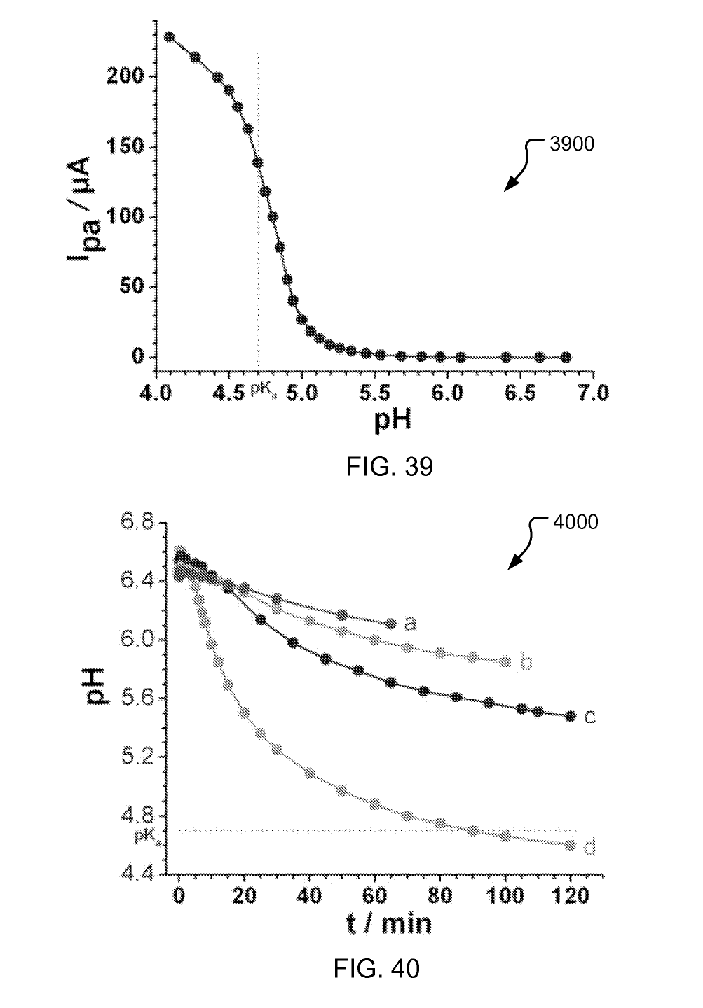

Interfacial properties of the P4VP-modified electrode are dependent on the protonation state of the polymer brush which is swollen and permeable for anionic redox species in its positively charged protonated state. The shrunken neutral state of the polymer brush is non-permeable for redox species, where the electrode is inactive. The reversible transition of the polymer brush between the charged and neutral state, corresponding to the ON and OFF states of the electrode, respectively, can be achieved by changing the pH value of the external solution. However, it might be inhibited by adsorption of proteins; the switchable behavior of the electrode interface requires verification for each specific composition of the solution. FIG. 39 shows the titration curve (3900) corresponding to the electrode activity derived by cyclic voltammetry at variable pH values in the solution containing all chemicals included in the sensing system except the input signals. The electrode activity is switched OFF at solution pH values higher than 5, while there is a sharp transition to the ON state of the electrode below this pH value. The pKₐ=4.7 of the polymer brush was derived from the experimental titration curve.

In the next experiment we applied the two input signals, ALT and LDH, in four different combinations: 0,0; 0,1; 1,0 and 1,1, to generate the pH changes in situ upon activation of the biocatalytic cascade shown in FIG. 38. FIG. 40 shows the time-dependent pH changes (4000) for the four input combinations. The important feature of the system is a large separation of the pH changes generated in the presence of 1,1 input signals from all other combinations. The pH changes produced in situ in the presence of 1,1 input signals reach the pKₐ value of the polymer brush at a reaction time of 90 minutes, while other signal combinations do not produce this pH value at any reaction time (measured up to 3 hours). Such distinct pH-time profiles can allow selective electrode activation in the presence of the 1,1 input combination characteristic of liver injury as shown in FIG. 38.

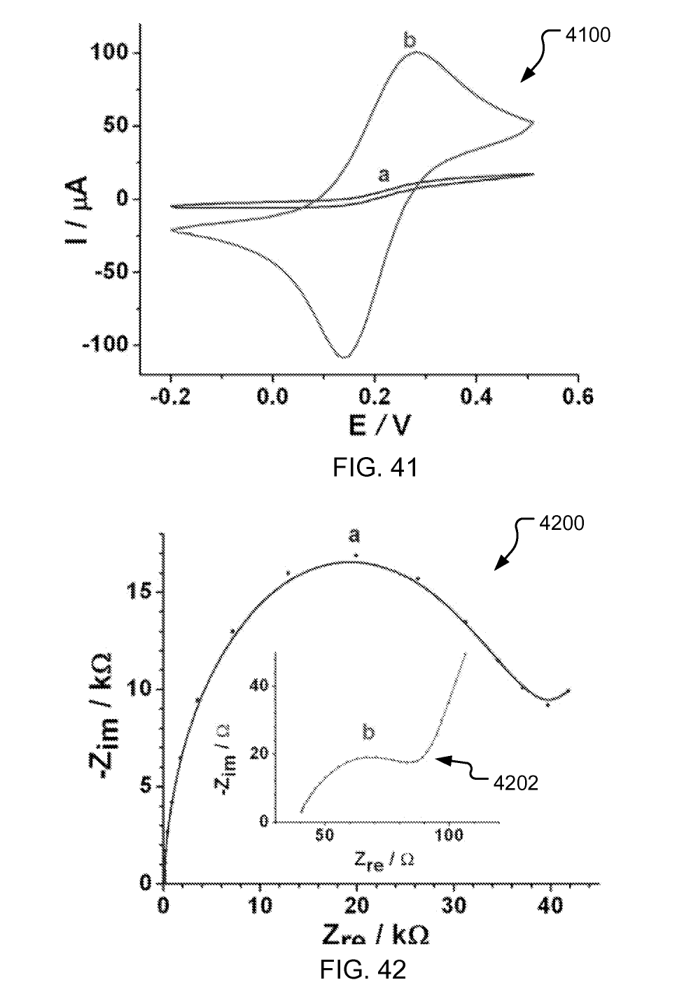

FIG. 41 shows cyclic voltammograms (4100) obtained with the P4VP-modified electrode being in the original OFF state at pH 6.3 (curve a) and after the electrode was activated by 1,1 input signals reaching the pH 4.75 generated in situ in 80 minutes (curve b). While the electrode being in the initial OFF state shows only very small activity for the soluble redox probe, the same electrode after its activation with 1,1 input signals shows a reversible cyclic voltammogram characteristic of the diffusional electrochemical process. All the other input combinations did not change the electrode state, even for much longer reaction times demonstrating the cyclic voltammograms similar to curve a in FIG. 41. Consistent results were obtained upon measuring the Faradaic impedance spectra with the switchable electrode. FIG. 42 (curve ‘a’ 4200) shows the impedance spectrum at the initial OFF state of the electrode with the electron transfer resistance, Rₑₜ, ca. 40 kΩ. Application of the 1,1 input combination resulted in the opening of the electrode interface and the decrease of the Rₑₜ to 60.3Ω (curve ‘b’ 4202). All other combinations of the input signals did not change the Rₑₜ value, demonstrating impedance spectra similar to the one shown in FIG. 42 (curve ‘a’ 4200).

The primary redox species changing the concentrations upon the operation of the AND logic gate, NADH/NAD⁺, appear at concentrations of 0.3 mM, while the redox species reacting at the switchable electrode, [Fe(CN)₆]⁴, has a much higher concentration of 10 mM. The primary biochemical species, processed through the biocatalytic cascade, trigger the opening of the electrode surface for the electrochemical reaction with a high concentration of the reacting species, thus resulting in the amplification of the output signal.

The possibility of chemical actuation triggered by appropriate combinations of medically relevant biomarkers applied under pathophysiologically elevated concentrations. A switchable polymer-modified electrode was selectively activated by the 1,1 input signals combination, which was processed by the enzyme logic system operating as the AND gate and transduced to pH changes controlling the interfacial properties of the electrode. Small concentration changes of the NADH/NAD⁺ system were converted into a large current corresponding to the electrochemical process of the K₄[Fe(CN)₆] redox probe, thus amplifying the output signal generated by the enzyme logic system. A switchable electrode controlled by biomarker inputs can be integrated into a biofuel cell (26) being activated upon pathophysiological conditions characteristic of a specific injury. The system can also be redesigned to open a channel in a microfluidic device for a drug release triggered upon receiving the output signal from the enzyme logic, thus opening the opportunities for the ‘Sense/Act’ (biosensor-bioactuator) integrated systems.

Enzymatic AND Logic Gates Operated Under Conditions Characteristic of Biomedical Applications

In another aspect, described are experimental and theoretical analyses of the lactate dehydrogenase and glutathione reductase based enzymatic AND logic gates in which the enzymes and their substrates serve as logic inputs. These two systems are examples of the novel, previously unexplored, class of biochemical logic gates that illustrate potential biomedical applications of biochemical logic. They are characterized by input concentrations at logic 0 and 1 states corresponding to normal and pathophysiological conditions. The logic gates described herein have similar noise characteristics. Both significantly amplify random noise present in inputs, however for realistic widths of the input noise distributions, it is still possible to differentiate between the logic 0 and 1 states of the output. This indicates that reliable detection of pathophysiological conditions is indeed possible with such enzyme-logic systems.

Described in this document are experimental and theoretical analyses of two kinetically similar AND logic gates activated by enzymes and their corresponding substrates. The first AND logic gate was activated by lactate dehydrogenase (LDH) and lactate—jointly constituting a definitive set of biomarkers for abdominal trauma (ABT), while the second AND logic gate was activated by glutathione reductase (GR) and glutathione disulfide (GSSG), which are indicative of oxidative stress when their concentrations are elevated.

The described biochemical digital computing systems can operate at specific levels of logic inputs determined by physiological conditions with 0 and 1 corresponding to “normal” and “abnormal” concentrations. The logic 0 and 1 input signals may accordingly have small range of variation, thus resulting in poor “digital” discrimination of the generated output signals. Furthermore, the output signal values interpreted as logic 0 and 1 can have significant variation as compared to the difference between the two reference logic points. Finally, functioning at physiological concentrations can impose strict constrains on the possible values or ranges of concentrations, and leave little margin for variation of the process parameters for optimizing the gate functioning. In order to consider the quality and possible optimization of the biochemical logic gates operation, the whole surface-response function should be evaluated and analyzed for variable concentrations of input signals.

Biochemical logic gates can use enzymes operating as the gate's “machinery,” which served to process signals represented by the corresponding substrates/cosubstrates. In some implementations, enzymes can be used as input signals to activate the “machinery-soup” composed of all the other reactants. The present biocomputing gates can be activated upon the simultaneous supply of an enzyme and a corresponding substrate, as inputs for the AND logic operation. This can pose additional challenges for modeling of system performance and from this point of view such gates can be regarded as examples of a novel, previously unexplored, class of biochemical logic systems. Since input signals in these gates correspond to physiological conditions, there can be some uncertainty present in their values, because chemical concentrations obviously vary from case to case, i.e., there is some distribution of input signals or, in other words, noise. The described systems are illustrative of a broad range of biomedical applications, analysis and understanding of how this noise in the input propagates in such logic gates.

Experimental Procedure for the Lactate/LDH and Gate:

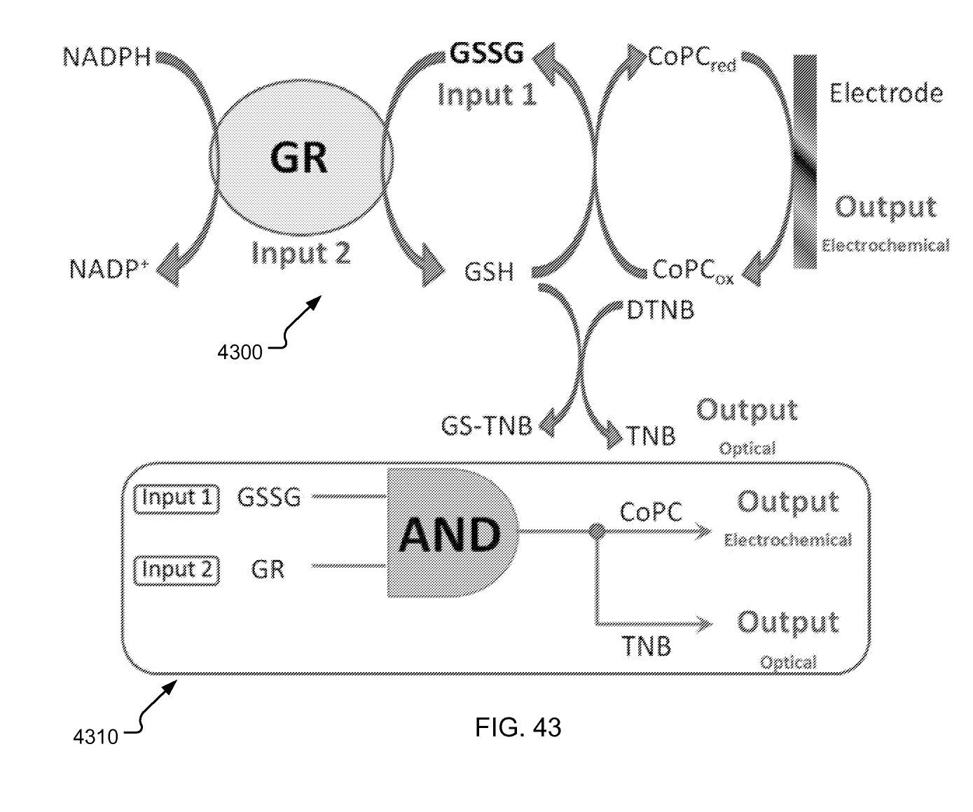

A graphical representation of an actate/LDH biocatalytic cascade (4300) and its AND logic gate (4310) equivalent are shown in FIG. 43. MGₒₓ and MG_{red} are the oxidized and reduced forms of MG, respectively. The enzymatic AND logic gate (4310) “machinery” can include NAD⁺ (10 mM and 1 mM for optical and electrochemical measurements, respectively) in 50 mM sodium/potassium phosphate buffer with 0.2 mM MgCl₂ and 0.01 mM CaCl₂, pH=7.15. MG, 1 mM, can be added as a mediator catalyzing NADH oxidation for the electrochemical measurements. The AND logic gate can be activated by lactate and LDH as Input 1 and Input 2, respectively. Logic 0 and 1 levels of lactate (1.6 and 6.0 mM) and LDH (150 and 1000 UL⁻¹—given in activity units per liter) input signals can be selected according to the mean normal and elevated physiological concentrations of these biomarkers relevant for the diagnosis of abdominal trauma. The optical measurements employed the transmission method (absorbance at λ=340 nm) and the electrochemical measurements were performed using the amperometric technique (continuous agitation and 0.1 V applied at the working electrode vs. an Ag/AgCl reference) in order to monitor the generation of the NADH product. In the optical experiments, the absorbance measurements were started immediately after mixing the reagents in a cuvette and the final absorbance was taken at 360 sec from the beginning of the measurements. In the electrochemical experiments, the buffer, lactate, LDH, and MG solutions were dispensed in the microcell and the amperometric recording initiated. Following a 150 sec settling period to allow the background current to decay to stable levels, the NAD⁺ solution was added and the recording continued for an additional 150 sec. At 300 sec following the initiation of the recording (150 sec following the addition of NAD⁺), the output-signal current reading was extracted. In order to remove the contribution of the background current to the bona-fide electrochemical signal arising from the biocatalytic process, the current obtained just prior to the addition of NAD⁺ at 150 sec was subtracted from the current reading at 300 sec. In order to map the response-surface of the AND gate, we varied lactate and LDH concentrations obtaining an array of 6×6 experimental points [concentration step in electrochemical (optical) measurements was 1.5 (1.2) mM, with variation between 0 (1) and 7.5 (7) mM for lactate, and step of 250 (200) UL⁻¹, with variation between 0 (100) and 1250 (1100) UL⁻¹ for LDH]. The varied concentrations of the input signals scanned the ranges somewhat below the logic 0 values to somewhat above the logic 1 values.

Experimental Procedure for the GSSG/GR AND Gate:

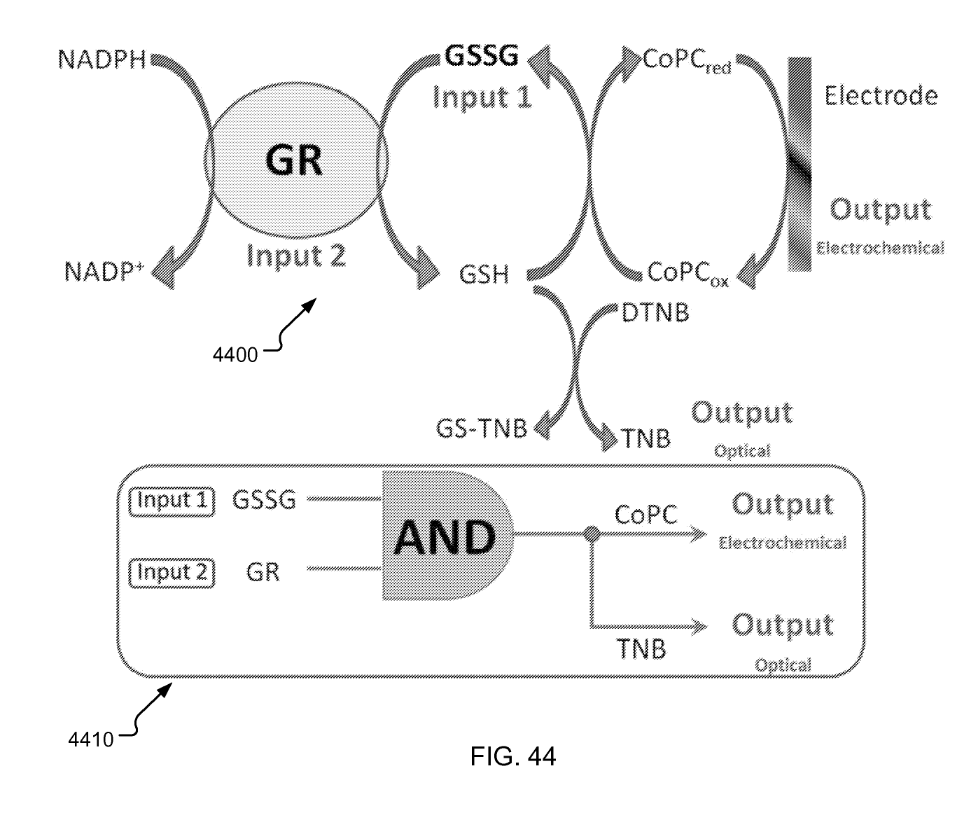

A graphical representation of a GSSG/GR biocatalytic cascade (4400) and its AND logic gate (4410) equivalent are shown in FIG. 44. CoPCₒₓ and CoPC_{red} are the oxidized and reduced forms of CoPC, respectively. The enzymatic AND logic gate (4410) “machinery” can include 0.18 mM NADPH (DTNB, 2 mM, was added for optical measurements) in 50 mM citrate buffer, pH=5.0. The AND logic gate was activated by GSSG and GR as Input 1 and Input 2, respectively. Logic 0 and 1 levels of GSSG (150 and 400 μM) and GR (556 and 650 UL⁻¹) input signals were chosen according to the mean normal and elevated physiological concentrations of these biomarkers in erythrocytes, motivated by studies of oxidative stress. Since GR and GSSG are mainly present in intracellular compartments of erythrocytes, maintaining a physiological pH (7.35-7.45 for serum) was not essential for the assay. Thus, in order to attenuate the enzymatic reaction and to provide output signals significantly different for each level of inputs, the pH was experimentally optimized and adjusted to the final value of 5. In the optical experiments the thiol groups of the biocatalytically produced GSH irreversibly reacted with DTNB resulting in the formation of thio-(2-nitrobenzoic acid), TNB, which was monitored by absorbance changes at λ=412 nm. The absorbance changes were measured during the reaction following the reactants mixing in a cuvette and the final absorbance value was taken at 100 sec from the beginning of the reaction. In the electrochemical experiments CoPC (2% w/w) was employed as a redox mediator to enable the low-potential detection of GSH at 0.5 V vs. Ag/AgCl reference. The buffer, GR, GSSG, and NADPH solutions were dispensed on the CoPC-modified SPE and the chronoamperometric recording (in a quiescent solution) initiated. Following a 30 sec settling period to allow the transient current to decay to negligible levels, the recording was terminated and the reading was extracted. In order to map the response-surface of the AND gate, we varied the input concentrations and obtained an array of 6×6 experimental points (concentration step of 83 μM between 67 and 483 μM for GSSG and concentration step of 31 UL⁻¹ between 524 and 679 UL⁻¹ for GR). Note that the varied concentrations of the input signals here also started below the logic-0 values, and then again exceeded the logic-1 values.

Model of the AND Gate Function

The initial concentrations of Input 1 and Input 2 chemicals vary between some minimum (not equal to zero), [Input 1] (t=0)=C_{1,min}, [Input 2] (t=0)=C_{2,min}, and maximum, [Input 1] (t=0)=C_{1,max}, [Input 2] (t=0)=C_{2,max}, values determined by specific biomedical applications. The output product concentration, P, is measured as P(t=t_{gate}) at specific reaction time, t_{gate}, and also varies between two values, Pₘᵢₙ and Pₘₐₓ, as the input concentrations are swept from their minimal to maximal values. To analyze the logic gates, input/output chemical signals can be cast in terms of the dimensionless input (x, y) and output z variables scaled to the “logic” ranges,

$$x = \frac{{C}_{1} - {C}_{1 , min}}{{C}_{1 , max} - {C}_{1 , min}} , \\ y = \frac{{C}_{2} - {C}_{2 , min}}{{C}_{2 , max} - {C}_{2 , min}} , \\ z = \frac{P - {P}_{min}}{{P}_{max} - {P}_{min}} . \qquad ( 1 )$$

The noise amplification properties of the AND gate can be conveniently analyzed by considering the function z(x, y), termed the response surface, in the vicinity of the logic points (x, y)=(0,0), (0,1), (1,0), (1,1). In general, the gate output depends not only on the initial inputs and the time of the reaction, but also on other system parameters such as, for example, in the present case the initial concentration of the cosubstrate, c,

P=F(C₁,C₂;c,pH, . . . ; t_{gate}, . . . ). (2)

These additional parameters are divided, which could be physical or (bio)chemical, in Equation (2) into two groups. Certain parameters, here exemplified by c and pH, can be adjusted to some extent to improve the gate performance. Other parameters cannot be easily adjusted, here, for instance, t_{gate}, which was chosen to have the best possible separation between the 1 and 0s of the output, as well as most physical properties such as the temperature, which are fixed by the intended application.

In order to calculate the function z(x, y), the parameter dependence in Equation (2) can be modeled. There are several sources of noise to be considered in biochemical computing. In addition to the natural fluctuations in the inputs about the precise logic-point values, there can also be systematic deviations, as well as uncertainty in the physiological concentrations which replace the sharp logic-value definitions with ranges. The output, similarly, is not precisely defined. In addition to the spread of their values, outputs can be somewhat shifted from the selected reference “logic” answers. Furthermore, the shifts in the outputs will generally be somewhat different at different logic inputs which are supposed to yield the same logic-value answers in the binary convention: here the three inputs (0,0), (0,1), (1,0), which are all expected to yield 0 output.

Thus, the functional dependence in Equation (2) might not be precisely defined, and should be viewed as an average with possible small systematic shifts from the “ideal” logic values built in. One way to evaluate the function F by fitting data from experiments, is to solve a set of precise or phenomenological kinetic equations corresponding to enzymatic reactions; this was the approach taken in previous works for several biochemical systems. Rate constants of the involved chemical reactions were then treated as adjustable parameters with values determined from the numerical fitting of the experimental data to the solution of the kinetic equations. This approach requires a working knowledge for the kinetics of the studied logic gate, which is not the case for the present systems due to their complexity. Indeed, even a simplified kinetic description would require fitting too many adjustable parameters which consequently cannot be accurately determined from the available gate-response mapping data.

Another approach has been to specify a phenomenological fit function for F, based on our experience with the solution of the kinetic equations as well as on the experimental expectations and observations. This approach has been developed for general analysis of the noise performance of logic gates and networks, when detailed information of the system's kinetics is either unavailable or the set of the reactions is far too complex to determine fitting parameters uniquely.

Indeed, we are only interested in the global features of the response surface function z(x, y), sufficient to evaluate its behavior in the vicinity of the four logic-point values. However, an earlier phenomenological fitting form developed for typical biochemical reactions encountered in AND-gate realizations with the logic 0s at zero concentrations, actually reflects the features of the biochemical reaction rather than more generally those of the binary logic. Therefore, it is inappropriate to use such a form for gates with non-zero logic-0 values. To avoid this complication, all the data were not rescaled upfront to the logic ranges, according to Equation (1). Instead, the idea is to fit the function F in Equation (2) first, by using the phenomenological shape-function, which here can be written as

$$F = \frac{φ {C}_{1} {C}_{2}}{( α {C}_{1} + 1 ) ( β {C}_{2} + 1 )} . \qquad ( 3 )$$

The resulting function can then be used to derive an estimate for z(x, y). The overall constant φ in Equation (3) may not be a useful fitting parameter because this factor will cancel in the calculation of z(x, y). Therefore, the actual data fits, described below, were done for F normalized to (divided by) its measured value at input concentrations at which it was the largest, and this ratio was least-squares-fitted in order to determine the two fitting constants of interest, α and β.

Because of the non-zero values of the minimum input concentrations, the output concentration possesses somewhat different values for the combinations of the inputs (C_{1,min}, C_{2,min}), (C_{1,min}, C_{2,max}), (C_{1,max}, C_{2,min}). In other words, output at logic 0 has three hopefully close but different values. However, for a “standard” AND gate, the logic-0 output should be equal for all these inputs. This implies that enzymatic gates considered in this work have some a systematic “noise” built-in. This “noise” exists in addition to the usual, random noise which was extensively analyzed earlier. Thus, the demands for additional network elements with filtering properties, will be even more stringent here if the present systems are to be used as components of biochemical logic networks. The present modeling approach is also somewhat different from earlier variants in that the mean values to be used (defined) as Pₘᵢₙ and Pₘₐₓ are not known upfront. Rather, we first evaluate the function in Equation (2) and then calculate Pₘₐₓ and the smallest logic 0 value as Pₘᵢₙ (another option would be, e.g., an average of the three logic 0 outputs as Pₘᵢₙ). Indeed, the output signals to be used as reference 0 and 1, are not arbitrary but are established by the application, up to the aforementioned built-in noise, which should be small to have a high-quality realization of the AND function.

If the phenomenological fit of Equation (3) is a good one, then α(c, pH, . . . ; t_{gate}, . . . ) and β(c, pH, . . . ; t_{gate}, . . . ) should be functions of the chemical and physical parameters introduced in Equation (2). The present approach, however, does not attempt to obtain this kinetic information, and therefore offers no quantitative information on the dependence of the response surface function z on such parameters (in addition to its arguments x and y). Thus the present approach cannot be used to directly optimize gate functionality. However, qualitative arguments can usually be utilized to decide whether to increase or decrease of the overall gate activity to improve its performance. These usually require large changes in the “gate machinery” properties because the response surface is obtained in terms of the scaled variables, Equation (1), and is not sensitive to the leading order linear-response-type changes. For the present systems, with the chemical concentrations established at physiological conditions, large changes are not easy to achieve, and therefore the gate performance per se may not be readily optimized by varying the few experimental variables under our control. Instead, the degree of “noisiness” of the realized gates' operation can be estimated, in expectation that networking with other elements (reaction steps) such as filters, when developed, will be a proper approach to obtain systems with less noise.

To analyze random noise amplification from input to output, we calculate the noise amplification factors as the ratio of the output noise distribution spreads, σᵢⱼ^{out}, to the fixed input noise distribution width, σⁱⁿ, with the definition

σᵢⱼ^{out}=[ [symbol] z² [symbol] ᵢⱼ− [symbol] z [symbol] ᵢⱼ²]^{1/2}, (4)

where the averages [symbol] . . . [symbol] at each logic point, (i, j)=(0,0), (0,1), (1,0), (1,1), are computed with respect to the input noise distribution Dᵢⱼ(x, y)=Xᵢ(x)Yⱼ(y), which for simplicity is assumed to be uncorrelated: a product of Gaussians with equal width, σⁱⁿ, in terms of the scaled variables. We used the most straightforward expression,

[symbol]

zⁿ [symbol] ᵢⱼ=∫∫zⁿ(x,y)Dᵢⱼ(x,y)dxdy, (5)

for numerical computations. For the description of the effect of the systematic noise—the built-in shift which translates into imprecise average values for z—we first compute the averages, [symbol] z [symbol] ᵢⱼ, and then the spread interval ( [symbol] z [symbol] ᵢⱼ−σᵢⱼ^{out}, [symbol] z [symbol] ᵢⱼ+σᵢⱼ^{out}) which defines the region where the output corresponding to the specific logic point is most likely to be found for the assumed distribution of random noise at the input. If the combined spread region for the three logic-0 points overlaps with or is too close to that for the logic-1 point, then the present gates cannot be used for systems with the degree of random noise in the input at the level of the σⁱⁿ value considered.

Lactate/LDH AND Gate

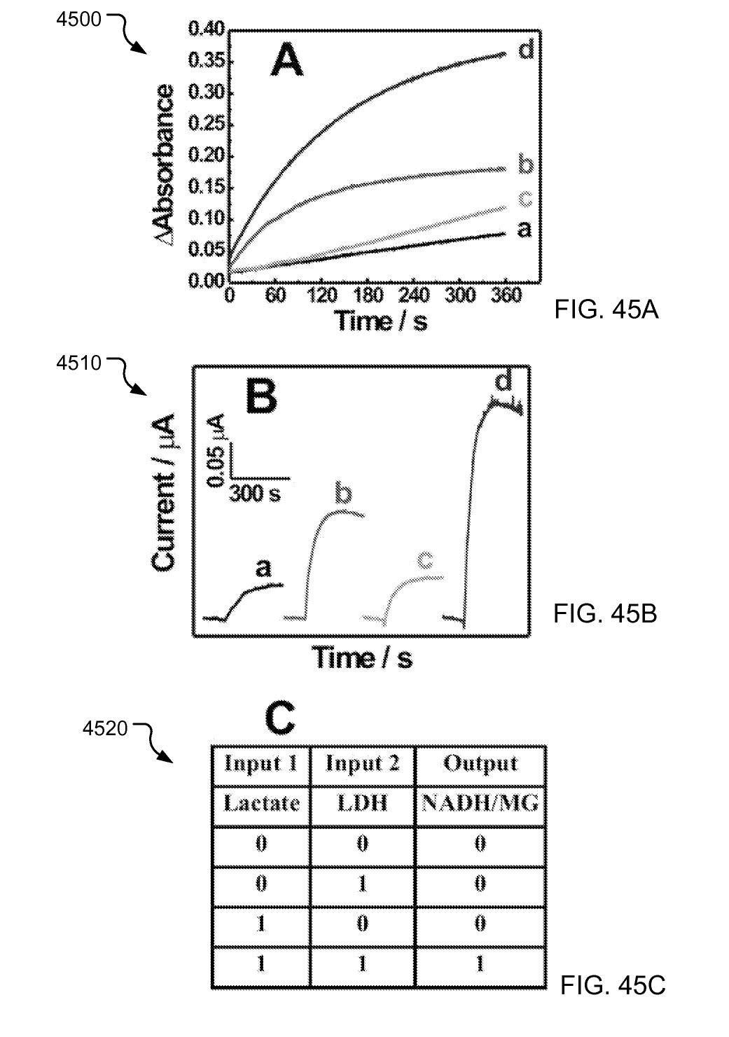

Catalytic reduction of NAD⁺ to NADH proceeding in the presence of lactate (Input 1) and LDH (Input 2) results in the increased concentration of NADH in the solution which can be followed by optical and electrochemical means as shown in FIG. 43. FIG. 45(A) shows exemplary optical absorbance (λ=340 nm) (4500). FIG. 45(B) shows exemplary electrochemical amperometric (0.1 V) detection of the NADH output generated in situ by the lactate/LDH biocatalytic cascade (4510) upon different combinations of the input signals: a) 0,0; b) 0,1; c) 1,0 and d) 1,1. The logic gate composition and the input concentrations corresponding to the logic 0 and 1 values are specified below. FIG. 45(C) is a truth table (4520) corresponding to the AND logic function of the system.

In the optical measurements, the absorbance increase characteristic of NADH formation (4500) was monitored at λ=340 nm (the system's background absorbance was subtracted) as shown in FIG. 45A. In the electrochemical experiments, the NADH oxidation was mediated by MG, and the obtained current values corresponded to the concentration of NADH produced in the course of the biocatalytic reaction (4510) as shown in FIG. 45B. The system represented AND logic gate when the reaction results in the product formation only in the presence of the both reacting species: the substrate (lactate) and the enzyme (LDH) [input signal combination (1,1)] as shown in the truth table (4520) as shown in FIG. 45C. However, since the logic 0 values of both input signals do not correspond to zero concentrations of the reactants, the reaction product is also generated at 0 logic values of one or both inputs [input combinations (0,0), (0,1), (1,0)]. In order to analyze the output function of the biocatalytic system for the given logic values of the input signals we performed the measurements with several varied concentrations of the substrate (lactate) and the enzyme (LDH).

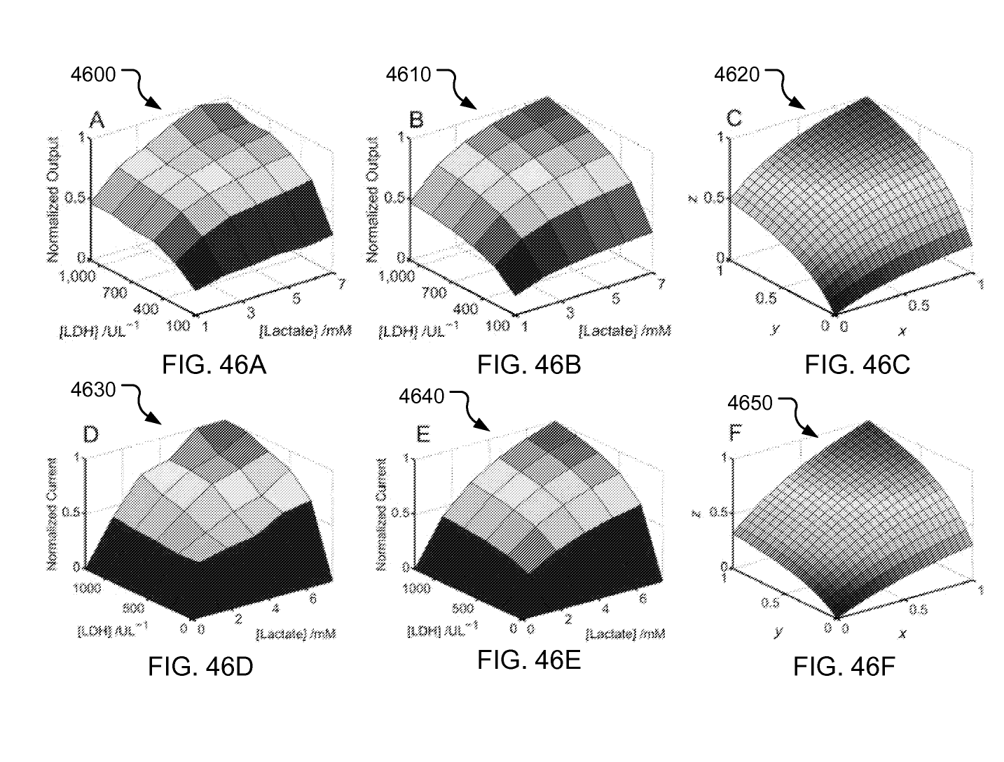

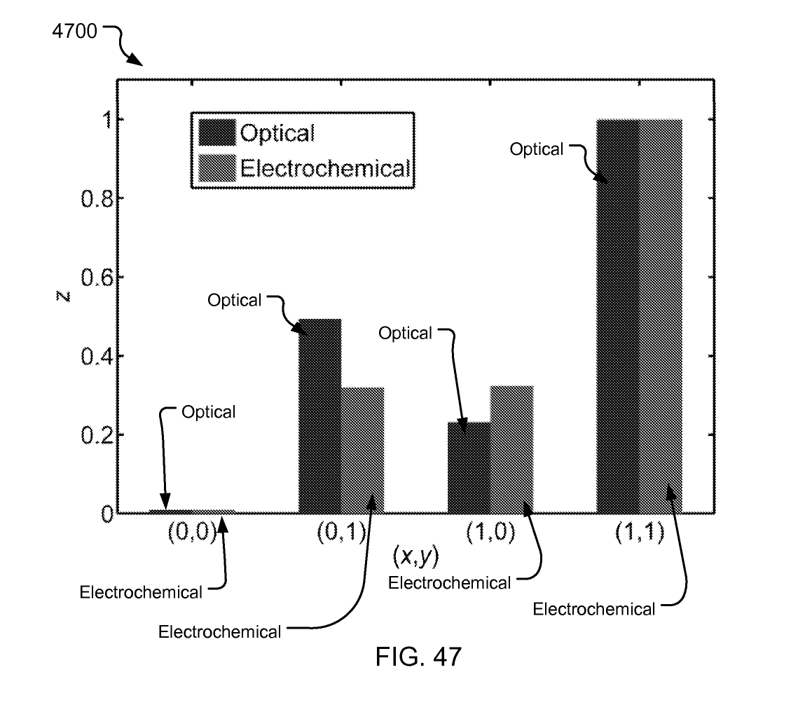

The normalized (to the maximum observed value) experimental response surface obtained by optical absorbance spectroscopy (4600, 4610 and 4620) is shown in FIGS. 46A, 46B and 46C for a reaction time t_{gate}=360 sec. The electrochemical current measurements (4630, 4640 and 4650) are shown in FIGS. 46D, 46E and 46F for a reaction time t_{gate}=300 sec. FIGS. 46A and 46D show the experimental response surface for the LDH-based AND gate. FIGS. 46B and 46E give the numerical fit according to Equation (3), with the resulting parameters a and 13 found to be 0.645 (0.241) (mM)⁻¹ and 3.68·10⁻³ (4.10·10⁻³) U⁻¹ L, for optical (electrochemical) sets of data, respectively. FIGS. 46C and 46F show the response surface in terms of the logic-range variables x, y, z, properly scaled and shifted (since the logic 0 values are not defined at zero concentrations).

As one can see, both the measured, FIGS. 46A and 46D, and the fitted, FIGS. 46B and 46E, surfaces obtained by the two different methods are quite consistent. The logic surfaces extracted from optical and electrochemical measurements, shown FIGS. 46C and 46F, are very similar to one another. This is also confirmed by comparing the output logic values at four logic points from the two data sets (4700) as shown in FIG. 4700. In this plot, the z-values corresponding to (0,0) and (1,1) logic input combinations are at zero and one, respectively, while z-values for two other logic input pairs, (0,1) and (1,0), fall in between. The outputs from the latter pair are not (close to) zero as they should be for a true AND gate because, as already mentioned, they correspond to different nonzero concentrations of input chemicals and, as such, cannot be made equal to each other or to the output at (0,0).

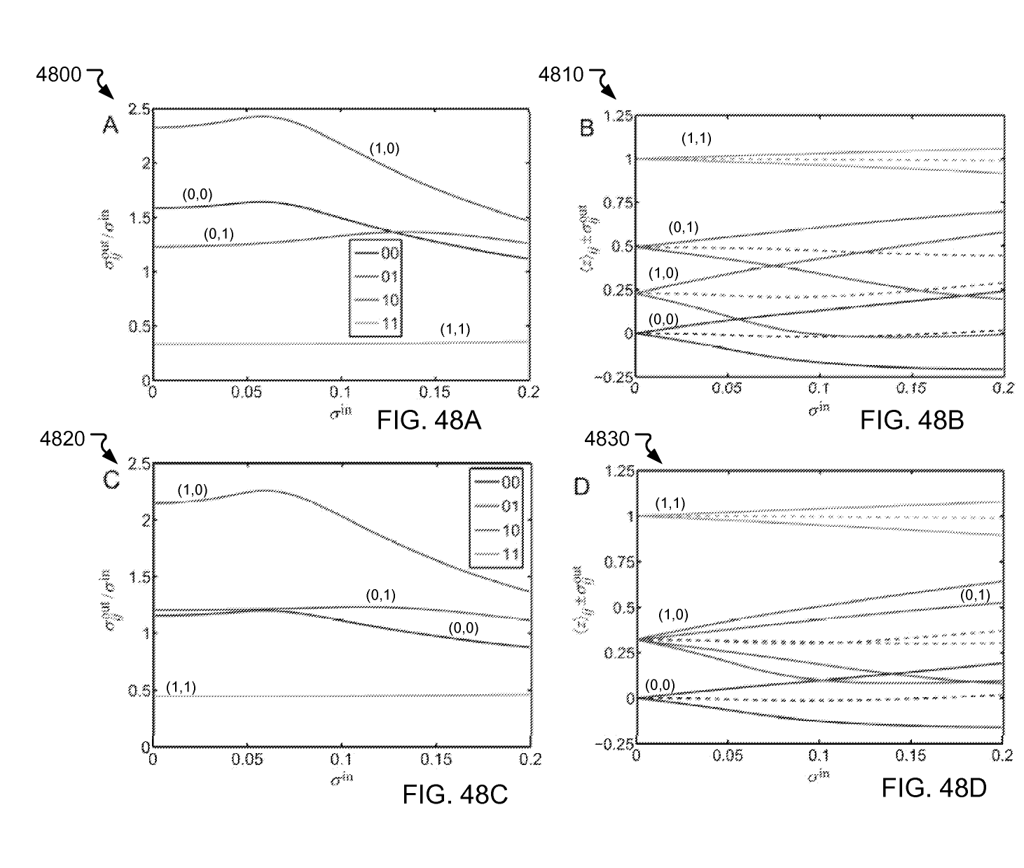

FIGS. 48A and 48B show noise propagation properties of the LDH-based AND gate, as calculated from fits of exemplary optical data. FIGS. 48C and 48D show noise propagation properties of the LDH-based AND gate, as calculated from fits of exemplary electrochemical data. FIGS. 48A and 48C show noise amplification factors σᵢⱼ^{out}/σⁱⁿ vs. the assumed width of the input noise distributions, σⁱⁿ. FIGS. 48B and 48D show the spread region vs. width of the input noise distribution σⁱⁿ. The dashed line is for the average value, [symbol] z [symbol] ᵢⱼ, of the logic output, while the solid lines of the same color give the upper and lower bounds of the spread region, [symbol] z [symbol] ᵢⱼ±σᵢⱼ^{out}.

The similarity between the two response surfaces is also manifested in similar noise characteristics evaluated for each response. Both the noise amplification factors, (4800 and 4820) illustrated in FIGS. 48A and 48C, i.e., the degree to which the random input noise is amplified at a particular logic point, and the spread regions (4810 and 4830), shown in FIGS. 48B and 48D, that provide information on the separation between the logic points for a given value of the input noise distribution width, agree both qualitatively and quantitatively. From the plots of σᵢⱼ^{out}/σⁱⁿ, we can gauge the response of the AND gate to noise in its inputs: If the maximum of σᵢⱼ^{out}/σⁱⁿ is larger/smaller than one, then the logic gate amplifies/suppresses incoming random noise. In particular, one can see that the “worst” (most noisy) logic point irrespective of the input noise spread, that with the largest ratio σᵢⱼ^{out}/σⁱⁿ, (1,0), at which the lactate/LDH concentration is the largest/smallest. Even though the noise is actually suppressed at the (1,1) logic point (Υᵢⱼ^{out}/σⁱⁿ≈0.5<1), poor performance (σᵢⱼ^{out}/σⁱⁿ>2, see FIGS. 48A-D) at the (1,0) logic point makes this system in general not very suitable for incorporation in large networks of biochemical logic gates without additional filtering elements aimed at reducing noise.

The described techniques of estimating the random noise amplification factor by assuming a Gaussian input distribution, becomes inappropriate for σⁱⁿ approximately exceeding 0.2, when the spread of the distribution becomes comparable to a sizable fraction of the unit interval. While the σᵢⱼ^{out}/σⁱⁿ curves tend to decrease for σⁱⁿ beyond ˜0.1, the overall shape of z(x, y) in the described systems is smooth-convex, which means that the AND gate always amplifies analog noise.

In practice, the physiological spread of the input concentrations is much less than 20%, at least for the (0,0) logic point. Indeed, the observed concentrations for lactate are within the 0.5-2.25 mM range, while the LDH values are spread over the 42-180 U/L interval. The average values that we employ as the input logic 0s are 1.6 mM and 150 U/L, respectively. This indicates that the distribution of the inputs is not symmetrical, and that the distribution spread is different for lactate and LDH. Because of this, the calculations only yield a qualitative estimate of the noise amplification at this logic point: By taking σⁱⁿ=10%, which is the average of the data spreads for the x and y logic inputs (˜15% and ˜5%, respectively), it can be deduced from FIGS. 48A and 48C, that the noise will be amplified by the factor of ˜120% at this logic point. Assuming that the input noise spread stays the same for all other logic points, it can be seen that maximum noise amplification produced by this gate would be ˜200%, at the (1,0) point.

This degree of amplification of the incoming noise does not, however, preclude practical utilization of the gate. Analysis of the spread region plots 4810 and 4830 in FIGS. 48B and 48D, shows that at σⁱⁿ=10%, the (1,1) logic point is well separated from other logic points by a “gap” of about 0.5, even though average outputs at (0,1) and (1,0) are not zero. However, if σⁱⁿ increases to ˜30%, the spread regions of (1,1) and (1,0) points begin to overlap, and it may not be possible to distinguish between the 0 and 1 levels of the output signal.

GSSG/GR AND Gate

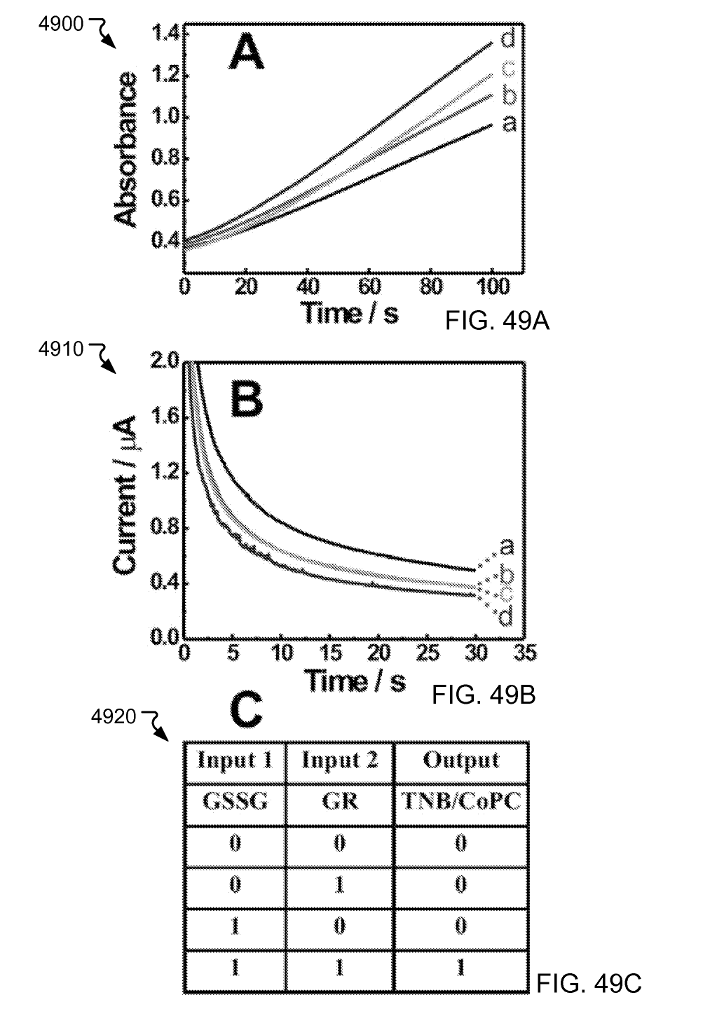

FIG. 49(A) exemplary optical absorbance (λ=412 nm) (4900), and FIG. 49(B) shows exemplary electrochemical chronoamperometric (0.5 V) detection of the GSH output generated in situ by the GSSG/GR biocatalytic cascade upon different combinations of the input signals (4910): a) 0,0; b) 0,1; c) 1,0 and d) 1,1. The logic gate composition and the input concentrations corresponding to the logic 0 and 1 values are specified in the Experimental Section. FIG. 49(C) is an exemplary truth table (4920) corresponding to the AND logic operation of the system.

The catalytic reduction of GSSG (Input 1) to GSH by GR (Input 2), that proceeds in the presence of NADPH (a part of the gate “machinery”), results in an elevated concentration of GSH in the solution which can be monitored by optical and electrochemical means as shown in FIG. 44. For optical measurements, in order to convert GSH to a chromogenic product, DTNB (Ellman's reagent) was employed which resulted the formation of thio-(2-nitrobenzoic acid), TNB—monitored optically at λ=412 nm (4900) as shown in FIG. 49A. For electrochemical experiments, the GSH oxidation was catalyzed by CoPC impregnated at an SPE. The current obtained (4910) corresponded to the concentration of GSH produced in the course of the biocatalytic reaction, FIG. 49B. The system represents an AND logic gate when the reaction results in the formation a product only in the presence of both reacting species: the substrate (GSSG) and the enzyme (GR)—input signal combination (1,1) as shown in the truth table (4920) of FIG. 49C. However, analogous to the lactate/LDH system, logic 0 values of the input signals do not correspond to zero concentrations of the reactants, so that some amount of the reaction product is also generated at logic 0 values of one or both inputs. In order to analyze the output function of the biocatalytic system for the given logic values of the input signals, we performed the measurements for the variable concentrations of both logic inputs: the substrate (GSSG) and enzyme (GR). Unlike the lactate/LDH system, which can serve directly as a logic gate injury detecting system for abdominal trauma, GR/GSSG is an example of a system which shows potential for intracellular investigations. Concentrations of the enzyme (GR) and its substrate (GSSG) used in the present study correspond to those inside erythrocytes. However, the presence of these biomarkers in blood is obvious when erythrocytes are ruptured, which can be symptom of radiation exposure or severe oxidative stress, thus further biomedical studies are needed to know the normal and pathophysiological concentrations of the biomarkers in blood.

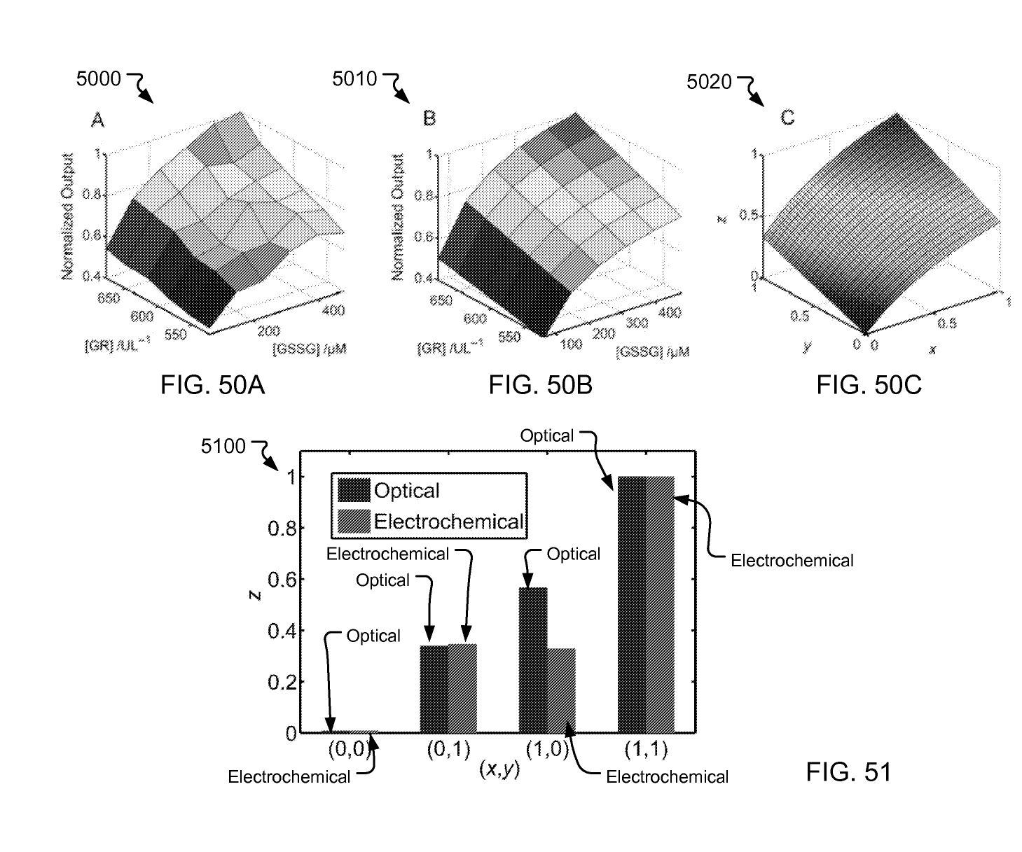

In general, the optically measured response surface and noise properties of this logic system were very similar to the one discussed above. One can see from the response surfaces (5000, 5010 and 5020) shown in FIGS. 50A, 50B and 50C and normalized logic outputs 5100 in FIG. 51 that the (0,1) and (1,0) points are displaced from zero, in direct correspondence with the results shown in FIGS. 45A-C and 46A-F for the other system. Also, the agreement between optical and electrochemical measurements at the logic points is satisfactory, as evident from FIG. 51. Therefore, for the GR/GSSG system electrochemical measurements were performed only at the four logic points rather than mapping the entire response surface.

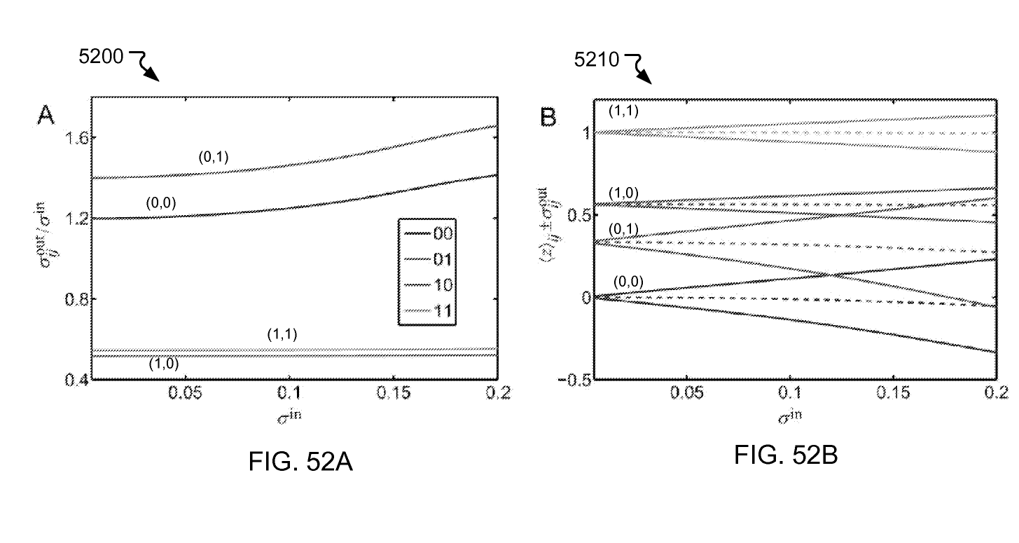

FIGS. 52A and 52B show noise propagation properties of the GR-based AND gate, as calculated from fit of the optical data. FIG. 52A shows noise amplification factors σᵢⱼ^{out}/σⁱⁿ vs. the assumed width of the input noise distributions, σⁱⁿ. FIG. 52B shows the spread region vs. width of the input noise distribution σⁱⁿ. The dashed line is for the average value of the logic output [symbol] z [symbol] ᵢⱼ, while the solid lines of the same color give the upper and lower bounds of the spread region, [symbol] z [symbol] ᵢⱼ±σᵢⱼ^{out}. The maximum noise amplification factor here is also larger than 100% (5200) as shown in FIG. 52A, which is not surprising given the overall convex shape of the response surfaces 5000, 5010 and 5020 in FIGS. 50A-C. The computed ratios σᵢⱼ^{out}/σⁱⁿ are actually smaller than those calculated for the LDH system (see FIGS. 48A-D). This indicates that the system exhibits a somewhat better noise performance, that is, it amplifies input noise to a lesser extent that the lactate/LDH logic gate (the maximum amplification factor here is 1.7 vs. 2.3 for the LDH based gate). However, because of the larger variation in the average z-values at the logic 0 points (see FIG. 51), the spread-region gap separating the logic 1 from logic 0 outputs (5210) is actually smaller (˜0.3, vs. ˜0.5 for the lactate/LDH gate), as seen in FIG. 52B. This is because of a very small physiological range of GR concentration that effectively brings 0 and 1 values of the Input 2 very close together.

Chemicals and Materials

The enzymes and other chemicals were obtained from Sigma-Aldrich and were used without further purification: glutathione reductase (GR, from S. cerevisiae, E.C. 1.6.4.2), lactate dehydrogenase (LDH, from porcine heart, E.C. 1.1.1.27), β-nicotinamide adenine dinucleotide dipotassium salt (NAD⁺), β-nicotinamide adenine dinucleotide 2′-phosphate reduced tetrasodium salt (NADPH), L(+)-lactic acid, L-glutathione disulfide (GSSG), cobalt(II) phthalocyanine (CoPC), methylene green (MG), dithiobis-(2-nitrobenzoic acid) (DTNB—Ellman's reagent) and other standard inorganic salts/reagents. Ultrapure water (18.2 MΩ cm) from NANOpure Diamond (Barnstead) source was used in all of the experiments.

Instrumentation and Measurements

A CH Instruments model 1232A potentiostat was used for all the electrochemical measurements and a Shimadzu UV-2450 UV-Vis spectrophotometer (with a TCC-240A temperature-controlled cuvette holder and 1 mL PMMA cuvettes) was used for all the optical measurements. A Mettler Toledo SevenEasy s20 pH-meter was employed for pH measurements. A Barnstead Thermodyne Cimarec stir/heat plate was employed to continuously agitate solutions and maintain temperature at 37° C. while electrochemical measurements were performed. All optical measurements were performed in temperature-controlled cuvettes/cells at 37° C. mimicking physiological conditions and all reagents were incubated at this temperature prior to experimentation. All electrochemical measurements were performed on a screen-printed electrode (SPE) and the potentials were measured vs. a quasi-reference Ag/AgCl electrode.

Screen-Printed Electrode (SPE) Preparation:

A screen printed three-electrode strip, custom-designed using AutoCAD®, consisted of a circular carbon working electrode (geometrical area: 3 mm²) inscribed in hemispherical counter (area: 10 mm²), and reference electrodes (area: 2 mm²). The fabrication of the flexible screen-printed electrode system is detailed as follows: An Ag/AgCl-based ink from Ercon (E2414) was employed to define the conductive underlayer as well as the reference electrode. A carbon-based ink (Ercon E3449) was subsequently overlaid on the conductive underlayer to define the counter and working electrode geometries. In the experiments with GR/GSSG, CoPC was dispersed in the carbon ink (2% w/w). Finally, an insulator ink (Ercon E6165) was overlaid on the Ag/AgCl and carbon layers to insulate all except the contact pads and the upper active segment of the electrodes. A Speedline Technologies MPM-SPM screen printer was used to print the pattern onto a 250 μm-thick flexible polyethylene terephthalate substrate (DuPont Melinex 329). Subsequent to the printing process, the patterned substrate was cured in a temperature-controlled convection oven (SalvisLab Thermocenter) at 120° C. for 20 min. The substrate was finally cleaved to create single-use test strips possessing overall dimensions of 10 mm×34 mm.

Various examples of experimental and theoretical analysis have been described of two AND logic gates activated by enzymes and their corresponding substrates. The first AND logic gate was activated by lactate dehydrogenase (LDH) and lactate, while the second AND logic gate was activated by glutathione reductase (GR) and glutathione disulfide (GSSG). Logic 0 and 1 levels of input signals were chosen according to the normal and pathological concentrations of these biomarkers relevant to the diagnosis of abdominal trauma for the lactate/LDH system and oxidative stress for GSSG/GR system. In order to analyze the output function of the biocatalytic systems for the given logic values of the input signals, we performed measurements with variable concentrations of both inputs in these enzymatic systems. The output product was detected by optical and electrochemical means that gave close results when cast in terms of the logic variables.

From numerical analysis of the response surfaces, both logic gates possess similar noise characteristics, i.e., they significantly amplify random noise in inputs with a maximum noise amplification factor of 2.3 and 1.7 for the lactate/LDH and GSSG/GR gates, respectively. Both of them also exhibit systematic noise due to the non-zero product output at logic 0. However, at realistic values of the input noise distribution widths, of the order of ˜10%, it is nevertheless possible to distinguish the logic-1 from logic-0 outputs even though the three logic-0 points (0,1), (1,0), (0,0) have substantial non-zero logic output values. This indicates that a reliable detection of pathophysiological conditions can be enabled by such logic gates. For larger spreads of the input noise or in a network of connected gates, noise suppression mechanisms, such as filtering, leading to a sigmoidal dependence of the output product on the chemical inputs, should be developed and utilized to allow for more complex information processing with biochemical logic.

Alert-Type Biological Dosimeter Based on Enzyme Logic System

In another aspect, described is an enzyme logic system that uses the cooperative effect of two biomarkers, α-amylase and lactate dehydrogenase, to analyze radiation-caused tissue damage in vitro in model solutions of human serum. The analytical system is based on the recently emerged biocomputing concept applying biocatalytic cascades for logic processing of biochemical input signals. The described systems resemble a Boolean NAND logic gate in which the change of the optical output signal from a high level (logic value 1) to a low level (logic value 0) confirmed the presence of both biomarkers at pathological concentrations (1,1 input signals), thus yielding the conclusion about a radiation tissue damage. The described systems can operate in a digital YES/NO format as an alert-type biosensor with a built-in Boolean logic.

Radiation injury, a life threatening form of organ tissue damage, is caused by exposure to ionizing radiation. A short intensive exposure results in acute radiation syndrome, while chronic radiation syndrome is caused by a prolonged exposure to lower intensity radiation. Immediate and effective medical treatment of persons suspected of radiation exposure requires a reliable detection approach to provide adequate diagnostic information. Biological dosimetry based on the analysis of biomarkers in a body exposed to radiation, rather than physical measure of a radiation dose, can be used as a tool for the effective medical management of radiation therapy for cancer, occupational radiation exposure (including astronauts in manned space missions), accidents similar to Chemobyl disaster, and possible radiological terror attacks. Radiation can result in different physiological processes, many of which are not specifically related to the direct instantaneous damage caused by radiation. Potentially important radiation injury biomarkers include various metabolites, proteins, RNA and DNA.

Techniques, apparatus and systems are described for implementing an enzyme-logic system for the analysis of biomarkers corresponding to the pathophysiological changes caused by radiation. Increased levels of α-amylase and lactate dehydrogenase can activate a biocatalytic cascade, resulting in the unambiguous conclusion on radiation-induced physiological processes.

Biodosimetry System

Among many known biomarkers characteristic of radiation-caused damage, selected are enzyme inputs able to operate in a biocatalytic cascade performing a logic operation based on the recently developed enzyme logic concept. An increased concentration of α-amylase (Amy) in blood, urine and saliva can result from irradiation exposure. Amy release originates mainly from gland tissue damaged by radiation. Another imminent effect of irradiation is tissue breakdown and hemolysis causing massive release of lactate dehydrogenase (LDH) into blood stream. Since Amy and LDH can be combined in a biocatalytic cascade resulting in consumption of NADH, these two enzymes can be selected as biomarkers of the radiation damage.

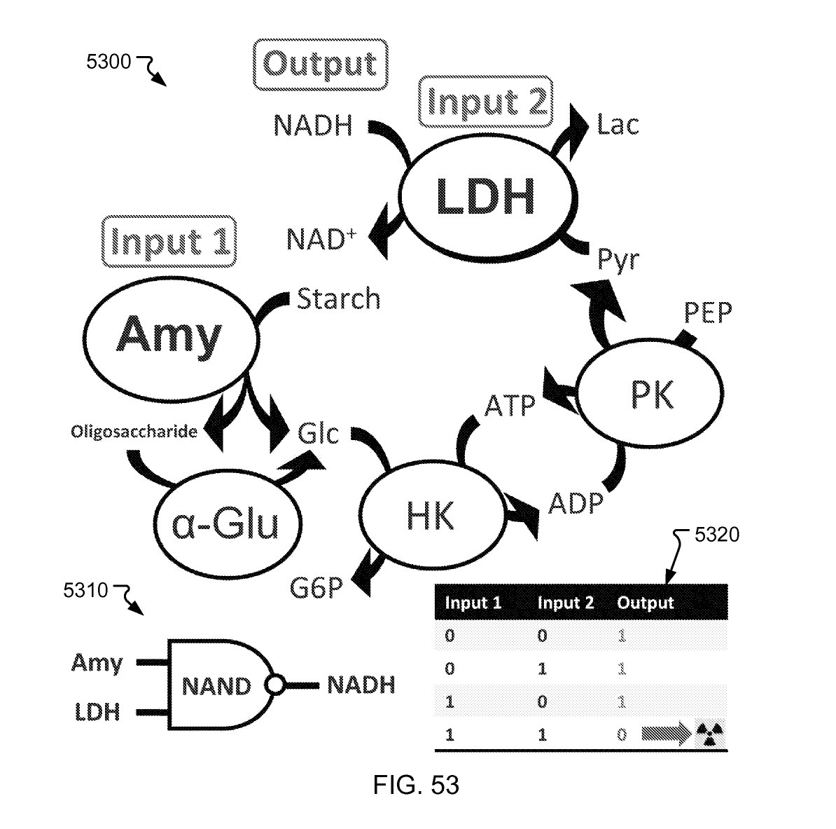

FIG. 53 shows an exemplary multi-enzyme biocatalytic cascade 5300 for the analysis of irradiation damage. Biomarkers Amy and LDH are applied as the input signals. NADH is the output signal. Other products of the biocatalytic cascade are the following: glucose (Glc), glucose-6-phosphate (G6P), pyruvate (Pyr) and lactate (Lac). For simplicity, the cascade does not include some reacting cofactors, promoters and byproducts. Schematic representation of the NAND logic gate (5310) and a corresponding truth table (5320) are shown at the bottom.

The biocatalytic cascade as shown in FIG. 53 can include a multi-step biocatalytic reaction which can be completed only in the presence of all biocatalytic species, including both the biomarkers—Amy and LDH. The reaction cascade starts with the decomposition of starch upon a cooperative biocatalytic effect of Amy and α-Glu yielding glucose (Glc) as a product. Then, Glc is converted to glucose-6-phosphate (G6P) in the presence of HK and ATP, resulting in the formation of adenosine 5′-diphosphate (ADP), which allows the next reaction step producing pyruvate (Pyr) from PEP in the presence of PK. Finally, the in situ produced Pyr allows oxidation of NADH to NAD⁺ biocatalyzed by LDH to yield decrease of the optical absorbance at λ=340 nm as the final output signal. The reactions biocatalyzed by HK and PK are needed to couple the Amy and LDH biomarker-input signals in a single biocatalytic cascade. Therefore, the NADH concentration decreases as a result of the completed reaction and should be observed only in the presence of both biomarkers, Amy and LDH. The absence of both or either input signals should result in the incomplete biocatalytic chain, thus inhibiting the NADH consumption. For the digital operation of the enzyme logic system, the presence of Amy and LDH at normal physiological concentrations in blood was defined as logic input 0, while their elevated pathological concentrations were considered as logic input 1 (see specific concentrations in the Experimental Section). Therefore, the 1,1 (Amy,LDH) input signal combination resulted in the highest activity of the biocatalytic cascade with the rapid decrease of NADH concentration. It should be noted however, that 0 logic concentrations of the input signals correspond to the normal physiological concentrations of Amy and LDH rather than their Complete absence. Thus, 0,0; 0,1 and 1,0 combinations of the input signals still allow the biocatalytic reaction to proceed, however, with a lower rate compared to the 1,1 combination of the biomarker-inputs.

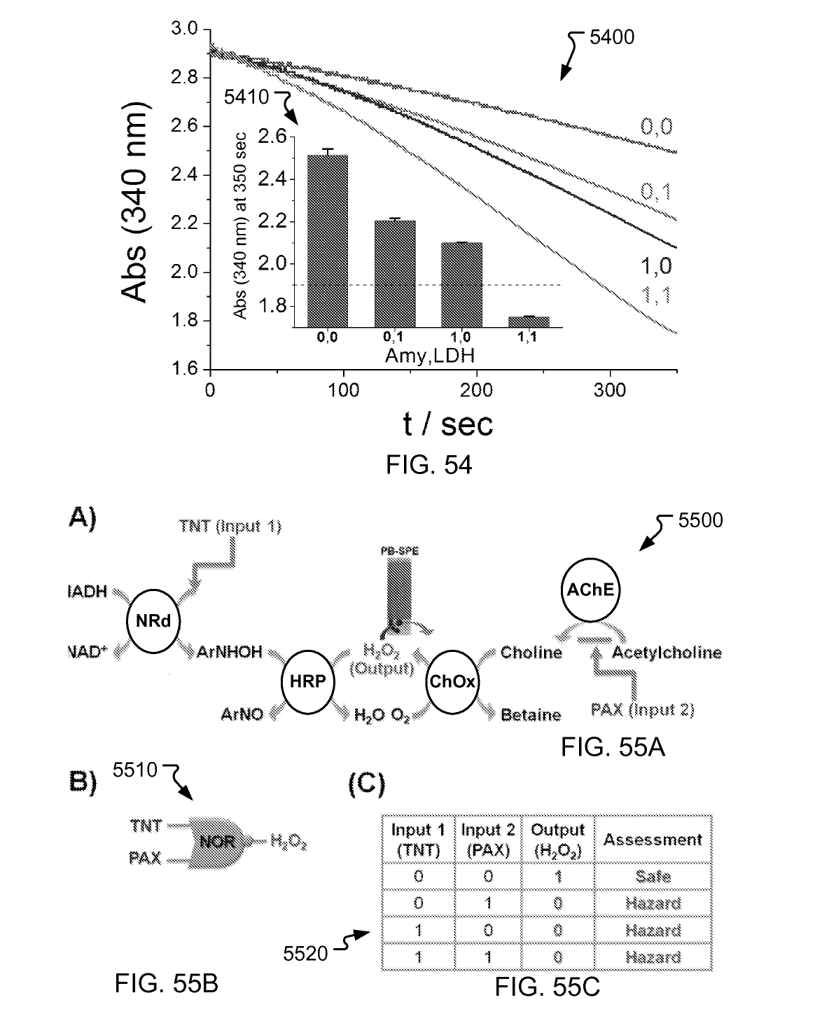

FIG. 54 shows the NADH absorbance decrease upon application of four different combinations of the input signals (5400): 0,0; 0,1; 1,0 and 1,1. FIG. 54, inset (5410), shows a bar chart for the optical output signals generated by the enzyme logic system obtained after 350 seconds of the biocatalytic reaction in the presence of various combinations of the biomarker-input signals. A threshold absorbance value of 1.9 allows clear separation of a low output signal generated upon the cooperative effect of elevated concentrations of Amy and LDH (1,1 inputs) from a high signal for the inhibited reaction when either or both input signals were applied at the logic 0 level (0,0; 0,1; 1,0 inputs). The output signal pattern corresponds to the Boolean logic operation NAND which results in the output signal 0 (low signal) only for the 1,1 combination of the inputs as shown in FIG. 53, bottom.

Numerous compounds present in the serum samples can potentially interfere with the enzymatic “machinery” of the developed analytical system. Particularly, glucose, pyruvate and ADP are intermediate products in the biocatalytic cascade and their natural presence in serum could result in a false positive signal even in the absence of the Amy input signal. The depletion of naturally existing serum glucose prior to the measurements was essential for the discrimination between the outputs generated by 0,1 and 1,1 (Amy,LDH) signals. In some implementations, the system operation was possible in the presence of pyruvate and ADP naturally existing in serum. The system operation was examined using different samples of human serum. Some minor sample-to-sample absorbance variations were observed, mostly due to the difference in the transparency of the serum samples. However, the robust operation of the bioanalytical system always allowed convenient discrimination of 0 and 1 output signals, thus providing reliable diagnostics of the pathological conditions.

Chemicals and Reagents

α-Amylase from porcine pancreas (Amy, E.C. 3.2.1.1), hexokinase from Saccharomyces cerevisiae (HK, E.C. 2.7.1.1), α-glucosidase from baker's yeast (α-Glu, E.C. 3.2.1.20), pyruvate kinase from rabbit muscle (PK, E.C. 2.7.1.40), glucose oxidase from Aspergillus niger (GOx, E.C. 1.1.3.4), lactate dehydrogenase from porcine heart (LDH, E.C. 1.1.1.27), serum from human male AB plasma, β-nicotinamide adenine dinucleotide reduced (NADH), phospho(enol)pyruvate (PEP), adenosine 5′-triphosphate (ATP, from bacterial source), glycyl-glycine (Gly-Gly), starch (soluble) and other standard organic/inorganic chemicals were purchased from Sigma-Aldrich and used as supplied without any pretreatment or purification. Ultrapure water (18.2 MΩ·cm) from NANOpure Diamond (Barnstead) source was used in all of the experiments.

Composition and Operation of the Irradiation Injury Detection System

Gly-Gly buffer, 50 mM, containing 6.7 mM Mg(CH₃CO₂)₂ was titrated with KOH to the pH value of 7.4 (note that Mg²⁺ and K⁺ cations are essential for activation of PK). The following components of the biocomputing “machinery” system were dissolved in the Gly-Gly buffer to perform NAND (Not-AND) logic operation upon activation with biomarker-input signals: NADH 0.3 mM, HK 10 U·mL⁻¹, ATP 5 mM, PK 2 U·mL⁻¹, PEP 0.5 mM. The “machinery” solution was deaerated by bubbling with Ar for 15 min prior to its use. Amy and LDH were applied as biomarker-input signals activating the biocomputing “machinery” system. Logic ‘0’ and ‘1’ levels of Amy (0.25 and 2.5 U·mL⁻¹) [28] and LDH (0.15 and 1.0 U·mL⁻¹) [29] input signals were applied in serum to realize meaningful circulating levels of these biomarkers under normal physiological and pathological conditions caused by radiation, respectively. In order to eliminate interference of glucose present in serum the following procedure was applied. Glucose was depleted from serum by pre-treatment with GOx (10 U·mL⁻¹) for 60 min. Then GOx dissolved in serum was deactivated by removing oxygen with flow of Ar for 15 min. After this pretreatment, starch (0.2 mg·mL⁻¹), α-Glu (0.5 U·mL⁻¹) (both are the parts of the system “machinery”) and the first biomarker-input—Amy (0 or 1 logic levels) were dissolved in glucose-free serum and incubated for 10 min. Then the second biomarker-input—LDH (0 or 1 logic levels) was dissolved in glucose-free serum. The serum solutions containing the biomarker-inputs in four different logic combinations (0,0; 0,1; 1,0 and 1,1) were mixed with the biocomputing “machinery” system dissolved in the oxygen-free Gly-Gly buffer (1:1 v/v ratio of serum and buffer). Optical absorbance measurements (Shimadzu UV-2450 UV-Vis spectrophotometer with a TCC-240A temperature-controlled holder) were performed continuously at λ=340 nm following the decreasing concentration of NADH upon the biocatalytic reaction activated by the biomarker-input signals. All reactions and optical measurements were performed at 37±0.2° C. mimicking physiological conditions.

Since the biomarkers, Amy and LDH, reveal relatively low specificity for the physiological changes caused by radiation only the presence of both of them at elevated concentrations can provide evidence of radiation damage. The optical changes observed in the presence of both the biomarkers appearing at pathological concentrations can be easily discriminated from the output when at least one of the selected biomarkers exists at its normal physiological concentration. The digitally operating enzyme logic system demonstrated robust performance in serum solutions in the presence of various naturally existing interferants, thus allowing the unambiguous alert-type conclusion in the YES/NO form about the presence of the radiation-caused tissue damage. Practical applications can benefit from further miniaturization and/or integration into a ‘reagentless’ solid-state optical or electrochemical device.

High-Fidelity Determination of Security Threats Via a Boolean Biocatalytic Cascade

In another aspect, techniques, apparatus and systems are described for assessing diverse security threats using a biochemical logic network system. For example, a biocatalytic cascade, emulating a NOR logic gate, is able to identify the presence of explosive compounds and nerve agents by providing a simple and rapid ‘YES’/‘NO’ alert.

For terrorist attack prevention applications, described is an easy-to-use field-deployable kit that can assess multiple chemical threats (i.e. explosives and nerve agents) in a rapid manner and alert the operator when a hazard has been encountered. A biocatalytic cascade can be implemented to assess the presence of different types of threats in a rapid and reliable manner. Such unique bioprocessing of distinct classes of threat agents is illustrated for the detection of various nitroaromatic explosives and organophosphate nerve agents using an enzyme-based logic gate. In this manner, a simple assay can replace two different time-consuming test protocols commonly used for assessing each of these unique threats. Molecular substrates have been utilized as inputs to devices that exploit chemical computation. Biocatalytic cascades that take the form of enzyme logic gates represent an attractive route for developing high-fidelity analytical devices. By forming biochemical cascades that implement logical functions, according to Boolean principles, a threshold can be established above or below which a reliable indication of an abnormal event can be detected. A distinct advantage of enzyme logic gates is embodied in their innate ability to integrate several inputs and yield a rapid assessment of complex sensing scenarios in simple ‘YES’/‘NO’ terms. Such biocatalytic logic gates can be used to yield gate operations such as XOR, NAND operations, as well as higher-order logical functions in connection with several pathophysiological situations. Described in this document is the bioprocessing of distinct classes of threat agents via a NOR gate can lead to a rapid ‘YES’/‘NO’ assessment and alert regarding the presence of either explosives and/or nerve agents in connection with the electrochemical monitoring of a single output at a disposable screen-printed electrodes (SPE).

The present system is able to detect the presence of different security-relevant analytes, compared to conventional biosensors that commonly analyze only one specific substrate. The new enzyme logic capability is evaluated and illustrated using 2,4,6-trinitrotoluene (TNT) and paraoxon (PAX) as the model inputs to a NOR gate, as well as towards the detection of the 2,4-dinitrotoluene (DNT) explosive and methyl-parathion (MPT) nerve agent. The enzyme logic approach harnesses the inherent selectivity of biocatalytic processing, thereby mitigating cross-reactivity and obviating the separation requirement, leading to a greatly simplified and rapid assay. In this fashion, a qualitative alert can be tendered to the operator in regards to the presence of harmful agents. This approach is particularly attractive to address screening scenarios aimed to determine the presence of a harmful agent rather than to identify the exact nature of such threat.

FIG. 55A shows an exemplary biocatalytic cascade 5500 used to perform NOR logic operation in connection to trinitrotoluene (TNT) and paraoxon (PAX) inputs. FIG. 55B shows an equivalent logic system 5510, and FIG. 55C shows a corresponding truth table 5520 with assessment drawn from the combinations of the input signals. As illustrated in FIG. 55A, the new logic gate employs a reaction cascade catalyzed by four enzymes: nitroreductase (NRd), horseradish peroxidase (HRP), acetylcholinesterase (AChE), and choline oxidase (ChOx). The four enzymes are leveraged as the backbone of the catalytic logic gate in order to process the TNT and PAX chemical inputs.

On the right side of FIG. 55A, H₂O₂ is produced from the catalytic coupling of AChE/ChOx and acetylcholine. On the left side, H₂O₂ is partially depleted in the presence of the nitroaromatic explosive substrate through a NRd/HRP biocatalytic cascade. An enabling feature of the cascade is the fact that the H₂O₂ level can also be reduced through the inhibition of AChE by an organophosphate nerve agent.

FIG. 55B shows the equivalent logic gate 5510 of the cascade whereby H₂O₂ is used as the output signaling compound. Accordingly, as can be seen from the truth table 5520 in FIG. 55C, a decrease in the H₂O₂ level (and hence the current output) below a selected decision threshold is indicative of a ‘Hazardous’ situation corresponding to the presence of an explosive and/or nerve agent, in line with the operation of a Boolean NOR gate. The generation of an oxidizable hydroxylamine product (from the NRd enzyme) necessitates lowering of the detection potential of the H₂O₂ output. Modification of the SPE transducer with a Prussian Blue (PB) mediator offers effective and selective peroxide detection at an extremely low potential. Based on the cyclic voltammetric characterization of the PB-modified SPE (FIG. S1A(ESI†)), a potential of −0.20 V (vs. Ag/AgCl) was selected for the detection of H₂O₂, thereby minimizing potential electroactive interference and potential false results. The resulting PB-modified electrode strip offers highly selective and sensitive measurements of H₂O₂ down to the 5 μM level (FIGS. 55B and 55C (ESI†)).

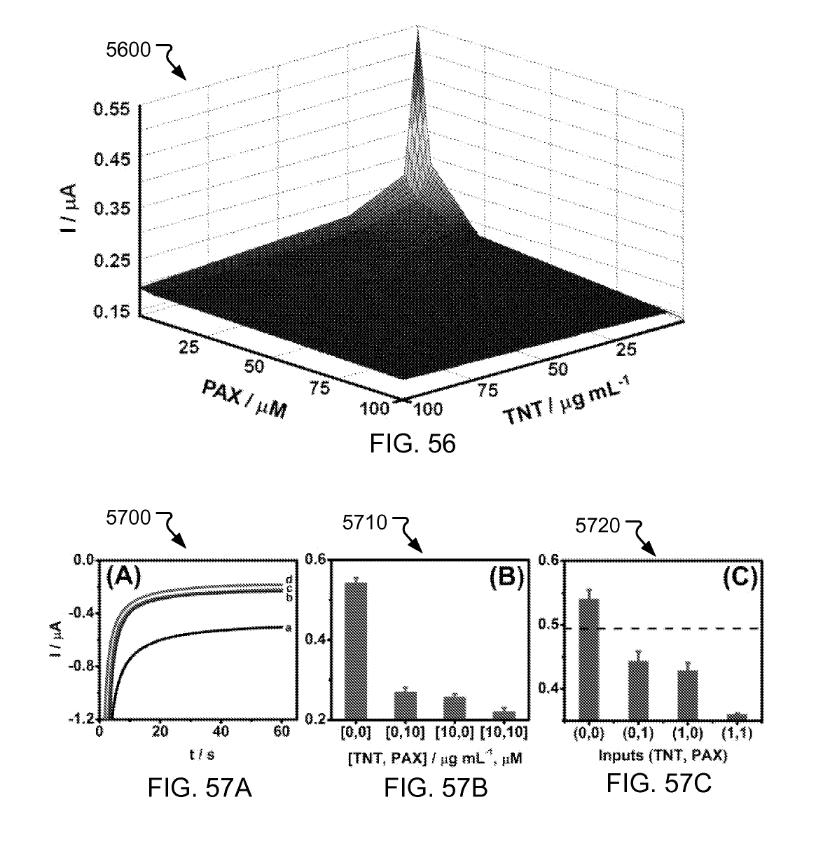

To validate the operation and optimize the performance of the constituent reactions of the logic gate, the complete system was mapped (via amperometric measurements) by ‘scanning’ the concentrations of the TNT and PAX inputs from 0 to 100 μg mL⁻¹ and 0 to 100 μM, respectively. The data points were subsequently interpolated to generate the smooth contour profile 5600 shown in FIG. 56. FIG. 56 shows exemplary Caption Gate mapping illustrating the functional dependence between the levels of the TNT and PAX inputs and the output current obtained. The output currents are sampled using chronoamperometry (at 30^{th} sec) with inputs levels of 0, 5, 25, 100 (μg mL⁻¹ and μM for TNT and PAX, respectively). The 2D linear interpolation was performed using a 20× oversampling algorithm in MATLAB. As expected for the operation of logic gates, the gradient of the current response ∇i([TNT], [PAX]) is maximized in the absence of both inputs (maximum current of 0.54 μA). In accordance with the operational functionality of a NOR gate, minor (low micromolar) increases in the levels of either of threat input yielded a dramatic decrease in the current response to less than 0.20 μA.

Four distinct operating points have been selected from the gate mapping presented in FIG. 56 for further evaluation and to validate that the behavior of the gate system at a given operating point is in agreement with its corresponding truth table. As such, chronoamperometry was performed for all four combinations of TNT/PAX corresponding to 0 and 10 μg mL⁻¹/10 μM.

FIG. 57A shows exemplary chronoamperograms of the NOR enzyme logic gate corresponding to the [TNT (μg mL⁻¹), PAX (μM)]=(a) [0,0], (b) [0,10], (c) [10,0], and (d) [01,10] inputs for three independent trials. FIG. 57B is a bar chart comparing the magnitude of the response for the four combinations of the inputs. FIG. 57C shows a bar chart of the NOR gate operating at its limit of detection for the four logic levels (TNT, PAX)=(0,0), (0,1), (1,0), and (1,1) for three independent trials. The 0 logic levels of both TNT and PAX represent the absence of these compounds in the assay while the 1 level corresponds to 1.5 μg mL⁻¹ TNT/1.25 μM PAX. The dashed line indicates the decision threshold (0.49 μA).

FIG. 57A elucidates that a NOR behavior is obtained for [TNT (μg mL⁻¹), PAX (μM)]=[0,0], [0,10], [10,0], and [10,10], with the [0,0] ‘Safe’ level isolated from the other concentrations by 0.29 μA (sampling at 30 sec). The corresponding bar chart, shown in FIG. 57B, illustrates the large dynamic range associated with the selected levels, along with a low standard deviation of 15 nA.

In order to determine a reliable limit of detection (LOD) of the system, experiments were iterated until the [0,0] level was separated by no more than six standard deviations (6σ) from any nonzero level of the inputs. This corresponded to 1.5 μg mL⁻¹ TNT and 1.25 μM PAX. Once the LOD was determined, the concentration of each of these inputs was established at logic ‘1’ while the absence of the inputs was implemented at logic ‘0’. Underscoring the system's analytical merit and utility as a detection tool, the logic levels were not established at arbitrary or convenient values but were, in fact, set at the detection limit, thereby substantiating that the system could still enable proper detection. FIG. 57C displays the chronoamperometric response of the enzyme logic NOR gate for the four combinations of the inputs (TNT, PAX)=(0,0), (0,1), (1,0) and (1,1) logic levels. As expected, the (0,0) logic level (designating ‘Safe’ conditions) is well separated from the (0,1), (1,0) and (1,1) logic levels (indicative of ‘Hazardous’ scenarios). As such, an ample 0.01 μA separation was obtained between the mean valises of the ‘Safe’ and the ‘Hazardous’ logic levels in closest proximity, hence validating the NOR functionality of the enzyme logic gate. The decision threshold (0.49 μA) is clearly indicated in the figure (taken as the halfway point between the (0,0) and (0,1) logic levels). As expected from the truth table, all combinations of the inputs that result in H₂O₂ levels exceeding this threshold are deemed to be ‘Safe’ situations whereas the generation of H₂O₂ levels below this critical value is considered to be a ‘Hazardous’ situation that warrants further action. A low standard deviation (below 16 nA) was attained for each logic level, demonstrating the robustness of the system when assessing the presence of multiple compounds.

In order to demonstrate the versatility of the enzyme logic gate and its ability to process a wide array of inputs, the study was extended for the detection of other threats, such as the DNT explosive and MPT nerve agent. FIG. 56 (ESI†) illustrates the performance of the NOR gate in connection to various combinations of the TNT, PAX, DNT, and MPT inputs. DNT yielded a decrease in the H₂O₂ concentration comparable to TNT, owing to the broad catalytic specificity of NRd toward nitroaromatic substrates. MPT was slightly less inhibitive toward AChE than PAX, resulting in a 14% increase in the H₂O₂ level (compared with equivalent levels of PAX), although the dynamic range between the levels remained high.

Various implementations have been described of an enzyme logic gate able to issue a digital alert when assessing the presence of both explosive compounds and nerve agents. Using NOR logic gate operation, an analytically-relevant threshold could be implemented in accordance with a truth table in order to provide a qualitative ‘YES’/‘NO’ alert regarding the presence of different types of threats. The concept could be readily expanded towards rapid warning of other threats in connection to different logic gates, and should be coupled with a follow-up identification of the exact threat. The high sensitivity of the PB mediator towards H₂O₂ facilitates also the direct screening of peroxide-based explosives using the same field-deployable SPE assay (but without the logic gate machinery). The ability to assess the presence of different types of hazards holds considerable promise for enhancing and simplifying a variety of security screening protocols.

Bioelectronic System for the Control and Readout of Enzyme Logic Gates

In another aspect, described is a microelectronic backbone configured specifically for the control of biocomputing sensor systems applied to diagnostic merits. The operation of the sensor systems is validated towards the rapid assessment of pathological conditions arising from soft tissue injury (STI) and abdominal trauma (ABT) using NAND and AND Boolean enzyme logic gates, respectively. The miniaturized 19×19 mm device employs a custom-designed three-electrode potentiostat coupled with an integrator, voltage amplifier, comparator, and digital logic and is easily interfaced with a screen-printed electrode contingent. By implementing an adjustable threshold comparator, a precise decision threshold could be established corresponding to pathological levels of the target biomarkers. As a result, a rapid amperometric analysis tendered the diagnosis in a straightforward ‘YES’/‘NO’ digital format via the illumination of a light emitting diode. Using low quiescent current voltage regulators, the device is able to achieve microwatt power operation and can be sustained by a single 3 V coin-cell battery for over 45 hours under continuous use. The low-power, low-cost, and miniaturized device can satisfy requirements of field-deployable logic gate amperometric sensors. Such a reconfigurable micro-/bioelectronic logic-based multi-parameter sensing system shows considerable potential for the assessment of key analytes in a multitude of relevant clinical, security, and environmental applications where go/no-go readout, rapid measurement, device miniaturization, and extended longevity on battery power are key requirements.

Techniques, apparatus and system are described for designing, developing, and evaluating a new class of electronic system specifically configured to harness the bioprocessing capabilities of biomolecular logic systems and to provide amperometric transduction of signals generated by enzyme-logic biosensors. The multivariate and versatile sensing capabilities of the concept are demonstrated, taking clinically-relevant scenarios corresponding to combat injuries as a model. This biosensor is evaluated towards the amperometric determination of pathological levels of creatine kinase/lactate dehydrogenase and lactate/lactate dehydrogenase for the diagnosis of soft tissue injury (STI) and abdominal trauma (ABT), respectively. The sensor employs enzyme cascades that imitate the operational functionality of NAND (STI) and AND (ABT) logic gates in connection with the detection of the biocatalytically-processed chemical information via disposable carbon screen printed electrodes (SPE). The biosensor system can enable a clinically-relevant switching threshold to be pre-programmed into the device and configured as needed for the intended injury/application. The results presented in this document clearly indicate the potential of the new concept for the unequivocal identification of pathophysiological conditions. A user-friendly bioelectronic sensing system, such as the one discussed here, can be well-suited to empower a non-technical operator with the ability to identify a wide array of chemical agents of importance in various clinical, security, and environmental scenarios.

Preparation of Chemicals and Reagents

Potassium phosphate monobasic (KH₂PO₄), potassium phosphate dibasic (K₂HPO₄), glycyl-glycine (Gly-Gly), magnesium acetate tetrahydrate (MgAc), potassium hydroxide (KOH), bovine serum albumin (BSA), creatine (CRTN), adenosine 5′-triphosphate disodium salt hydrate (ATP), phosphoenolpyruvic acid monopotassium salt (PEP), L(+)-lactic acid (LAC), β-nicotinamide adenine dinucleotide, reduced dipotassium salt (NADH), nicotinamide adenine dinucleotide (NAD⁺), methylene green (MG), pyruvate kinase (PK) from rabbit muscle (E.C. 2.7.1.40), creatine kinase (CK) from rabbit muscle (E.C. 2.7.3.2), and lactate dehydrogenase (LDH) from porcine heart (E.C. 1.1.1.27) were purchased from Sigma-Aldrich (St. Louis, Mo.) and were used as supplied without any pretreatment or purification. Ultra pure deionized water (18.2 M∩·cm) supplied from a Barnstead Nanopure Diamond source (Waltham, Mass.) source was used in all experiments.

A Gly-Gly buffer solution was prepared at 50 mM concentration with 6.7 mM MgAc to provide the magnesium ion activator for CK. The buffer was then titrated with 1 M KOH to create a solution with pH value of 7.95 (while providing the potassium ion cofactor essential for PK). All reagents employed in the soft tissue injury (STI) gate were prepared in this buffer solution. MG was employed as a mediator to enable the low-potential oxidation of NADH.

A potassium phosphate buffer solution was prepared at 50 mM concentration by mixing precise mole fractions of KH₂PO₄ and K₂HPO₄ in order to achieve a pH value of 7.10. All reagents employed in the abdominal trauma (ABT) gate were prepared in this buffer solution. MG was employed as a mediator to enable the low-potential oxidation of NADH.

Electronic Components

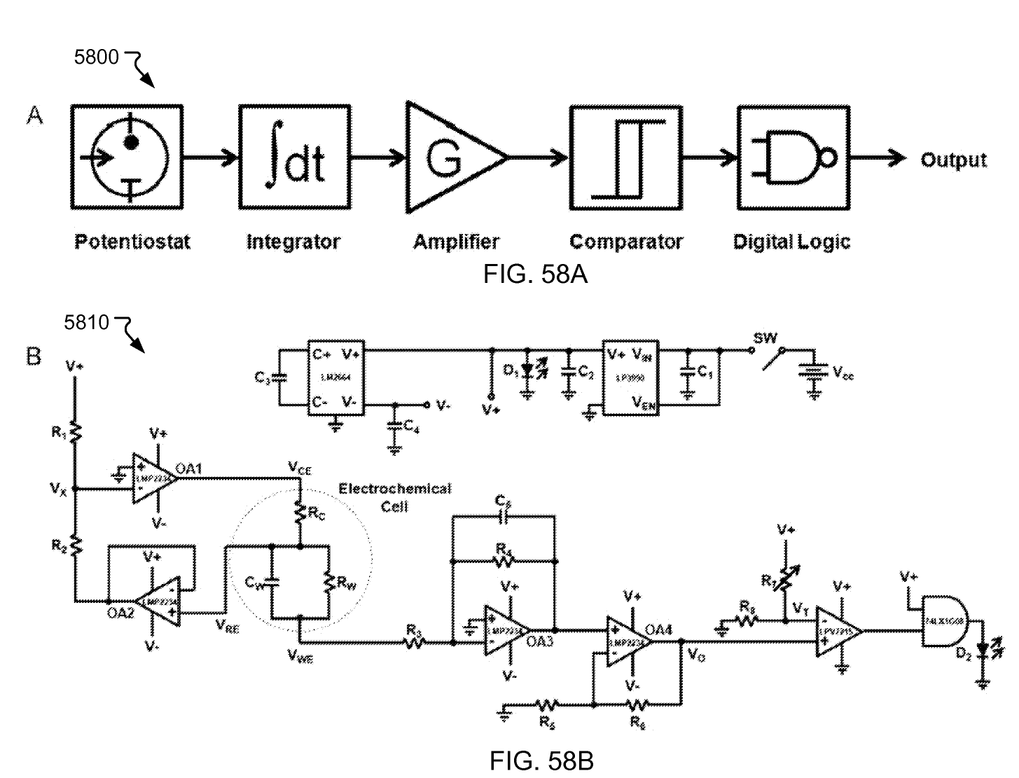

A linear voltage regulator (LP3990), switched capacitor voltage converter (LM2664), quad micropower precision amplifier with CMOS input (LMP2234), and micropower comparator with CMOS input (LPV7215) were procured from National Semiconductor (Santa Clara, Calif.). Single CMOS two-input AND (74LX1G08) and NAND (74LX1G00) gates were obtained from STMicroelectronics (Geneva, Switzerland). A CR1025 3V manganese dioxide lithium-ion coin cell battery was purchased from Panasonic Corp. (Osaka, Japan). All other passives (resistors, potentiometers, capacitors, LEDs, switch, and battery holder) were acquired from Digikey (Thief River Falls, Minn.). Block-level (5800) and circuit-level diagrams (5810) are illustrated in FIGS. 58A and 58B respectively. Specifically, FIG. 58A is a process flow diagram outlining an equivalent functional behavior (5800) of an exemplary microelectronic sensing system. FIG. 58B is an exemplary circuit-level schematic (5810) of the supporting electronics designed for the analysis of abdominal trauma. In order to realize correct logic operation, the CMOS AND logic gate in the figure is replaced with a CMOS NAND logic gate for the readout of the soft tissue injury system.

The linear voltage regulator was configured to generate a +1.8 V supply rail from the 3 V battery, which was fed into the switching voltage converter, thereby yielding a −1.8 V rail to implement fully differential voltage compliance at the potentiostatic unit. A NAND gate was employed for the STI experiments in order to invert the logic output generated by the comparator and consequently drive the status indicator LED. For the ABT experiments, an AND gate was used in the place of the NAND gate to drive the status indicator LED. The selection of resistors employed in the potentiostat was as follows: R₁=R₄=R₆=R₈=1 MΩ, R₂=1 kΩ, R₃=43 kΩ, R₅=100 kΩ, and R₇ was adjusted in accordance with the switching threshold required by the application. The selection of capacitors employed in the potentiostat was as follows: C₁=C₂=1 μF (used for regulator stability), C₃=C₄=3.3 μF (switched converter charge storage), and C₅=1 μF (low-pass filtering).

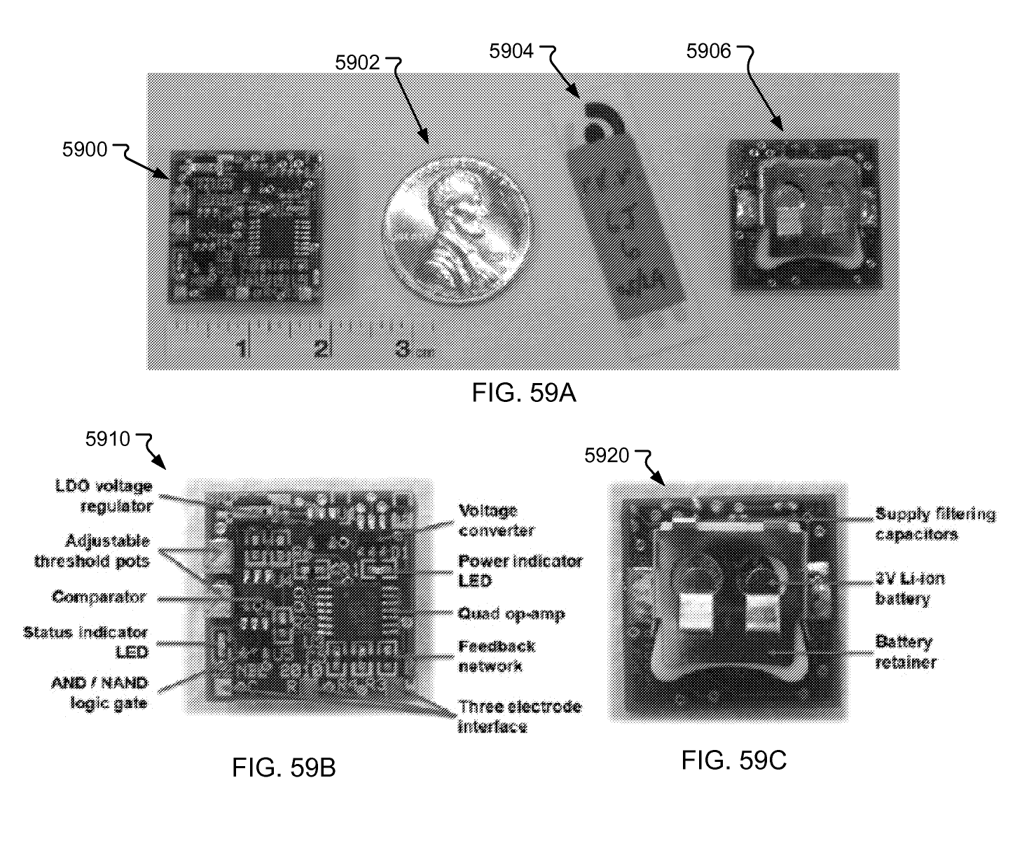

A 19×19 mm 4-layer printed circuit board (PCB) was custom-designed using an AutoCAD® electrical layout editor and outsourced for fabrication. The PCB consisted of separate power and ground planes as well as a battery holder on the reverse side. A digital logging multimeter was employed for the electrical measurements. FIG. 59A is an image of an exemplary microelectronic system 5900, 5906 (US 1¢ 5902 and screen printed three-electrode strip shown 5904 for size comparison). FIG. 59B is an image of an obverse detail of an exemplary microelectronic system indicating the locations of the constituent components on the PCB. FIG. 59C is an image of a reverse detail of an exemplary microelectronic system indicating the locations of the constituent components on the PCB.

Electrode Design and Fabrication

The fabrication of the ceramic-based screen-printed electrode-sensor is detailed: A laser-scribed alumina substrate was obtained from CoorsTek Inc. (Golden, Colo.). An Ag/AgCl-based ink from Ercon Inc. (E2414) was employed to define the conductive underlayer as well as the reference electrode and printed directly onto the substrate. A carbon-based ink (Ercon E3449) was then overlaid on the conductor to define the working and counter electrode geometry. Finally, an insulator ink (Ercon E6165) was overlaid on the Ag/AgCl and carbon layers to insulate all but the contact pads and the upper segment of the electrodes. In each of the three aforementioned processing steps, a Speedline Technologies MPM-SPM screen printer was used to print the pattern onto the ceramic substrate using a custom-designed stainless steel stencil. Subsequent to the printing process, the patterned substrate was cured in a temperature-controlled convection oven (SalvisLab Thermocenter) at 120° C. for 20 min and cleaved into test strips for single use. Each screen printed three-electrode strip consisted of a circular carbon working electrode (geometrical area: 3 mm²) inscribed in a hemispherical counter (area: 10 mm²) and reference electrode (area: 2 mm²).

Selection of the Biomarkers and Clinical Relevance

Among the plethora of relevant biomarkers implicated in soft tissue injury (STI), serum levels of CK and LDH become noticeably elevated under circumstances where muscular exertion, fatigue, injury, and trauma are sustained. CK, a specific indicator of rhabdomyolysis, has been shown by Kaste et al. to increase from an average serum level of 100 U/L under normal physiological conditions to around 710 U/L when an STI event has been incurred. Likewise, circulating levels of LDH, an enzyme frequently employed for the determination of tissue breakdown and hemolysis, can increase markedly from around 150 U/L under normal circumstances to over 1000 U/L under pathological states.

Abdominal trauma (ABT), whether of the penetrating or blunt variety, represents a common class of combat injury whereby one or multiple organs in the abdominal cavity are ruptured or otherwise damaged. In such scenarios, serum LAC and LDH are among the biomarkers of choice in the clinical setting when assessing organ damage and malfunction. Whereas LDH exhibits a similar concentration profile as indicated above under this class of injury, LAC has been shown to increase by Hara et al. from 1.6 mM to 6.0 mM.

Composition of the Logic Gates and Protocol

In both the systems under investigation (STI and ABT), the normal physiological concentrations of the selected biomarkers were employed as digital ‘0’ input signals, while the elevated pathological concentrations were defined as ‘1’ input signals. Thus, the systems were evaluated at four different combinations of the input signals: (0,0), (0,1), (1,0), and (1,1), where only the last combination corresponded to pathological scenarios, while the three other logic combinations reflected normal conditions or other irrelevant physiological anomalies. In addition to the binary levels of the input injury biomarkers, other reagents were experimentally optimized and employed at constant concentrations. These supporting chemicals served as the system “machinery” and therefore performed the biochemical analysis of the logic input signals.

The STI experiments were conducted by employing 0.3 mM NADH, 0.5 mM PEP, 2 mM ATP, 15 mM CRTN, 0.3 mM MG, 2000 U/L PK, 100 U/L (‘0’)/710 U/L (‘1’) CK, and 150 U/L (‘0’)/1000 U/L (‘1’) LDH in a 50 μL sample volume. All reagents were mixed in a tube and subjected to a 180-sec incubation at 37° C. in a heatblock. Following this incubation period, the solution was dispensed on the electrode surface and a chronoamperogram was subsequently initiated whereby a working electrode potential of 0.0 V (vs. Ag/AgCl) was maintained for 60 sec.

The ABT experiments were conducted by employing 10 mM NAD⁺, 1 mM MG, 1.6 mM (‘0’)/6.0 mM (‘1’) LAC, and 150 U/L (‘0’)/1000 U/L (‘1’) LDH in a 50 μL sample volume. All reagents were mixed in a tube and subjected to a 180-sec incubation at 37° C. in a heatblock. Following this incubation period, the solution was dispensed on the electrode surface and a chronoamperogram was subsequently initiated whereby a working electrode potential of 0.0 V (vs. Ag/AgCl) was maintained for 60 sec.

References to digital logic gates in bold typeface (ie. NAND) represent enzyme-based manifestations of logic gates. On the other hand, references without a bold typeface (ie. NAND) represent their CMOS counterparts.

Design of the Electronic Backbone

The new electronic architecture has been designed to control biocomputing systems applied to diagnostic merits. To simplify analysis, a Randles-Ershler equivalent R-C circuit model is employed to emulate the electrical behavior of the electrochemical system, as displayed in FIG. 58B. This model consists of a parallel capacitor (C_{w}, corresponding to the double layer capacitance arising from the accumulation of a net surface charge at the working electrode) and resistor (R_{w}, corresponding to the charge transfer/Faradaic resistance arbitrated by the electroactive species) in series with another resistor (R_{c}, the total solution resistance). The potentials at the working, counter, and reference electrodes are denoted as V_{WE}, V_{CE}, and V_{RE}, respectively.

As illustrated in FIG. 58B, the potentiostatic unit consists of two LMP2234 precision instrumentation operational amplifiers (OA) configured in the following arrangement: control amplifier OA1 amplifies the differential voltage seen between node Vₓ and ground (with gain A) and supplies current through the counter electrode. Upon sensing a voltage generated at the reference electrode, OA2, a voltage follower/buffer, syncs sufficient current through R₂ in order to maintain its output voltage at the input (V_{RE}) value. In turn, Vₓ is adjusted and the output potential/current of OA1 is modified accordingly. The control amplifier thus functions as a voltage-controlled current source that delivers sufficient current to maintain the reference electrode at constant potential and arbitrate the electrochemical reaction. In implementing negative feedback, it is imperative that OA1 be able to swing to extreme potentials to allow full voltage compliance required for chemical synthesis. Furthermore, it is crucial that OA2 possesses very high input impedance in order to draw negligible current; otherwise the reference electrode may deviate from its intended operating potential. In practice, the use of precision instrumentation amplifiers possessing 20 fA of input bias current enables unabated operation to the sub-picoampere level, which is suitable for nearly all electrochemical studies.

Employing the equivalent circuit model of the electrochemical cell, the current through the cell may be expressed in the frequency domain as

$${i}_{cell} ( ω ) = \frac{{AV}_{X} - {V}_{WE}}{{R}_{C} + \frac{{R}_{W}}{1 + jω {R}_{W} {C}_{W}}} . \qquad ( 1 )$$

and the voltage established at the reference electrode is given by the relation

$${V}_{RE} ( ω ) = {V}_{WE} + {i}_{CELL} ( \frac{{R}_{W}}{1 + jω {R}_{W} {C}_{W}} ) . \qquad ( 2 )$$

where i_{cell} represents the current flowing from the counter electrode to the working electrode. The voltage at the counter electrode will follow the potential seen at node Vₓ,

$$\begin{matrix}{V}_{CE} ( ω ) = {AV}_{X} = A \frac{( ζ - 1 ) {V}_{WE} - \frac{{R}_{2}}{{R}_{1}} {V}^{*}}{( ζ A - 1 - \frac{{R}_{2}}{{R}_{1}} )} . \\ and & ( 3 ) \\ ζ = \frac{1}{1 + \frac{{R}_{C}}{{R}_{W}} ( 1 + jω {R}_{W} {C}_{W} )} . & ( 4 )\end{matrix}$$

The potential at the working electrode (with respect to the reference) must be specified as it is a crucial parameter in electrochemistry that dictates the activation of the electroactive species. More specifically, the application of a suitable potential at the working electrode will ensure that the electroactive substance within the medium is oxidized or reduced. Consequently, this will yield a Faradaic current proportional to the concentration of the analyte by the Cottrell equation. Synthesizing the above expressions, network analysis may be performed, yielding the frequency-domain voltage at the working electrode,

$${V}_{WE} ( ω ) = \frac{- {R}_{2} {R}_{3}}{{R}_{1} ( {R}_{3} + \frac{{R}_{W}}{1 + jω {R}_{W} {C}_{W}} )} {V}^{+} \qquad ( 5 )$$

and the DC response can be evaluated

$${V}_{WE} ( DC ) = \frac{- {R}_{2} {R}_{3}}{{R}_{1} ( {R}_{3} + {R}_{W} )} {V}^{+} . \qquad ( 6 )$$

The above relations indicate that, for a system with R_{W}>>R₃, the potential at the working electrode can be adjusted by modifying the ratio between R₂ and R₁. Equations 5 and 6 elucidate that the working electrode voltage is inversely proportional to the Faradaic resistance and therefore directly proportional to the Faradaic current arising from the electrochemical reaction. Accordingly, by the Cottrell equation, the concentration of the analyte can be extrapolated and should be linearly related to the signal arising at the working electrode. It is important to note that R₃ is selected to enable best noise performance at the expense of response time. Increasing this value will enable lower noise readings, but longer response times. For quasi-real-time measurements where a reading is recorded on a non-continuous basis at some fixed interval, it is appropriate to employ a moderate R₃ resistance in order to enable the highly sensitive detection of the analyte.

OA3, an integrator (another LMP2234 precision instrumentation operational amplifier), implements a low-pass filtering operation and provides low-noise gain to the signal arising at V_{WE}. R₄ provides the necessary feedback at DC/low frequencies (where C₅ has large reactance) to maintain a stable output at the correct value. With suitable choice of R₄ and C₅, the integrator can mitigate the high-frequency oscillation/instability induced by the capacitive loading of the potentiostat. The output of the integrator is subsequently amplified by OA4 (LMP2234), a non-inverting voltage amplifier, in order to provide additional gain to bring the signal to rail levels. The output voltage of the final amplifier stage is given by

$$\begin{matrix}{V}_{o} ( ω ) = \frac{{R}_{2} ( \frac{{R}_{4}}{1 + jω {R}_{4} {C}_{5}} )}{{R}_{1} ( {R}_{2} + \frac{{R}_{W}}{1 + jω {R}_{W} {C}_{W}} )} ( 1 + \frac{{R}_{6}}{{R}_{5}} ) {V}^{+} , \\ {ω}_{c} = \frac{1}{{R}_{4} {C}_{5}} \\ and & ( 7 ) \\ {V}_{o} ( DC ) = \frac{{R}_{2} {R}_{4}}{{R}_{1} ( {R}_{a} + {R}_{W} )} ( 1 + \frac{{R}_{6}}{{R}_{5}} ) {V}^{+} . & ( 8 )\end{matrix}$$

As can be deduced from Equations 7 and 8, the output voltage of OA4 is inversely proportional to the Faradaic resistance and therefore directly proportional to the Faradaic current arising from the electrochemical reaction. The output voltage V₀ thus serves as an indicator of the amount of electroactive analyte present in the system.

Following the analog signal processing, V₀ is incident on a comparator, which compares this value with a pre-established voltage V_{T} that is implemented by adjusting the potentiometer R₇ in relation to a fixed resistor R₈. In the event that V₀ exceeds V_{T}, the comparator will output the full rail voltage (logical ‘1’); otherwise, the output of the comparator will be at ground potential (logical ‘0’). In this manner, the device operates as a 1-bit analog-to-digital converter with an adjustable switching threshold.

The output of the comparator is channeled to one of the inputs of a CMOS logic gate and the other input is tied to the supply voltage. The CMOS logic gate serves to source sufficient current to drive an LED. An AND logic gate is employed when the enzyme logic gate implements the AND operation. Accordingly, when the output of the potentiostat and supporting analog subsystem exceeds the pre-programmed threshold level, the comparator outputs a ‘high’ (logical ‘1’) voltage, hence driving the output AND gate high and thereby illuminating the LED. Likewise, a NAND logic gate is utilized when the enzyme logic gate implements the NAND operation. In this case, the presence of a sufficient level of analyte would cause the output of the potentiostat and supporting analog subsystem to fall below the pre-programmed threshold level. As a consequence, the output of the comparator would fall to the ground potential (logical ‘0’), hence driving the output of the NAND gate high and resulting in the illumination of the LED.

With the above implementation of the electronic hardware, the complete sensor system consumed 218 μA of current at 3.0V, and thus the total power dissipation was 654 μW. Given a typical 30 mAh 3V CR1025 coin cell battery, such a system could be sustained for over 45 hours under continuous use.

High-Fidelity Readout of Soft Tissue Injury

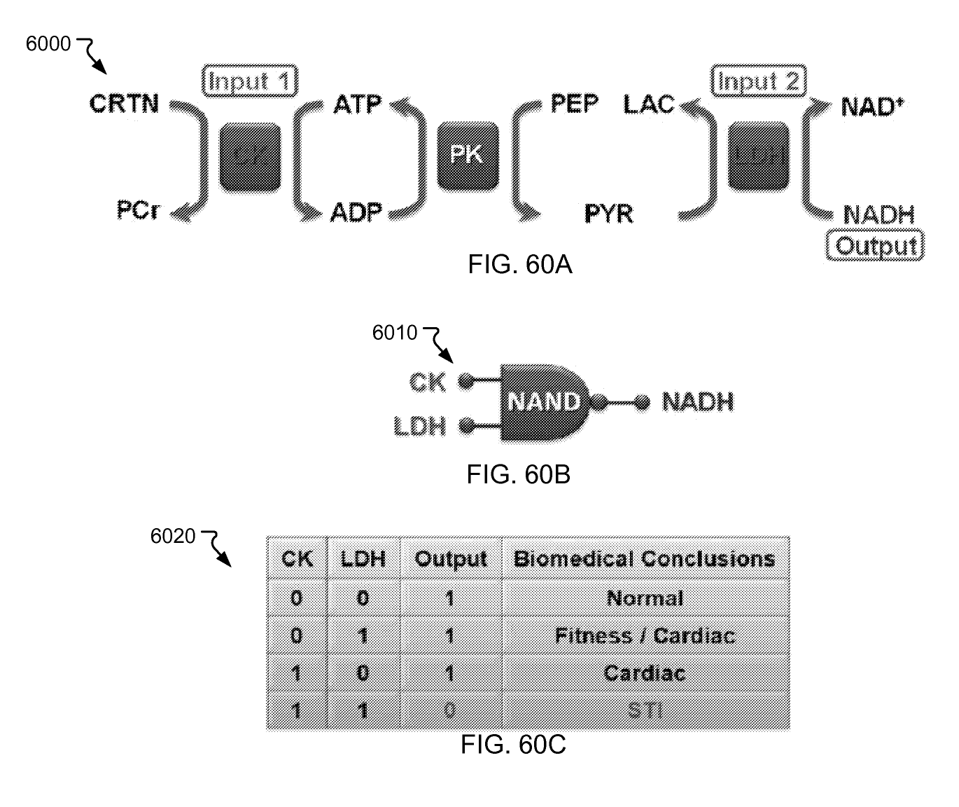

With a robust electronic backbone in place, the micro-/bioelectronic sensor system was applied towards the detection of STI with an enzyme-based NAND gate. FIG. 60A shows an exemplary biocatalytic cascade 6000 instigated by creatine kinase (CK) and lactate dehydrogenase (LDH) emulating NAND operation. FIG. 60B is a block diagram 6010 showing an exemplary equivalent logic system. FIG. 60C is a corresponding truth table 6020 with biomedical conclusions, drawn from the combinations of the input signals.

FIG. 60A illustrates the biocatalytic cascade 6000 whereby the enzyme inputs CK and LDH are processed to yield NADH as an output. The equivalent logic gate 6010 is shown in FIG. 60B. Upon the detection of abnormally high levels of both CK and LDH, the quantity of NADH present in the chemical system would decrease, as is evident from the truth table 6020 shown in FIG. 60C, thereby triggering the illumination of the LED.

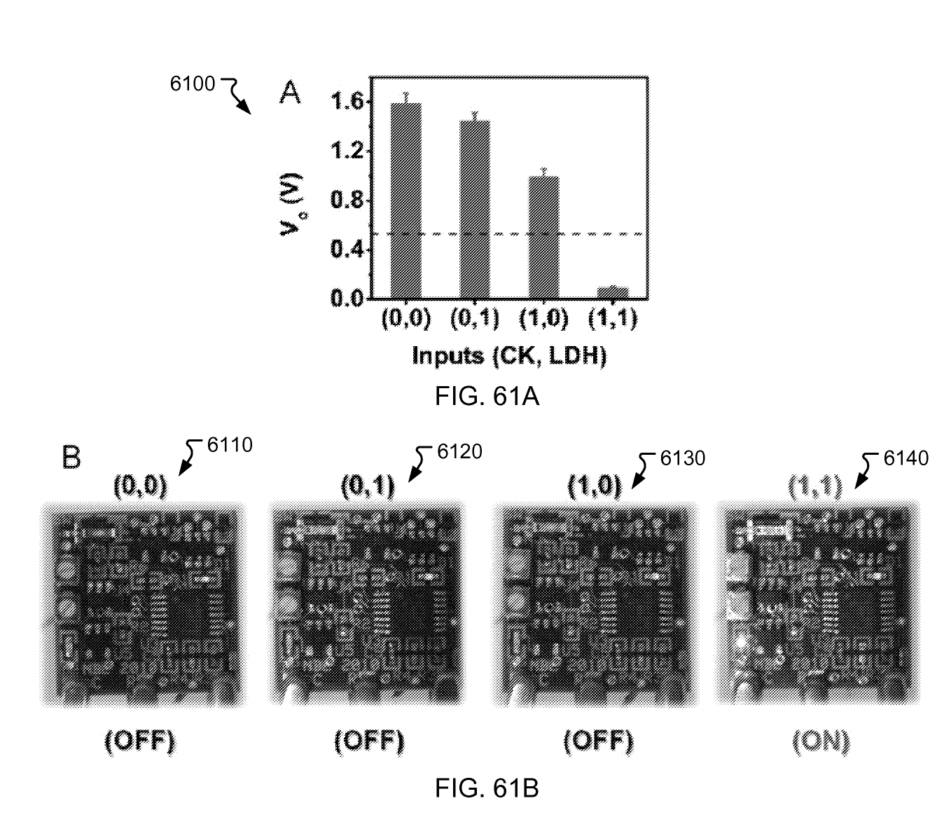

In order to resolve the proper switching threshold that would indicate the occurrence of an STI event, the sensor was evaluated towards the operation of the NAND gate under four input logic combinations. FIG. 61A is a bar chart 6100 featuring the NAND logic operation for the corresponding combinations of input signals. Electrochemical measurements were performed at E=0.0 V vs. Ag/AgCl. Dashed lines indicate the decision threshold for the realization of NAND gate operation. FIG. 61B is images 6110, 6120, 6130 and 6140 of an exemplary microelectronic system under the application of various combinations of the input biomarkers CK and LDH. Only the pathological scenario involving high levels of both CK and LDH corresponding to the (1,1) logic level rendered an output logic 0, resulting in the illumination of an LED.

FIG. 61A displays a bar chart 6100 obtained at the SPE by the NAND gate upon the application of all four of the input logic combinations for three independent trials. At 60 sec sampling time, the difference in mean voltage between the pathological logic level (1,1) and the physiological logic level in closest proximity (1,0) was 0.898 V. An exceptionally low standard deviation of less than 90 mV was maintained at every logic level.

Given the need to institute a threshold for the presentation of an affirmative diagnosis, the decision threshold was established as the midway point between the (1,1) and (1,0) logic levels, 0.535 V. As such, potentiometer R₇ was adjusted to 297 kΩ and accordingly a voltage divider (with respect to R₈) was implemented to realize a reference voltage (0.535 V) for the comparator. In pathophysiological circumstances that resulted in an output voltage V₀ below this threshold voltage V_{T}, light emission from the LED ensued. FIG. 61B displays photographs 6110, 6120, 6130 and 6140 of the sensor under the application of the (0,0), (0,1), (1,0), and (1,1) logic levels once the programmable threshold was established. Clearly, only the pathological case (1,1) resulted in the illumination of the LED, thereby alerting the operator that an STI event has occurred and demonstrating the system's unambiguous assessment of pathophysiological state.

High-Fidelity Readout of Abdominal Trauma

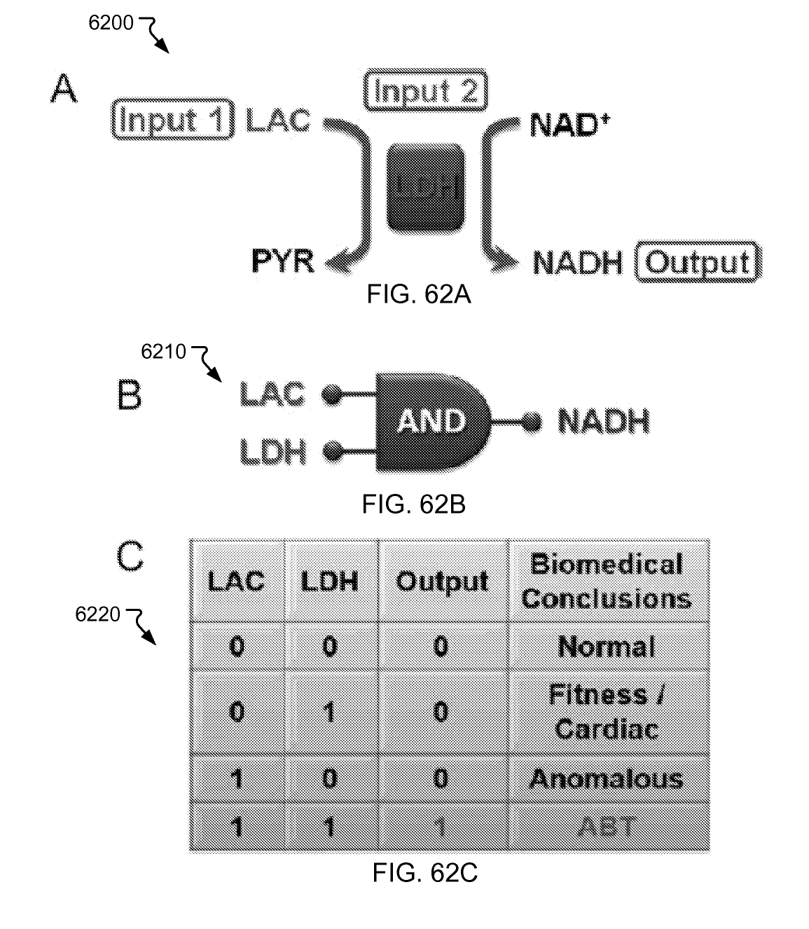

Following the system-level validation of the micro-/bioelectronic sensor towards the evaluation of STI, the sensor was subsequently applied towards the detection of ABT with an enzyme-based AND gate. The biocatalytic cascade 6200 is displayed in FIG. 62A whereby the input biomarkers LAC and LDH are processed to yield NADH as an output. The equivalent logic gate 6210 is shown in FIG. 62B. In contrast to the STI system, upon the detection of abnormally high levels of both LAC and LDH, the quantity of NADH present in the chemical system would increase. This trend can be inferred from the truth table shown in FIG. 62C, and this process can be monitored by the operator via an LED display.

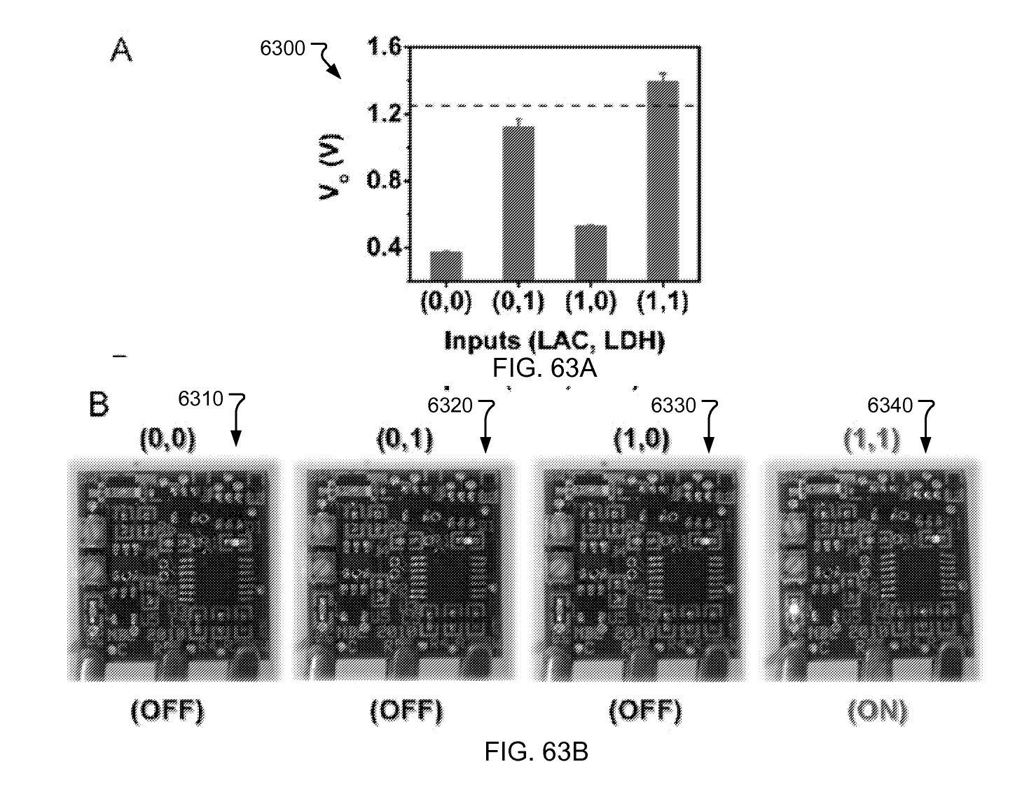

As in the STI system, in order to resolve the proper switching threshold that would indicate the occurrence of an ABT event, the sensor was evaluated towards the operation of the AND gate under four input logic combinations. FIG. 63A displays a bar chart 6300 obtained at the SPE by the AND gate upon the application of all four of the input logic combinations for three independent trials. At 60 sec sampling time, the difference in mean voltage between the pathological logic level (1,1) and the physiological logic level in closest proximity (0,1) was 0.267 V. An exceptionally low standard deviation of less than 60 mV was maintained at every logic level.

In order to achieve the highest-fidelity diagnosis possible, the decision threshold was established at the halfway point between the (1,1) and (0,1) logic levels, 1.254 V. As such, potentiometer R₇ was adjusted to 697 kΩ and accordingly a voltage divider (with respect to R₈) was implemented to realize a reference voltage (1.254 V) for the comparator. In pathophysiological scenarios that resulted in an output voltage V₀ exceeding this threshold voltage V_{T}, light emission from the LED ensued. FIG. 63B displays photographs 6310, 6320, 6330 and 6340 of the sensor under the application of the (0,0), (0,1), (1,0), and (1,1) logic levels once the programmable threshold was established. Clearly, only the pathological case (1,1) resulted in the illumination of the LED, thereby alerting the operator that an ABT event has occurred and again demonstrating the system's diagnostic integrity and utility as a versatile backbone for the readout of enzyme logic gates.

The two systems presented here are expected to perform as intended for a large majority of the population in circumstances where the biomarker levels fall within clinically-established ranges. However, in both scenarios, owing to the variable extent of afflictions and the presence of a myriad of sources of potential interference, the execution of a large clinical study that integrates various degrees of injury is imperative in order to select the most optimal threshold level for the general population.

A microelectronic backbone has been designed towards the control and readout of digital biosensors with built-in enzyme logic and evaluated towards the diagnostic assessment of soft tissue injury and abdominal trauma. Upon sensing pathological levels of the biomarker pairs CK/LDH and LAC/LDH using NAND and AND enzyme logic gates, respectively, the sensor rendered an affirmative diagnosis via the illumination of an LED. Leveraging the intrinsic biochemical processing capabilities of enzyme logic gates, the sensor is designed to harness the quantized and binary nature of the chemical outputs generated by these gates, hence enabling decisive operational merits such as unambiguous ‘YES’/‘NO’ readout, rapid measurement, small size, and extended battery lifetime. With a detailed understanding of the analytical capabilities of enzyme logic gates and suitable methods of electronic transduction, high-fidelity biocomputing sensing systems can be constructed and implemented in a straightforward manner. In this vein, detailed multivariate chemical analysis can be tendered regardless of the nature or complexity of the enzyme logic gates utilized. The micro-/bioelectronic sensing concept is the first example, to the best of our knowledge, of the development of an electronic system specifically tailored for the evaluation of biocomputing systems applied to diagnostic merits. The low-power, low-cost, and miniaturized embodiments of the sensor system make the design particularly attractive for diverse field operations. The sensor system can empower the non-technical end-user with the ability to assess the presence of chemical species in various clinical, security, and environmental scenarios in a straightforward and convenient manner.

Immuno-Logic Strip for Diabetes Detection

The urgent diagnoses and treatment of diabetes necessitates the rapid development of various devices ranging from disposable single use strips to rather complicated devices for continuous monitoring.

Glucose, hemoglobin A1c (HbA1c), and fructosamine have been routinely employed in the assessment of disease progress as clinically established indicators. While blood glucose levels can significantly vary within minutes, HbA1c is elevated in proportion to the average glucose level during the erythrocyte lifespan, reflecting the blood glucose concentration of the preceding 2-3 months. Clinical methods for the determination of HbA1c include several separation steps based on charge and structural characteristics, followed by colorimetric/electrochemical quantification of glycated hemoglobin.

According to the International Expert Committee Report on the Role of the A1C Assay in the Diagnosis of Diabetes “There is no single assay related to hyperglycemia that can be considered the gold standard, as it relates to the risk for microvascular or macrovascular complications.” In our approach we propose simultaneous biochemically coupled detection of two key diabetes markers instead of a standalone detection of glucose or HbA1c. The redundancy of the measurement will be maximized, when glucose and HbA1c are present simultaneously (Boolean AND logic) in elevated concentrations.

Increased occurrence of HbA1c in blood, as specific evidence of diabetes, is conditioned by elevated glucose levels. In this way, the coupling of glucose detection (marker 1) with the presence of HbA1c (marker 2) relays an important diagnostic information resulting from the simultaneous presence of both markers. Individual and even parallel analysis, within one device will miss redundancy of our approach of incorporating both analytes into the same reaction pathway. Thus, the approach is aimed at addressing the physiological background of diabetes and its reliable recognition with maximal redundancy.

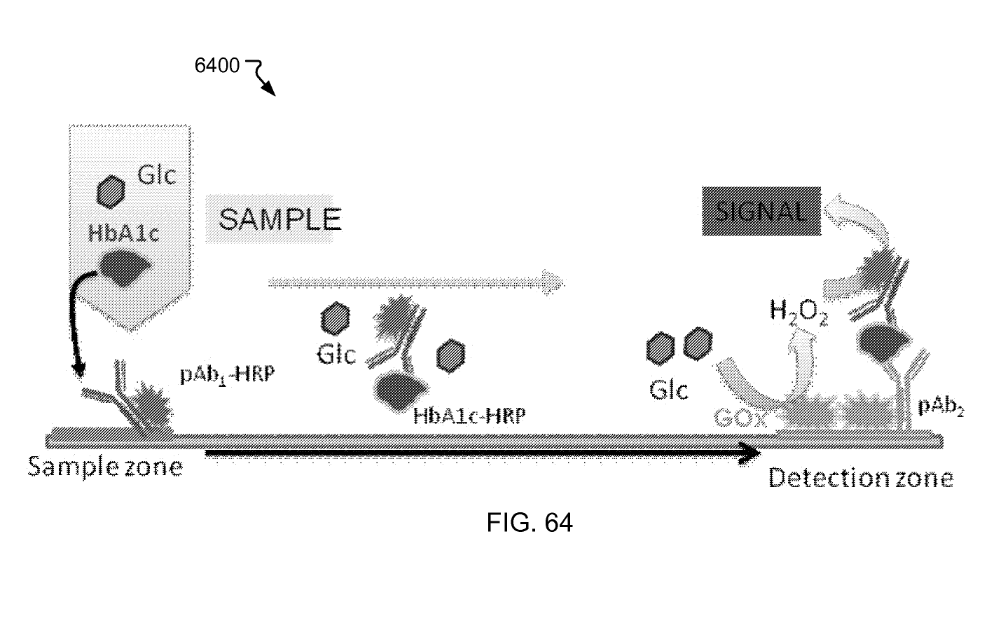

FIG. 64 shows an exemplary schematic performance 6400 of the proposed two-marker strip based on the immuno-recognition followed by biocatalytic process. The presented detection system is based on the accurate and established immuno-recognition of HbA1c molecules coupled with glucose detection via a biocatalytic cascade as shown in FIG. 64. Contrary to the commercially established point-of-care sensor systems based on a single marker approach, the proposed system will have an advantage of improved decisive ability and diagnostic fidelity due to the enhanced detection cascade.

The described immuno-logic test strip is a single use unit allowing the assessment of an individual blood sample as shown in FIG. 64. The strip contains an anti-HbA1c (reporter) polyclonal antibody labeled with a horseradish peroxidase (HRP), which is deposited (dried) in a sampling zone patch. Another anti-HbA1c analyte-specific (capture) polyclonal antibody is immobilized on the surface of the detection zone together with glucose oxidase (GOx) and dried redox dye. When a liquid sample (blood droplet) is placed onto the sample zone, the solution solubilizes the labeled reporter antibody, which binds to the HbA1c (analyte 1). This analyte-antibody complex flows with the liquid sample laterally along the surface of the strip. When the complex passes over the detection zone where the capture antibody has been immobilized, the complex binds to the capture antibody and is trapped, thus accumulating at the capture zone on the strip. In this way, HbA1c labeled with HRP is concentrated in the detection zone. Simultaneously, immobilized GOx converts glucose (analyte 2) present in the sample to hydrogen peroxidse, which serves as the substrate for the HRP-labeled reporter antibody present on the HbA1c molecule. This process results in the oxidation of the dissolved redox dye. The decreased (physiological) concentration of either analyte (negative sample) reduces the detection cascade efficiency, resulting in decrease of oxidized dye concentration, i.e. output signal. In this case, the enzymatic cascade output can be gradually reduced by the careful optimization of reaction conditions, as recently presented by our research groups [6-9]. Due to the fact that the sample (blood droplet) is colored, a control zone (sample flow indication) does not need to be incorporated in the strip.

Conventional strips (for single analyte) can usually provide a YES/NO determination of the presence of the target analyte or a threshold (semi-quantitative) result, typically within minutes. When compared with traditional biosensor concepts, this methodology merges the inherent redundancy and robustness of a Boolean logic approach in reaching ‘True’ or ‘False’ decisions with the specificity and dynamic range associated with biocatalytic processing.

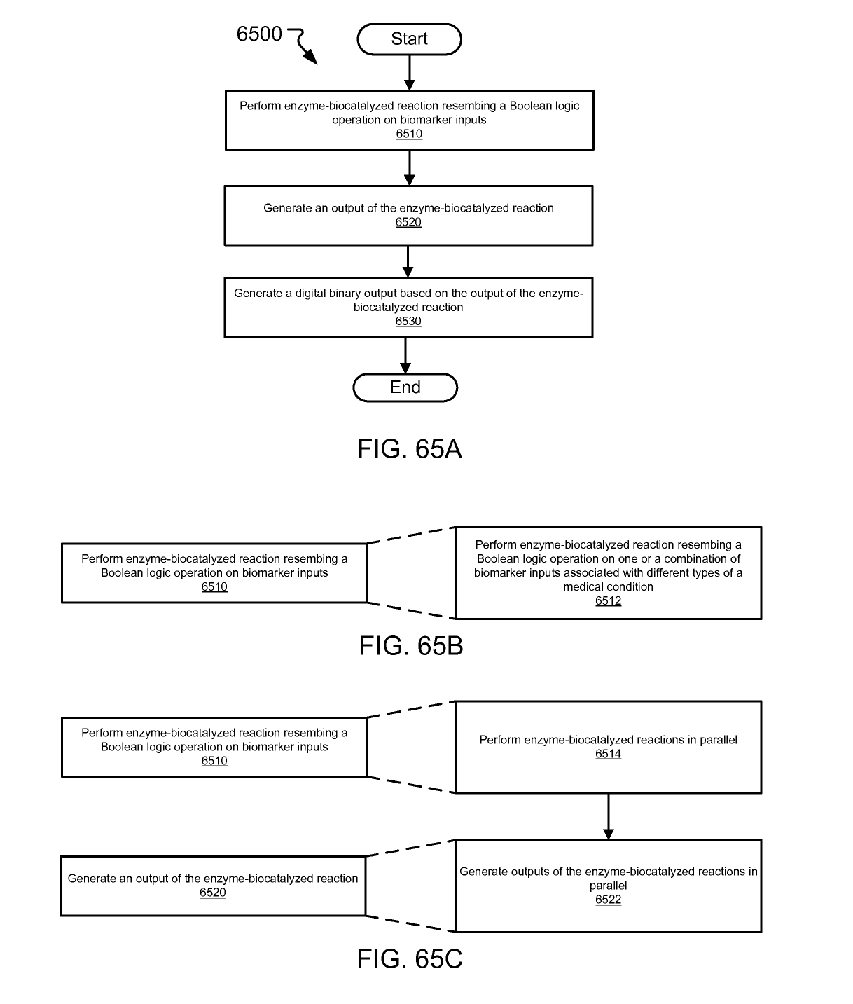

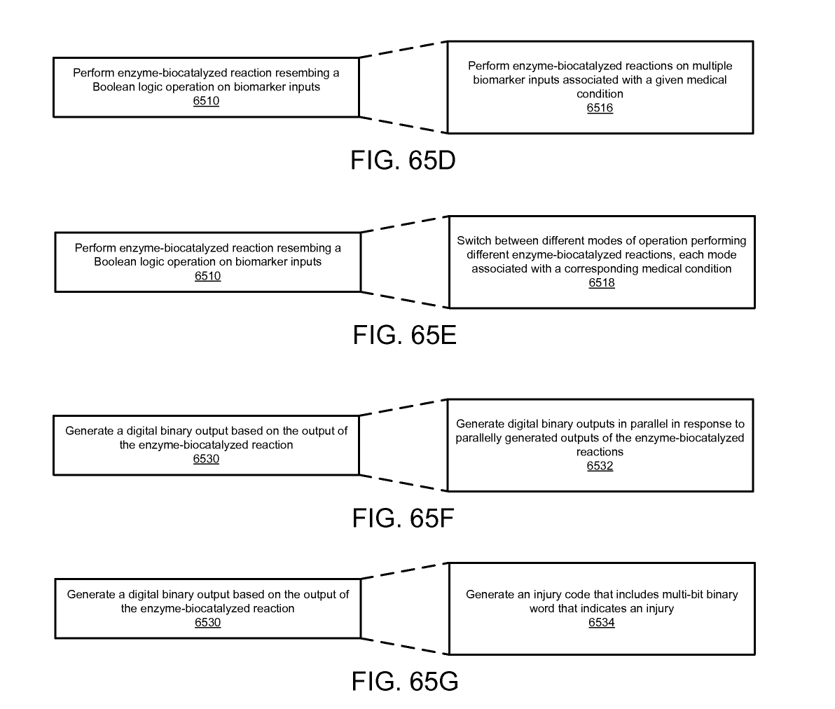

FIGS. 65A, 65B, 65C, 65D, 65E, 65F and 65G are process flow diagrams of an exemplary process for implementing enzyme-logic based diagnosis. The process 6500 can include operating a network of enzyme-biocatalyzed logic gates in a biochemical sensing system to perform an enzyme-biocatalyzed reaction resembling a Boolean logic operation on biomarker inputs (6510). The enzyme-biocatalyzed logic gates can generate an output of the enzyme-biocatalyzed reaction (6520). A signal processing unit in the biochemical sensing system can be operated to generate a digital binary output based on the generated output of the enzyme-biocatalyzed reaction (6530). The digital binary output indicates presence or absence of a medical condition.

The network of enzyme-biocatalyzed logic gates can process the biomarker inputs that includes one or a combination of multiple biomarkers associated with a particular type of a medical condition (6512). The network of enzyme-biocatalyzed logic gates can perform enzyme-biocatalyzed reactions in parallel (6514) and generate outputs of the enzyme-biocatalyzed reactions in parallel (6522). The network of enzyme-biocatalyzed logic gates can process multiple biomarker inputs associated with a given medical conditions (6516). In addition, the network of enzyme-biocatalyzed logic gates can be operated to switch between different modes of operations associated with different medical conditions or injuries (6518).

Also, the signal processing unit can generate digital binary outputs in parallel in response to the parallelly generated outputs of the enzyme-biocatalyzed reactions to detect multiple medical conditions (6532). The signal processing unit can generate an injury code to include a multi-bit digital word that indicates an injury (6534).

The biochemical system and the associated components including a network of enzyme-biocatalyzed logic gates described in FIG. 65 can be implemented to be substantially similar to those described with respect to FIGS. 1-64.

While this specification contains many specifics, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments.

Only a few implementations and examples are described and other implementations, enhancements and variations can be made based on what is described and illustrated in this application.

What is claimed is:

1. A biochemical logic sensing system, comprising:
a network of enzyme-biocatalyzed logic gates adapted to receive biomarker inputs and perform an enzyme-biocatalyzed reaction resembling a Boolean logic operation using the received biomarker inputs to generate an output of the enzyme-biocatalyzed reaction; and
a signal processing unit connected to the network of enzyme-biocatalyzed logic gates, the signal processing unit to process the generated output of the enzyme-biocatalyzed reaction and generate a digital binary output having a value of zero or one,
wherein the generated digital binary output indicates an injury.

2. The biochemical logic sensing system of claim 1, further comprising:
a drug delivery device connected to the signal processing unit, the drug delivery device identifying a dosage of a drug to deliver based on the processed digital binary output.

3. The biochemical logic sensing system of claim 1, wherein the signal processing unit is adapted to generate the digital binary output having a value of 0 when the network of enzyme-biocatalyzed logic gates detects a physiologically normal concentration of the biomarker inputs; and
wherein the signal processing unit is adapted to generate the digital binary output having a value of 1 when the network of enzyme-biocatalyzed logic gates detects an abnormal concentration of the biomarker input.

4. The biochemical logic sensing system of claim 1, wherein the biomarker inputs comprise glucose, lactate, norepinephrine (NE) and oxygen.

5. The biochemical logic sensing system of claim 4, wherein the signal processing unit is adapted to generate the digital binary output having a value of 1 when the network of enzyme-biocatalyzed logic gates detects at least one of:
an abnormal increase in glucose concentration associated with hemorrhagic shock (HS);
a higher than normal physiological concentration of lactate associated with HS and/or trauma brain injury (TBI);
a high concentration of NE associated with a traumatic injury; and
lack of oxygen associated with ischemic state and/or heart attack.

6. The biochemical logic sensing system of claim 1, wherein the network of enzyme-biocatalyzed logic gates comprises at least one of an AND gate, an XOR gate, an OR gate, a NAND gate, a NOR gate and an IDENTITY gate.

7. The biochemical logic sensing system of claim 1, wherein the network of enzyme-biocatalyzed logic gates are adapted to form a cascade arrangement.

8. The biochemical logic sensing system of claim 1, wherein the network of enzyme-biocatalyzed logic gates is adapted to form parallel-operating enzyme-catalyzed pathways producing outputs of two or more enzyme-biocatalyzed reactions in parallel.

9. The biochemical logic sensing system of claim 8, wherein the signal processing unit is adapted to:
generate two or more digital binary output in parallel based on the outputs of two or more enzyme-biocatalyzed reactions performed in parallel; and
identify a type of an injury based on at least one of the generated digital binary output signals.

10. The biochemical logic sensing system of claim 1, further comprising:
a monitoring device connected to the signal processing unit, the monitoring device adapted to monitor the output of the enzyme-biocatalyzed reaction.

11. The biochemical logic sensing system of claim 1, wherein the monitoring device comprises at least one of an optical monitor and an electrochemical monitor.

12. The biochemical logic sensing system of claim 1, wherein the biomarker inputs comprise lactate, norepinephrine and glucose; and
the network of enzyme-biocatalyzed logic gates comprises at least one of an AND gate and an IDENTITY gate adapted to operate in concert using lactate oxidase, horseradish peroxidase and glucose dehydrogenase enzymes.

13. The biochemical logic sensing system of claim 1, wherein the network of enzyme-biocatalyzed logic gates is adapted to process different patterns of the biomarker inputs to generate the output of the enzyme-biocatalyzed reaction comprising norepi-quinone and reduced dipotassium salt (NADH).

14. The biochemical logic sensing system of claim 1, wherein the network of enzyme-biocatalyzed logic gates is adapted to process the biomarker inputs to provide diagnostic information on multiple conditions at a point-of-care.

15. The biochemical logic sensing system of claim 1, wherein the network of enzyme-biocatalyzed logic gates is adapted to process the biomarker inputs to provide diagnostic information on a cardiovascular event.

16. The biochemical logic sensing system of claim 1, wherein the network of enzyme-biocatalyzed logic gates is adapted to process the biomarker inputs comprising myeloperoxidase (MPO), lactate dehydrogenase (LDH) and creatine kinase (CK), and generate the output signals comprising reduced dipotassium salt (NADH) and oxidized redox mediator (Mox).

17. The biochemical logic sensing system of claim 1, wherein the signal processing unit is adapted to process the output of the enzyme-biocatalyzed reaction comprising reduced dipotassium salt (NADH) and oxidized redox mediator (Mox) to generate the digital binary output that indicates at least one of four possible symptoms comprising a healthy heart (0,0), unhealthy body tissue (1,0), a heart related illness different from Acute myocardial infarction (AMI) (0,1) and AMI (1,1).

18. The biochemical logic sensing system of claim 1, wherein the network of enzyme-biocatalyzed logic gates is adapted to analyze the biomarker inputs that are characteristic of at least one of liver injury (LI), soft tissue injury (STI) and abdominal trauma (ABT).

19. The biochemical logic sensing system of claim 1, wherein the signal processing unit is adapted to use the generated the digital binary output to produce an alert-type optical output signal in the form of YES-NO separated by a threshold value.

20. The biochemical logic sensing system of claim 1, wherein the network of enzyme-biocatalyzed logic gates comprises at least one of AND, OR, NAND, NOR, and XOR gates.

21. The biochemical logic sensing system of claim 1, wherein the network of enzyme-biocatalyzed logic gates is adapted to process the biomarker inputs comprising alanine transaminase (ALT) and lactate dehydrogenase (LDH) associated with liver injury.

22. The biochemical logic sensing system of claim 1, wherein the network of enzyme-biocatalyzed logic gates is adapted to process the biomarker inputs comprising CK and LDH associated with soft tissue injury.

23. The biochemical logic sensing system of claim 1, wherein the network of enzyme-biocatalyzed logic gates is adapted to process the biomarker inputs comprising enzyme LDH and its substrate Lac at elevated concentrations associated with abdominal trauma (ABT).

24. The biochemical logic sensing system of claim 1, wherein the network of enzyme-biocatalyzed logic gates is adapted to process simultaneously different combinations of five biomarker inputs characteristic of traumatic brain injury (TBI) and soft tissue injury (STI).

25. The biochemical logic sensing system of claim 24, wherein three of the biomarker inputs comprise creatine kinase (CK), lactate dehydrogenase (LDH) and lactate (Lac) associated with physiological conditions characteristic of STI.

26. The biochemical logic sensing system of claim 24, wherein two of the biomarker inputs comprise enolase (EN) and glutamate (Glu) associated with the TBI diagnosis.

27. The biochemical logic sensing system of claim 24, wherein the network of enzyme-biocatalyzed logic gates is adapted to switch between an STI detection mode and a TBI detection mode.

28. The biochemical logic sensing system of claim 24, wherein the network of enzyme-biocatalyzed logic gates is adapted to process simultaneous presence of elevated levels of CK and LDH to trigger a positive diagnosis of STI.

29. The biochemical logic sensing system of claim 1, further comprising a disposable electrode to obtain a blood sample.

30. The biochemical logic sensing system of claim 1, wherein the disposable electrode comprises a flexible carbon screen-printed electrode (SPE).

31. The biochemical logic sensing system of claim 1, wherein the network of enzyme-biocatalyzed logic gates is adapted to assess the biomarker inputs comprising two or more selected from creatine kinase, lactate dehydrogenase, norepinephrine, glutamate, alanine transaminase, lactate, glucose, glutathione disulfide, and glutathione reductase to assess associated one of soft-tissue injury, traumatic brain injury, liver injury, abdominal trauma, hemorrhagic shock, and oxidative stress.

32. The biochemical logic sensing system of claim 1, wherein the signal processing unit is adapted to generate a digitally-encoded multi-bit binary word that represents an injury code.

33. The biochemical logic sensing system of claim 1, wherein the network of enzyme-biocatalyzed logic gates are arranged in parallel and multiplexed.

34. The biochemical logic sensing system of claim 1, wherein the network of enzyme-biocatalyzed logic gates is adapted to form different arrangements with each enzyme-biocatalyzed logic gate receiving two of the biomarker inputs.

35. The biochemical logic sensing system of claim 1, wherein the network of enzyme-biocatalyzed logic gates are adapted to process the biomarker inputs that comprise Glutathione disulfide (GSSG) and glucocorticoid receptor (GR) associated with oxidative stress.

36. The biochemical logic sensing system of claim 1, wherein the network of enzyme-biocatalyzed logic gates is adapted to process the biomarker inputs comprising alanine transaminase (ALT) and lactate dehydrogenase (LDH) to generate NAD output signal; and
wherein the biochemical logic sensing system further comprises a switchable polymer-modified electrode connected to the signal processing unit, the switchable polymer-modified electrode adapted convert the generated NAD output signal to a change in pH.

37. The biochemical logic sensing system of claim 1, wherein the network of enzyme-biocatalyzed logic gates comprises:
a first AND logic gate activated by lactate dehydrogenase (LDH) and lactate—jointly representing biomarker inputs for abdominal trauma (ABT); and
a second AND logic gate was activated by glutathione reductase (GR) and glutathione disulfide (GSSG) representative of oxidative stress when their concentrations are elevated.

38. The biochemical logic sensing system of claim 1, wherein the network of enzyme-biocatalyzed logic gates is adapted to process the biomarker inputs comprising α-amylase (Amy) and lactate dehydrogenase (LDH) to analyze radiation-caused tissue damage.

39. The biochemical logic sensing system of claim 1, wherein the network of enzyme-biocatalyzed logic gates is adapted to process the biomarker inputs to detect the presence of at least one of explosive compounds and nerve agents.

40. The biochemical logic sensing system of claim 39, wherein the network of enzyme-biocatalyzed logic gates is adapted to process the biomarker inputs that comprises 2,4,6-trinitrotoluene (TNT) and paraoxon (PAX) to detection 2,4-dinitrotoluene (DNT) explosive and methyl-parathion (MPT) nerve agent.

41. The biochemical logic sensing system of claim 1, wherein the network of enzyme-biocatalyzed logic gates is adapted to utilize enzymes comprising zanitroreductase (NRd), horseradish peroxidase (HRP), acetylcholinesterase (AChE), and choline oxidase (ChOx).

42. The biochemical logic sensing system of claim 1, wherein the network of enzyme-biocatalyzed logic gates is adapted to simultaneously process biochemically coupled diabetes biomarker inputs comprising glucose and hemoglobin A1c (HbA1c) to generate the output of the enzyme-biocatalyzed reaction; and
the signal processing unit is adapted to process the generated output of the enzyme-biocatalyzed reaction to generate the digital binary output indicative of diabetes.

43. The biochemical logic sensing system of claim 1, wherein the network of enzyme-biocatalyzed logic gates is disposed on a single use test strip.

44. The biochemical logic sensing system of claim 43, wherein the single use test strip comprises:
a reporter anti-HbA1c polyclonal antibody labeled with a horseradish peroxidase (HRP), which is deposited in a sampling zone patch; and
a capture anti-HbA1c analyte-specific polyclonal antibody immobilized on a surface of a detection zone together with glucose oxidase (GOx) and dried redox dye.

45. The biochemical logic sensing system of claim 44, wherein the single use test strip is adapted to:
receive a liquid sample comprising glucose onto the sample zone to solubilize the labeled reporter antibody, which binds to the HbA1c to for an analyte-antibody complex;
when the analyte-antibody complex flows with the liquid sample laterally along the surface of the test strip over the detection zone, the analyte-antibody complex binds to the capture antibody to accumulate at the capture zone of the test strip; and
the immobilized GOx converts glucose present in the sample to hydrogen peroxidse, which serves as a substrate for the HRP-labeled reporter antibody present on the HbA1c molecule.

46. A method of detecting a medical condition using a biochemical sensing system comprising:
operating a network of enzyme-biocatalyzed logic gates in a biochemical sensing system to perform an enzyme-biocatalyzed reaction resembling a Boolean logic operation on biomarker inputs to generate an output of the enzyme-biocatalyzed reaction;
operating a signal processing unit in the biochemical sensing system to generate a digital binary output based on the generated output of the enzyme-biocatalyzed reaction, wherein the digital binary output indicates presence or absence of a medical condition.

47. The method of claim 46, comprising:
operating a drug deliver device to determine a dosage of a drug to deliver based on the digital binary output from the signal processing unit.

48. The method of claim 1, comprising:
operating a monitoring device to analyze the output of the enzyme-biocatalyzed reaction.

49. The method of claim 1, comprising:
operating the network of enzyme-biocatalyzed logic gates to process the biomarker inputs that includes one or a combination of multiple biomarkers associated with a particular type of a medical condition.

50. The method of claim 1, comprising:
operating the network of enzyme-biocatalyzed logic gates to perform enzyme-biocatalyzed reactions in parallel and generate outputs of the enzyme-biocatalyzed reactions in parallel; and
operating the signal processing unit to generate digital binary outputs in parallel in response to the parallelly generated outputs of the enzyme-biocatalyzed reactions to detect multiple medical conditions.

51. The method of claim 1, comprising:
operating the signal processing unit to generate an injury code comprising a multi-bit digital word.

52. The method of claim 1, comprising:
operating the network of enzyme-biocatalyzed logic gates to process multiple biomarker inputs associated with a given medical condition.

53. The method of claim 1, comprising:
operating the network of enzyme-biocatalyzed logic gates to switch between multiple modes of operation.
